SEQUENCE LISTING (421 sequences)

SEQ ID NO: 1 - protein, 11 aa, Artificial sequence
  Synthetic sequence
ACTGSTQHQCG

SEQ ID NO: 2 - protein, 9 aa, Artificial sequence
  Synthetic sequence
ACSHNHTCG

SEQ ID NO: 3 - protein, 10 aa, Artificial sequence
  Synthetic sequence
ACTHTGRNCG

SEQ ID NO: 4 - protein, 11 aa, Artificial sequence
  Synthetic sequence
ACSATLQHSCG

SEQ ID NO: 5 - protein, 12 aa, Artificial sequence
  Synthetic sequence
ACNAHQARSTCG

SEQ ID NO: 6 - protein, 11 aa, Artificial sequence
  Synthetic sequence
ACSLTVNWNCG

SEQ ID NO: 7 - protein, 11 aa, Artificial sequence
  Synthetic sequence
ACLSVNHNACG

SEQ ID NO: 8 - protein, 11 aa, Artificial sequence
  Synthetic sequence
ACSASTNHNCG

SEQ ID NO: 9 - protein, 11 aa, Artificial sequence
  Synthetic sequence
ACSASQVHNCG

SEQ ID NO: 10 - protein, 11 aa, Artificial sequence
  Synthetic sequence
ACNGTGSHQCG

SEQ ID NO: 11 - protein, 11 aa, Artificial sequence
  Synthetic sequence
ACSVTTQHQCG

SEQ ID NO: 12 - protein, 11 aa, Artificial sequence
  Synthetic sequence
ACVSVTNHQCG

SEQ ID NO: 13 - protein, 11 aa, Artificial sequence
  Synthetic sequence
ACTSTGRNACG

SEQ ID NO: 14 - protein, 11 aa, Artificial sequence
  Synthetic sequence
ACKTGSHNQCG

SEQ ID NO: 15 - protein, 11 aa, Artificial sequence
  Synthetic sequence
ACGSGSGSGCG

SEQ ID NO: 16 - protein, 103 aa, Artificial sequence
  Synthetic sequence
  MISC_FEATURE (1)..(1): A disulfide bond is present  between the two C residues in this sequence
  MISC_FEATURE (2)..(2): X at this position is any amino acid excluding cysteine
  MISC_FEATURE (3)..(102): X at these positions is absent or present such that anywhere from 0-100 Xs are present, and when X is present X is any amino acid excluding cysteine.
  MISC_FEATURE (103)..(103): A disulfide bond is present  between the two C residues in this sequence
CXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXC

SEQ ID NO: 17 - protein, 105 aa, Artificial sequence
  Synthetic sequence
  MISC_FEATURE (2)..(2): A disulfide bond is present  between the two C residues in this sequence
  MISC_FEATURE (3)..(3): X at this position is any amino acid excluding cysteine
  MISC_FEATURE (4)..(103): X at these positions is absent or present such that anywhere from 0-100 Xs are present, and when X is present X is any amino acid excluding cysteine.
  MISC_FEATURE (104)..(104): A disulfide bond is present  between the two C residues in this sequence
ACXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCG

SEQ ID NO: 18 - protein, 7 aa, Artificial sequence
  Synthetic sequence
  MISC_FEATURE (1)..(1): A disulfide bond is present  between the two C residues in this sequence
  MISC_FEATURE (2)..(2): X at this position is Serine, Threonine, Asparagine, Glutamine, or Glycine
  MISC_FEATURE (3)..(3): X at this position is Histidine, Asparagine, or Glutamine
  MISC_FEATURE (4)..(4): X at this position is Histidine, Arginine, Asparagine, or Glutamine
  MISC_FEATURE (5)..(5): X at this position is Histidine, Asparagine, Glutamine, Serine, or Threonine
  MISC_FEATURE (6)..(6): X at this position is Serine, Threonine, Glycine, or Alanine
  MISC_FEATURE (7)..(7): A disulfide bond is present  between the two C residues in this sequence
CXXXXXC

SEQ ID NO: 19 - protein, 8 aa, Artificial sequence
  Synthetic sequence
  MISC_FEATURE (1)..(1): A disulfide bond is present  between the two C residues in this sequence
  MISC_FEATURE (2)..(2): X at this position is Serine, Threonine, Asparagine, Glutamine, or Glycine;
  MISC_FEATURE (3)..(3): X at this position is Serine, Threonine, Asparagine, or Glutamine
  MISC_FEATURE (4)..(4): X at this position is Histidine, Arginine, Lysine, Asparagine, Glutamine, Glycine, or Alanine
  MISC_FEATURE (5)..(5): X at this position is Serine, Threonine, Asparagine, Glutamine, Glycine, or Arginine
  MISC_FEATURE (6)..(6): X at this position is Histidine, Arginine, Lysine, Asparagine, Glutamine, Serine, or Threonine
  MISC_FEATURE (7)..(7): X at this position is Asparagine, Glutamine, Serine, Threonine, Arginine, Glycine, or Alanine
  MISC_FEATURE (8)..(8): A disulfide bond is present  between the two C residues in this sequence
CXXXXXXC

SEQ ID NO: 20 - protein, 9 aa, Artificial sequence
  Synthetic sequence
  MISC_FEATURE (1)..(1): A disulfide bond is present  between the two C residues in this sequence
  MISC_FEATURE (2)..(2): X at this position is Serine, Threonine, Glycine, Alanine, or Valine
  MISC_FEATURE (3)..(3): X at this position is Glycine, Alanine, Valine, Leucine, Serine, or Threonine
  MISC_FEATURE (4)..(4): X at this position is Glycine, Alanine, Serine, or Threonine
  MISC_FEATURE (5)..(5): X at this position is Asparagine, Glutamine, Arginine, or Lysine
  MISC_FEATURE (6)..(6): X at this position is Histidine, Asparagine, Glutamine, Tryptophan, Serine, or Threonine
  MISC_FEATURE (7)..(7): X at this position is Serine, Threonine, Histidine, Asparagine, Glutamine, Glycine, or Alanine
  MISC_FEATURE (8)..(8): X at this position is Serine, Threonine, Histidine, Asparagine, Glutamine, Glycine, or Alanine
  MISC_FEATURE (9)..(9): A disulfide bond is present  between the two C residues in this sequence
CXXXXXXXC

SEQ ID NO: 21 - protein, 10 aa, Artificial sequence
  Synthetic sequence
  MISC_FEATURE (1)..(1): A disulfide bond is present  between the two C residues in this sequence
  MISC_FEATURE (2)..(2): X at this position is Serine, Threonine, Asparagine, Glutamine, Glycine, or Alanine
  MISC_FEATURE (3)..(3): X at this position is Alanine, Serine, Threonine, or Arginine
  MISC_FEATURE (4)..(4): X at this position is Histidine, Asparagine, Glutamine, Lysine, or Arginine
  MISC_FEATURE (5)..(5): X at this position is Asparagine, Arginine, Histidine, or Tryptophan
  MISC_FEATURE (6)..(6): X at this position is Glycine, Alanine, Arginine, Glutamine, Lysine, or Arginine
  MISC_FEATURE (7)..(7): X at this position is Histidine, Tryptophan, Glycine, or Alanine
  MISC_FEATURE (8)..(8): X at this position is Serine, Threonine, Asparagine, or Glutamine
  MISC_FEATURE (9)..(9): X at this position is Serine, Threonine, Glycine, or Alanine
  MISC_FEATURE (10)..(10): A disulfide bond is present  between the two C residues in this sequence
CXXXXXXXXC

SEQ ID NO: 22 - protein, 13 aa, Artificial sequence
  Synthetic sequence
  MISC_FEATURE (3)..(3): X at this position is any amino acid excluding cysteine
  MISC_FEATURE (4)..(11): 5, 6, 7, or 8 Xs are present at this position, and X is any amino acid excluding cysteine.
ACXXXXXXXXXCG

SEQ ID NO: 23 - protein, 11 aa, Artificial sequence
  Synthetic sequence
ACSSSPSKHCG

SEQ ID NO: 24 - protein, 7 aa, Artificial sequence
  Synthetic sequence
RRRRRRR

SEQ ID NO: 25 - protein, 12 aa, Artificial sequence
  Synthetic sequence
HIITDPNMAEYL

SEQ ID NO: 26 - protein, 11 aa, Artificial sequence
  Synthetic sequence
ACTIQHRAECG

SEQ ID NO: 27 - protein, 11 aa, Artificial sequence
  Synthetic sequence
ACTIQHGRSCG

SEQ ID NO: 28 - protein, 11 aa, Artificial sequence
  Synthetic sequence
ACVSAGQNHCG

SEQ ID NO: 29 - protein, 11 aa, Artificial sequence
  Synthetic sequence
ACVSIGNHNCG

SEQ ID NO: 30 - protein, 11 aa, Artificial sequence
  Synthetic sequence
ACVSANHQICG

SEQ ID NO: 31 - protein, 11 aa, Artificial sequence
  Synthetic sequence
ACVSATGNHCG

SEQ ID NO: 32 - protein, 11 aa, Artificial sequence
  Synthetic sequence
ACAEGINVHCG

SEQ ID NO: 33 - protein, 11 aa, Artificial sequence
  Synthetic sequence
ACVSEHINGCG

SEQ ID NO: 34 - protein, 11 aa, Artificial sequence
  Synthetic sequence
ACTIQHRAFCG

SEQ ID NO: 35 - protein, 11 aa, Artificial sequence
  Synthetic sequence
ACRVAHFITCG

SEQ ID NO: 36 - protein, 11 aa, Artificial sequence
  Synthetic sequence
ACVSEQHNICG

SEQ ID NO: 37 - protein, 11 aa, Artificial sequence
  Synthetic sequence
ACVSANHQTCG

SEQ ID NO: 38 - protein, 11 aa, Artificial sequence
  Synthetic sequence
ACTSVINEHCG

SEQ ID NO: 39 - protein, 11 aa, Artificial sequence
  Synthetic sequence
ACAEHRSQTCG

SEQ ID NO: 40 - protein, 11 aa, Artificial sequence
  Synthetic sequence
ACRVTNHQSCG

SEQ ID NO: 41 - protein, 11 aa, Artificial sequence
  Synthetic sequence
ACTIHNRQSCG

SEQ ID NO: 42 - protein, 11 aa, Artificial sequence
  Synthetic sequence
ACVSENHQGCG

SEQ ID NO: 43 - protein, 11 aa, Artificial sequence
  Synthetic sequence
ACVSAGNHQCG

SEQ ID NO: 44 - protein, 11 aa, Artificial sequence
  Synthetic sequence
ACRVAIHGNCG

SEQ ID NO: 45 - protein, 11 aa, Artificial sequence
  Synthetic sequence
ACVSATQHNCG

SEQ ID NO: 46 - protein, 11 aa, Artificial sequence
  Synthetic sequence
ACVSATFNHCG

SEQ ID NO: 47 - protein, 11 aa, Artificial sequence
  Synthetic sequence
ACTHNRQSFCG

SEQ ID NO: 48 - protein, 11 aa, Artificial sequence
  Synthetic sequence
ACIEVNHNRCG

SEQ ID NO: 49 - protein, 11 aa, Artificial sequence
  Synthetic sequence
ACVSEFNTHCG

SEQ ID NO: 50 - protein, 11 aa, Artificial sequence
  Synthetic sequence
ACHISGVFNCG

SEQ ID NO: 51 - protein, 11 aa, Artificial sequence
  Synthetic sequence
ACIEVNHNSCG

SEQ ID NO: 52 - protein, 11 aa, Artificial sequence
  Synthetic sequence
ACHISGEARCG

SEQ ID NO: 53 - protein, 11 aa, Artificial sequence
  Synthetic sequence
ACIVNHFRQCG

SEQ ID NO: 54 - protein, 11 aa, Artificial sequence
  Synthetic sequence
ACHSAGIFVCG

SEQ ID NO: 55 - protein, 11 aa, Artificial sequence
  Synthetic sequence
ACIEVNHQSCG

SEQ ID NO: 56 - protein, 11 aa, Artificial sequence
  Synthetic sequence
ACVIHFTRNCG

SEQ ID NO: 57 - protein, 11 aa, Artificial sequence
  Synthetic sequence
ACGVQHSRNCG

SEQ ID NO: 58 - protein, 11 aa, Artificial sequence
  Synthetic sequence
ACVSNQIERCG

SEQ ID NO: 59 - protein, 11 aa, Artificial sequence
  Synthetic sequence
ACVSTGNHNCG

SEQ ID NO: 60 - protein, 11 aa, Artificial sequence
  Synthetic sequence
ACTHENRQSCG

SEQ ID NO: 61 - protein, 11 aa, Artificial sequence
  Synthetic sequence
ACVSAFTHGCG

SEQ ID NO: 62 - protein, 11 aa, Artificial sequence
  Synthetic sequence
ACIEANTFHCG

SEQ ID NO: 63 - protein, 11 aa, Artificial sequence
  Synthetic sequence
ACAEQGHTRCG

SEQ ID NO: 64 - protein, 11 aa, Artificial sequence
  Synthetic sequence
ACTQVNHRSCG

SEQ ID NO: 65 - protein, 11 aa, Artificial sequence
  Synthetic sequence
ACAVRENQTCG

SEQ ID NO: 66 - protein, 11 aa, Artificial sequence
  Synthetic sequence
ACTIANRQSCG

SEQ ID NO: 67 - protein, 11 aa, Artificial sequence
  Synthetic sequence
ACTIVNHRSCG

SEQ ID NO: 68 - protein, 11 aa, Artificial sequence
  Synthetic sequence
ACAEIVGHRCG

SEQ ID NO: 69 - protein, 11 aa, Artificial sequence
  Synthetic sequence
ACRVNHTSICG

SEQ ID NO: 70 - protein, 11 aa, Artificial sequence
  Synthetic sequence
ACAEIHTGRCG

SEQ ID NO: 71 - protein, 11 aa, Artificial sequence
  Synthetic sequence
ACRVNHTSACG

SEQ ID NO: 72 - protein, 11 aa, Artificial sequence
  Synthetic sequence
ACRVNHASQCG

SEQ ID NO: 73 - protein, 11 aa, Artificial sequence
  Synthetic sequence
ACVSNAQERCG

SEQ ID NO: 74 - protein, 11 aa, Artificial sequence
  Synthetic sequence
ACQVTEIANCG

SEQ ID NO: 75 - protein, 11 aa, Artificial sequence
  Synthetic sequence
ACHTNAVSQCG

SEQ ID NO: 76 - protein, 11 aa, Artificial sequence
  Synthetic sequence
ACQVNHFISCG

SEQ ID NO: 77 - protein, 11 aa, Artificial sequence
  Synthetic sequence
ACQVNFASTCG

SEQ ID NO: 78 - protein, 11 aa, Artificial sequence
  Synthetic sequence
ACHVQGENSCG

SEQ ID NO: 79 - protein, 11 aa, Artificial sequence
  Synthetic sequence
ACQRHVASTCG

SEQ ID NO: 80 - protein, 11 aa, Artificial sequence
  Synthetic sequence
ACTSRNQHVCG

SEQ ID NO: 81 - protein, 11 aa, Artificial sequence
  Synthetic sequence
ACRVSFHTQCG

SEQ ID NO: 82 - protein, 11 aa, Artificial sequence
  Synthetic sequence
ACQRITSHACG

SEQ ID NO: 83 - protein, 11 aa, Artificial sequence
  Synthetic sequence
ACRAHFGESCG

SEQ ID NO: 84 - protein, 11 aa, Artificial sequence
  Synthetic sequence
ACAEIGRNSCG

SEQ ID NO: 85 - protein, 11 aa, Artificial sequence
  Synthetic sequence
ACTINHRVSCG

SEQ ID NO: 86 - protein, 11 aa, Artificial sequence
  Synthetic sequence
ACQVFATSHCG

SEQ ID NO: 87 - protein, 11 aa, Artificial sequence
  Synthetic sequence
ACSRVNTGQCG

SEQ ID NO: 88 - protein, 11 aa, Artificial sequence
  Synthetic sequence
ACTINHRSVCG

SEQ ID NO: 89 - protein, 11 aa, Artificial sequence
  Synthetic sequence
ACTIHSVQNCG

SEQ ID NO: 90 - protein, 11 aa, Artificial sequence
  Synthetic sequence
ACQVTAGRSCG

SEQ ID NO: 91 - protein, 11 aa, Artificial sequence
  Synthetic sequence
ACTNAIRFSCG

SEQ ID NO: 92 - protein, 11 aa, Artificial sequence
  Synthetic sequence
ACQVAGIHNCG

SEQ ID NO: 93 - protein, 11 aa, Artificial sequence
  Synthetic sequence
ACTIEGFANCG

SEQ ID NO: 94 - protein, 11 aa, Artificial sequence
  Synthetic sequence
ACTNFEGSRCG

SEQ ID NO: 95 - protein, 11 aa, Artificial sequence
  Synthetic sequence
ACQVNRASHCG

SEQ ID NO: 96 - protein, 11 aa, Artificial sequence
  Synthetic sequence
ACIVQANERCG

SEQ ID NO: 97 - protein, 11 aa, Artificial sequence
  Synthetic sequence
ACVFSNQITCG

SEQ ID NO: 98 - protein, 11 aa, Artificial sequence
  Synthetic sequence
ACVFSITGQCG

SEQ ID NO: 99 - protein, 11 aa, Artificial sequence
  Synthetic sequence
ACVSHTNRFCG

SEQ ID NO: 100 - protein, 11 aa, Artificial sequence
  Synthetic sequence
ACVSGFETACG

SEQ ID NO: 101 - protein, 11 aa, Artificial sequence
  Synthetic sequence
ACRVAQTGICG

SEQ ID NO: 102 - protein, 11 aa, Artificial sequence
  Synthetic sequence
ACVIRQSNTCG

SEQ ID NO: 103 - protein, 10 aa, Artificial sequence
  Synthetic sequence
ACVSETRNIC

SEQ ID NO: 104 - protein, 10 aa, Artificial sequence
  Synthetic sequence
ACVNARISFC

SEQ ID NO: 105 - protein, 11 aa, Artificial sequence
  Synthetic sequence
ACAEIFGQNCG

SEQ ID NO: 106 - protein, 11 aa, Artificial sequence
  Synthetic sequence
ACAERSGIVCG

SEQ ID NO: 107 - protein, 11 aa, Artificial sequence
  Synthetic sequence
ACAEHNISQCG

SEQ ID NO: 108 - protein, 11 aa, Artificial sequence
  Synthetic sequence
ACNGTGSHQCG

SEQ ID NO: 109 - protein, 11 aa, Artificial sequence
  Synthetic sequence
ACVSFINTQCG

SEQ ID NO: 110 - protein, 11 aa, Artificial sequence
  Synthetic sequence
ACSRQHNEFCG

SEQ ID NO: 111 - protein, 11 aa, Artificial sequence
  Synthetic sequence
ACQNFIERACG

SEQ ID NO: 112 - protein, 11 aa, Artificial sequence
  Synthetic sequence
ACVSFGIENCG

SEQ ID NO: 113 - protein, 11 aa, Artificial sequence
  Synthetic sequence
ACVNTERFGCG

SEQ ID NO: 114 - protein, 11 aa, Artificial sequence
  Synthetic sequence
ACNSTAVQGCG

SEQ ID NO: 115 - protein, 11 aa, Artificial sequence
  Synthetic sequence
ACNSITERVCG

SEQ ID NO: 116 - protein, 11 aa, Artificial sequence
  Synthetic sequence
ACVSNEFGTCG

SEQ ID NO: 117 - protein, 11 aa, Artificial sequence
  Synthetic sequence
ACAEIQGNRCG

SEQ ID NO: 118 - protein, 11 aa, Artificial sequence
  Synthetic sequence
ACHSALTKHCG

SEQ ID NO: 119 - protein, 11 aa, Artificial sequence
  Synthetic sequence
ACMGPSSMLCG

SEQ ID NO: 120 - protein, 11 aa, Artificial sequence
  Synthetic sequence
ACTDPNQLQCG

SEQ ID NO: 121 - protein, 11 aa, Artificial sequence
  Synthetic sequence
ACSTHFIDTCG

SEQ ID NO: 122 - protein, 5 aa, Artificial sequence
  Synthetic sequence
AAAAA

SEQ ID NO: 123 - protein, 5 aa, Artificial sequence
  Synthetic sequence
AAAAA

SEQ ID NO: 124 - protein, 5 aa, Artificial sequence
  Synthetic sequence
AAAAA

SEQ ID NO: 125 - protein, 5 aa, Artificial sequence
  Synthetic sequence
AAAAA

SEQ ID NO: 126 - protein, 5 aa, Artificial sequence
  Synthetic sequence
AAAAA

SEQ ID NO: 127 - protein, 5 aa, Artificial sequence
  Synthetic sequence
AAAAA

SEQ ID NO: 128 - protein, 5 aa, Artificial sequence
  Synthetic sequence
AAAAA

SEQ ID NO: 129 - protein, 5 aa, Artificial sequence
  Synthetic sequence
AAAAA

SEQ ID NO: 130 - protein, 5 aa, Artificial sequence
  Synthetic sequence
AAAAA

SEQ ID NO: 131 - protein, 5 aa, Artificial sequence
  Synthetic sequence
AAAAA

SEQ ID NO: 132 - protein, 5 aa, Artificial sequence
  Synthetic sequence
AAAAA

SEQ ID NO: 133 - protein, 5 aa, Artificial sequence
  Synthetic sequence
AAAAA

SEQ ID NO: 134 - protein, 5 aa, Artificial sequence
  Synthetic sequence
AAAAA

SEQ ID NO: 135 - protein, 5 aa, Artificial sequence
  Synthetic sequence
AAAAA

SEQ ID NO: 136 - protein, 5 aa, Artificial sequence
  Synthetic sequence
AAAAA

SEQ ID NO: 137 - protein, 5 aa, Artificial sequence
  Synthetic sequence
AAAAA

SEQ ID NO: 138 - protein, 5 aa, Artificial sequence
  Synthetic sequence
AAAAA

SEQ ID NO: 139 - protein, 5 aa, Artificial sequence
  Synthetic sequence
AAAAA

SEQ ID NO: 140 - protein, 5 aa, Artificial sequence
  Synthetic sequence
AAAAA

SEQ ID NO: 141 - protein, 5 aa, Artificial sequence
  Synthetic sequence
AAAAA

SEQ ID NO: 142 - protein, 5 aa, Artificial sequence
  Synthetic sequence
AAAAA

SEQ ID NO: 143 - protein, 5 aa, Artificial sequence
  Synthetic sequence
AAAAA

SEQ ID NO: 144 - protein, 5 aa, Artificial sequence
  Synthetic sequence
AAAAA

SEQ ID NO: 145 - protein, 5 aa, Artificial sequence
  Synthetic sequence
AAAAA

SEQ ID NO: 146 - protein, 5 aa, Artificial sequence
  Synthetic sequence
AAAAA

SEQ ID NO: 147 - protein, 5 aa, Artificial sequence
  Synthetic sequence
AAAAA

SEQ ID NO: 148 - protein, 5 aa, Artificial sequence
  Synthetic sequence
AAAAA

SEQ ID NO: 149 - protein, 5 aa, Artificial sequence
  Synthetic sequence
AAAAA

SEQ ID NO: 150 - protein, 5 aa, Artificial sequence
  Synthetic sequence
AAAAA

SEQ ID NO: 151 - protein, 9 aa, Artificial sequence
  Synthetic sequence
CTGSTQHQC

SEQ ID NO: 152 - protein, 7 aa, Artificial sequence
  Synthetic sequence
CSHNHTC

SEQ ID NO: 153 - protein, 8 aa, Artificial sequence
  Synthetic sequence
CTHTGRNC

SEQ ID NO: 154 - protein, 9 aa, Artificial sequence
  Synthetic sequence
CSATLQHSC

SEQ ID NO: 155 - protein, 10 aa, Artificial sequence
  Synthetic sequence
CNAHQARSTC

SEQ ID NO: 156 - protein, 9 aa, Artificial sequence
  Synthetic sequence
CSLTVNWNC

SEQ ID NO: 157 - protein, 9 aa, Artificial sequence
  Synthetic sequence
CLSVNHNAC

SEQ ID NO: 158 - protein, 9 aa, Artificial sequence
  Synthetic sequence
CSASTNHNC

SEQ ID NO: 159 - protein, 9 aa, Artificial sequence
  Synthetic sequence
CSASQVHNC

SEQ ID NO: 160 - protein, 9 aa, Artificial sequence
  Synthetic sequence
CNGTGSHQC

SEQ ID NO: 161 - protein, 9 aa, Artificial sequence
  Synthetic sequence
CSVTTQHQC

SEQ ID NO: 162 - protein, 9 aa, Artificial sequence
  Synthetic sequence
CVSVTNHQC

SEQ ID NO: 163 - protein, 9 aa, Artificial sequence
  Synthetic sequence
CTSTGRNAC

SEQ ID NO: 164 - protein, 9 aa, Artificial sequence
  Synthetic sequence
CKTGSHNQC

SEQ ID NO: 165 - protein, 5 aa, Artificial sequence
  Synthetic sequence
AAAAA

SEQ ID NO: 166 - protein, 5 aa, Artificial sequence
  Synthetic sequence
AAAAA

SEQ ID NO: 167 - protein, 5 aa, Artificial sequence
  Synthetic sequence
AAAAA

SEQ ID NO: 168 - protein, 5 aa, Artificial sequence
  Synthetic sequence
AAAAA

SEQ ID NO: 169 - protein, 5 aa, Artificial sequence
  Synthetic sequence
AAAAA

SEQ ID NO: 170 - protein, 5 aa, Artificial sequence
  Synthetic sequence
AAAAA

SEQ ID NO: 171 - protein, 5 aa, Artificial sequence
  Synthetic sequence
AAAAA

SEQ ID NO: 172 - protein, 5 aa, Artificial sequence
  Synthetic sequence
AAAAA

SEQ ID NO: 173 - protein, 9 aa, Artificial sequence
  Synthetic sequence
CSSSPSKHC

SEQ ID NO: 174 - protein, 5 aa, Artificial sequence
  Synthetic sequence
AAAAA

SEQ ID NO: 175 - protein, 5 aa, Artificial sequence
  Synthetic sequence
AAAAA

SEQ ID NO: 176 - protein, 9 aa, Artificial sequence
  Synthetic sequence
CTIQHRAEC

SEQ ID NO: 177 - protein, 9 aa, Artificial sequence
  Synthetic sequence
CTIQHGRSC

SEQ ID NO: 178 - protein, 9 aa, Artificial sequence
  Synthetic sequence
CVSAGQNHC

SEQ ID NO: 179 - protein, 9 aa, Artificial sequence
  Synthetic sequence
CVSIGNHNC

SEQ ID NO: 180 - protein, 9 aa, Artificial sequence
  Synthetic sequence
CVSANHQIC

SEQ ID NO: 181 - protein, 9 aa, Artificial sequence
  Synthetic sequence
CVSATGNHC

SEQ ID NO: 182 - protein, 9 aa, Artificial sequence
  Synthetic sequence
CAEGINVHC

SEQ ID NO: 183 - protein, 9 aa, Artificial sequence
  Synthetic sequence
CVSEHINGC

SEQ ID NO: 184 - protein, 9 aa, Artificial sequence
  Synthetic sequence
CTIQHRAFC

SEQ ID NO: 185 - protein, 9 aa, Artificial sequence
  Synthetic sequence
CRVAHFITC

SEQ ID NO: 186 - protein, 9 aa, Artificial sequence
  Synthetic sequence
CVSEQHNIC

SEQ ID NO: 187 - protein, 9 aa, Artificial sequence
  Synthetic sequence
CVSANHQTC

SEQ ID NO: 188 - protein, 9 aa, Artificial sequence
  Synthetic sequence
CTSVINEHC

SEQ ID NO: 189 - protein, 9 aa, Artificial sequence
  Synthetic sequence
CAEHRSQTC

SEQ ID NO: 190 - protein, 9 aa, Artificial sequence
  Synthetic sequence
CRVTNHQSC

SEQ ID NO: 191 - protein, 9 aa, Artificial sequence
  Synthetic sequence
CTIHNRQSC

SEQ ID NO: 192 - protein, 9 aa, Artificial sequence
  Synthetic sequence
CVSENHQGC

SEQ ID NO: 193 - protein, 9 aa, Artificial sequence
  Synthetic sequence
CVSAGNHQC

SEQ ID NO: 194 - protein, 9 aa, Artificial sequence
  Synthetic sequence
CRVAIHGNC

SEQ ID NO: 195 - protein, 9 aa, Artificial sequence
  Synthetic sequence
CVSATQHNC

SEQ ID NO: 196 - protein, 9 aa, Artificial sequence
  Synthetic sequence
CVSATFNHC

SEQ ID NO: 197 - protein, 9 aa, Artificial sequence
  Synthetic sequence
CTHNRQSFC

SEQ ID NO: 198 - protein, 9 aa, Artificial sequence
  Synthetic sequence
CIEVNHNRC

SEQ ID NO: 199 - protein, 9 aa, Artificial sequence
  Synthetic sequence
CVSEFNTHC

SEQ ID NO: 200 - protein, 9 aa, Artificial sequence
  Synthetic sequence
CHISGVFNC

SEQ ID NO: 201 - protein, 9 aa, Artificial sequence
  Synthetic sequence
CIEVNHNSC

SEQ ID NO: 202 - protein, 9 aa, Artificial sequence
  Synthetic sequence
CHISGEARC

SEQ ID NO: 203 - protein, 9 aa, Artificial sequence
  Synthetic sequence
CIVNHFRQC

SEQ ID NO: 204 - protein, 9 aa, Artificial sequence
  Synthetic sequence
CHSAGIFVC

SEQ ID NO: 205 - protein, 9 aa, Artificial sequence
  Synthetic sequence
CIEVNHQSC

SEQ ID NO: 206 - protein, 9 aa, Artificial sequence
  Synthetic sequence
CVIHFTRNC

SEQ ID NO: 207 - protein, 9 aa, Artificial sequence
  Synthetic sequence
CGVQHSRNC

SEQ ID NO: 208 - protein, 9 aa, Artificial sequence
  Synthetic sequence
CVSNQIERC

SEQ ID NO: 209 - protein, 9 aa, Artificial sequence
  Synthetic sequence
CVSTGNHNC

SEQ ID NO: 210 - protein, 9 aa, Artificial sequence
  Synthetic sequence
CTHENRQSC

SEQ ID NO: 211 - protein, 9 aa, Artificial sequence
  Synthetic sequence
CVSAFTHGC

SEQ ID NO: 212 - protein, 9 aa, Artificial sequence
  Synthetic sequence
CIEANTFHC

SEQ ID NO: 213 - protein, 9 aa, Artificial sequence
  Synthetic sequence
CAEQGHTRC

SEQ ID NO: 214 - protein, 9 aa, Artificial sequence
  Synthetic sequence
CTQVNHRSC

SEQ ID NO: 215 - protein, 9 aa, Artificial sequence
  Synthetic sequence
CAVRENQTC

SEQ ID NO: 216 - protein, 9 aa, Artificial sequence
  Synthetic sequence
CTIANRQSC

SEQ ID NO: 217 - protein, 9 aa, Artificial sequence
  Synthetic sequence
CTIVNHRSC

SEQ ID NO: 218 - protein, 9 aa, Artificial sequence
  Synthetic sequence
CAEIVGHRC

SEQ ID NO: 219 - protein, 9 aa, Artificial sequence
  Synthetic sequence
CRVNHTSIC

SEQ ID NO: 220 - protein, 9 aa, Artificial sequence
  Synthetic sequence
CAEIHTGRC

SEQ ID NO: 221 - protein, 9 aa, Artificial sequence
  Synthetic sequence
CRVNHTSAC

SEQ ID NO: 222 - protein, 9 aa, Artificial sequence
  Synthetic sequence
CRVNHASQC

SEQ ID NO: 223 - protein, 9 aa, Artificial sequence
  Synthetic sequence
CVSNAQERC

SEQ ID NO: 224 - protein, 9 aa, Artificial sequence
  Synthetic sequence
CQVTEIANC

SEQ ID NO: 225 - protein, 9 aa, Artificial sequence
  Synthetic sequence
CHTNAVSQC

SEQ ID NO: 226 - protein, 9 aa, Artificial sequence
  Synthetic sequence
CQVNHFISC

SEQ ID NO: 227 - protein, 9 aa, Artificial sequence
  Synthetic sequence
CQVNFASTC

SEQ ID NO: 228 - protein, 9 aa, Artificial sequence
  Synthetic sequence
CHVQGENSC

SEQ ID NO: 229 - protein, 9 aa, Artificial sequence
  Synthetic sequence
CQRHVASTC

SEQ ID NO: 230 - protein, 9 aa, Artificial sequence
  Synthetic sequence
CTSRNQHVC

SEQ ID NO: 231 - protein, 9 aa, Artificial sequence
  Synthetic sequence
CRVSFHTQC

SEQ ID NO: 232 - protein, 9 aa, Artificial sequence
  Synthetic sequence
CQRITSHAC

SEQ ID NO: 233 - protein, 9 aa, Artificial sequence
  Synthetic sequence
CRAHFGESC

SEQ ID NO: 234 - protein, 9 aa, Artificial sequence
  Synthetic sequence
CAEIGRNSC

SEQ ID NO: 235 - protein, 9 aa, Artificial sequence
  Synthetic sequence
CTINHRVSC

SEQ ID NO: 236 - protein, 9 aa, Artificial sequence
  Synthetic sequence
CQVFATSHC

SEQ ID NO: 237 - protein, 9 aa, Artificial sequence
  Synthetic sequence
CSRVNTGQC

SEQ ID NO: 238 - protein, 9 aa, Artificial sequence
  Synthetic sequence
CTINHRSVC

SEQ ID NO: 239 - protein, 9 aa, Artificial sequence
  Synthetic sequence
CTIHSVQNC

SEQ ID NO: 240 - protein, 9 aa, Artificial sequence
  Synthetic sequence
CQVTAGRSC

SEQ ID NO: 241 - protein, 9 aa, Artificial sequence
  Synthetic sequence
CTNAIRFSC

SEQ ID NO: 242 - protein, 9 aa, Artificial sequence
  Synthetic sequence
CQVAGIHNC

SEQ ID NO: 243 - protein, 9 aa, Artificial sequence
  Synthetic sequence
CTIEGFANC

SEQ ID NO: 244 - protein, 9 aa, Artificial sequence
  Synthetic sequence
CTNFEGSRC

SEQ ID NO: 245 - protein, 9 aa, Artificial sequence
  Synthetic sequence
CQVNRASHC

SEQ ID NO: 246 - protein, 9 aa, Artificial sequence
  Synthetic sequence
CIVQANERC

SEQ ID NO: 247 - protein, 9 aa, Artificial sequence
  Synthetic sequence
CVFSNQITC

SEQ ID NO: 248 - protein, 9 aa, Artificial sequence
  Synthetic sequence
CVFSITGQC

SEQ ID NO: 249 - protein, 9 aa, Artificial sequence
  Synthetic sequence
CVSHTNRFC

SEQ ID NO: 250 - protein, 9 aa, Artificial sequence
  Synthetic sequence
CVSGFETAC

SEQ ID NO: 251 - protein, 9 aa, Artificial sequence
  Synthetic sequence
CRVAQTGIC

SEQ ID NO: 252 - protein, 9 aa, Artificial sequence
  Synthetic sequence
CVIRQSNTC

SEQ ID NO: 253 - protein, 9 aa, Artificial sequence
  Synthetic sequence
CVSETRNIC

SEQ ID NO: 254 - protein, 9 aa, Artificial sequence
  Synthetic sequence
CVNARISFC

SEQ ID NO: 255 - protein, 9 aa, Artificial sequence
  Synthetic sequence
CAEIFGQNC

SEQ ID NO: 256 - protein, 9 aa, Artificial sequence
  Synthetic sequence
CAERSGIVC

SEQ ID NO: 257 - protein, 9 aa, Artificial sequence
  Synthetic sequence
CAEHNISQC

SEQ ID NO: 258 - protein, 9 aa, Artificial sequence
  Synthetic sequence
CNGTGSHQC

SEQ ID NO: 259 - protein, 9 aa, Artificial sequence
  Synthetic sequence
CVSFINTQC

SEQ ID NO: 260 - protein, 9 aa, Artificial sequence
  Synthetic sequence
CSRQHNEFC

SEQ ID NO: 261 - protein, 9 aa, Artificial sequence
  Synthetic sequence
CQNFIERAC

SEQ ID NO: 262 - protein, 9 aa, Artificial sequence
  Synthetic sequence
CVSFGIENC

SEQ ID NO: 263 - protein, 9 aa, Artificial sequence
  Synthetic sequence
CVNTERFGC

SEQ ID NO: 264 - protein, 9 aa, Artificial sequence
  Synthetic sequence
CNSTAVQGC

SEQ ID NO: 265 - protein, 9 aa, Artificial sequence
  Synthetic sequence
CNSITERVC

SEQ ID NO: 266 - protein, 9 aa, Artificial sequence
  Synthetic sequence
CVSNEFGTC

SEQ ID NO: 267 - protein, 9 aa, Artificial sequence
  Synthetic sequence
CAEIQGNRC

SEQ ID NO: 268 - protein, 9 aa, Artificial sequence
  Synthetic sequence
CHSALTKHC

SEQ ID NO: 269 - protein, 9 aa, Artificial sequence
  Synthetic sequence
CMGPSSMLC

SEQ ID NO: 270 - protein, 9 aa, Artificial sequence
  Synthetic sequence
CTDPNQLQC

SEQ ID NO: 271 - protein, 9 aa, Artificial sequence
  Synthetic sequence
CSTHFIDTC

SEQ ID NO: 272 - protein, 5 aa, Artificial sequence
  Synthetic sequence
AAAAA

SEQ ID NO: 273 - protein, 5 aa, Artificial sequence
  Synthetic sequence
AAAAA

SEQ ID NO: 274 - protein, 5 aa, Artificial sequence
  Synthetic sequence
AAAAA

SEQ ID NO: 275 - protein, 5 aa, Artificial sequence
  Synthetic sequence
AAAAA

SEQ ID NO: 276 - protein, 5 aa, Artificial sequence
  Synthetic sequence
AAAAA

SEQ ID NO: 277 - protein, 5 aa, Artificial sequence
  Synthetic sequence
AAAAA

SEQ ID NO: 278 - protein, 5 aa, Artificial sequence
  Synthetic sequence
AAAAA

SEQ ID NO: 279 - protein, 5 aa, Artificial sequence
  Synthetic sequence
AAAAA

SEQ ID NO: 280 - protein, 5 aa, Artificial sequence
  Synthetic sequence
AAAAA

SEQ ID NO: 281 - protein, 5 aa, Artificial sequence
  Synthetic sequence
AAAAA

SEQ ID NO: 282 - protein, 5 aa, Artificial sequence
  Synthetic sequence
AAAAA

SEQ ID NO: 283 - protein, 5 aa, Artificial sequence
  Synthetic sequence
AAAAA

SEQ ID NO: 284 - protein, 5 aa, Artificial sequence
  Synthetic sequence
AAAAA

SEQ ID NO: 285 - protein, 5 aa, Artificial sequence
  Synthetic sequence
AAAAA

SEQ ID NO: 286 - protein, 5 aa, Artificial sequence
  Synthetic sequence
AAAAA

SEQ ID NO: 287 - protein, 5 aa, Artificial sequence
  Synthetic sequence
AAAAA

SEQ ID NO: 288 - protein, 5 aa, Artificial sequence
  Synthetic sequence
AAAAA

SEQ ID NO: 289 - protein, 5 aa, Artificial sequence
  Synthetic sequence
AAAAA

SEQ ID NO: 290 - protein, 5 aa, Artificial sequence
  Synthetic sequence
AAAAA

SEQ ID NO: 291 - protein, 5 aa, Artificial sequence
  Synthetic sequence
AAAAA

SEQ ID NO: 292 - protein, 5 aa, Artificial sequence
  Synthetic sequence
AAAAA

SEQ ID NO: 293 - protein, 5 aa, Artificial sequence
  Synthetic sequence
AAAAA

SEQ ID NO: 294 - protein, 5 aa, Artificial sequence
  Synthetic sequence
AAAAA

SEQ ID NO: 295 - protein, 5 aa, Artificial sequence
  Synthetic sequence
AAAAA

SEQ ID NO: 296 - protein, 5 aa, Artificial sequence
  Synthetic sequence
AAAAA

SEQ ID NO: 297 - protein, 5 aa, Artificial sequence
  Synthetic sequence
AAAAA

SEQ ID NO: 298 - protein, 5 aa, Artificial sequence
  Synthetic sequence
AAAAA

SEQ ID NO: 299 - protein, 5 aa, Artificial sequence
  Synthetic sequence
AAAAA

SEQ ID NO: 300 - protein, 5 aa, Artificial sequence
  Synthetic sequence
AAAAA

SEQ ID NO: 301 - protein, 7 aa, Artificial sequence
  Synthetic sequence
TGSTQHQ

SEQ ID NO: 302 - protein, 5 aa, Artificial sequence
  Synthetic sequence
SHNHT

SEQ ID NO: 303 - protein, 6 aa, Artificial sequence
  Synthetic sequence
THTGRN

SEQ ID NO: 304 - protein, 7 aa, Artificial sequence
  Synthetic sequence
SATLQHS

SEQ ID NO: 305 - protein, 8 aa, Artificial sequence
  Synthetic sequence
NAHQARST

SEQ ID NO: 306 - protein, 7 aa, Artificial sequence
  Synthetic sequence
SLTVNWN

SEQ ID NO: 307 - protein, 7 aa, Artificial sequence
  Synthetic sequence
LSVNHNA

SEQ ID NO: 308 - protein, 7 aa, Artificial sequence
  Synthetic sequence
SASTNHN

SEQ ID NO: 309 - protein, 7 aa, Artificial sequence
  Synthetic sequence
SASQVHN

SEQ ID NO: 310 - protein, 7 aa, Artificial sequence
  Synthetic sequence
NGTGSHQ

SEQ ID NO: 311 - protein, 7 aa, Artificial sequence
  Synthetic sequence
SVTTQHQ

SEQ ID NO: 312 - protein, 7 aa, Artificial sequence
  Synthetic sequence
VSVTNHQ

SEQ ID NO: 313 - protein, 7 aa, Artificial sequence
  Synthetic sequence
TSTGRNA

SEQ ID NO: 314 - protein, 7 aa, Artificial sequence
  Synthetic sequence
KTGSHNQ

SEQ ID NO: 315 - protein, 5 aa, Artificial sequence
  Synthetic sequence
AAAAA

SEQ ID NO: 316 - protein, 5 aa, Artificial sequence
  Synthetic sequence
AAAAA

SEQ ID NO: 317 - protein, 5 aa, Artificial sequence
  Synthetic sequence
AAAAA

SEQ ID NO: 318 - protein, 5 aa, Artificial sequence
  Synthetic sequence
AAAAA

SEQ ID NO: 319 - protein, 5 aa, Artificial sequence
  Synthetic sequence
AAAAA

SEQ ID NO: 320 - protein, 5 aa, Artificial sequence
  Synthetic sequence
AAAAA

SEQ ID NO: 321 - protein, 5 aa, Artificial sequence
  Synthetic sequence
AAAAA

SEQ ID NO: 322 - protein, 5 aa, Artificial sequence
  Synthetic sequence
AAAAA

SEQ ID NO: 323 - protein, 7 aa, Artificial sequence
  Synthetic sequence
SSSPSKH

SEQ ID NO: 324 - protein, 5 aa, Artificial sequence
  Synthetic sequence
AAAAA

SEQ ID NO: 325 - protein, 5 aa, Artificial sequence
  Synthetic sequence
AAAAA

SEQ ID NO: 326 - protein, 7 aa, Artificial sequence
  Synthetic sequence
TIQHRAE

SEQ ID NO: 327 - protein, 7 aa, Artificial sequence
  Synthetic sequence
TIQHGRS

SEQ ID NO: 328 - protein, 7 aa, Artificial sequence
  Synthetic sequence
VSAGQNH

SEQ ID NO: 329 - protein, 7 aa, Artificial sequence
  Synthetic sequence
VSIGNHN

SEQ ID NO: 330 - protein, 7 aa, Artificial sequence
  Synthetic sequence
VSANHQI

SEQ ID NO: 331 - protein, 7 aa, Artificial sequence
  Synthetic sequence
VSATGNH

SEQ ID NO: 332 - protein, 7 aa, Artificial sequence
  Synthetic sequence
AEGINVH

SEQ ID NO: 333 - protein, 7 aa, Artificial sequence
  Synthetic sequence
VSEHING

SEQ ID NO: 334 - protein, 7 aa, Artificial sequence
  Synthetic sequence
TIQHRAF

SEQ ID NO: 335 - protein, 7 aa, Artificial sequence
  Synthetic sequence
RVAHFIT

SEQ ID NO: 336 - protein, 7 aa, Artificial sequence
  Synthetic sequence
VSEQHNI

SEQ ID NO: 337 - protein, 7 aa, Artificial sequence
  Synthetic sequence
VSANHQT

SEQ ID NO: 338 - protein, 7 aa, Artificial sequence
  Synthetic sequence
TSVINEH

SEQ ID NO: 339 - protein, 7 aa, Artificial sequence
  Synthetic sequence
AEHRSQT

SEQ ID NO: 340 - protein, 7 aa, Artificial sequence
  Synthetic sequence
RVTNHQS

SEQ ID NO: 341 - protein, 7 aa, Artificial sequence
  Synthetic sequence
TIHNRQS

SEQ ID NO: 342 - protein, 7 aa, Artificial sequence
  Synthetic sequence
VSENHQG

SEQ ID NO: 343 - protein, 7 aa, Artificial sequence
  Synthetic sequence
VSAGNHQ

SEQ ID NO: 344 - protein, 7 aa, Artificial sequence
  Synthetic sequence
RVAIHGN

SEQ ID NO: 345 - protein, 7 aa, Artificial sequence
  Synthetic sequence
VSATQHN

SEQ ID NO: 346 - protein, 7 aa, Artificial sequence
  Synthetic sequence
VSATFNH

SEQ ID NO: 347 - protein, 7 aa, Artificial sequence
  Synthetic sequence
THNRQSF

SEQ ID NO: 348 - protein, 7 aa, Artificial sequence
  Synthetic sequence
IEVNHNR

SEQ ID NO: 349 - protein, 7 aa, Artificial sequence
  Synthetic sequence
VSEFNTH

SEQ ID NO: 350 - protein, 7 aa, Artificial sequence
  Synthetic sequence
HISGVFN

SEQ ID NO: 351 - protein, 7 aa, Artificial sequence
  Synthetic sequence
IEVNHNS

SEQ ID NO: 352 - protein, 7 aa, Artificial sequence
  Synthetic sequence
HISGEAR

SEQ ID NO: 353 - protein, 7 aa, Artificial sequence
  Synthetic sequence
IVNHFRQ

SEQ ID NO: 354 - protein, 7 aa, Artificial sequence
  Synthetic sequence
HSAGIFV

SEQ ID NO: 355 - protein, 7 aa, Artificial sequence
  Synthetic sequence
IEVNHQS

SEQ ID NO: 356 - protein, 7 aa, Artificial sequence
  Synthetic sequence
VIHFTRN

SEQ ID NO: 357 - protein, 7 aa, Artificial sequence
  Synthetic sequence
GVQHSRN

SEQ ID NO: 358 - protein, 7 aa, Artificial sequence
  Synthetic sequence
VSNQIER

SEQ ID NO: 359 - protein, 7 aa, Artificial sequence
  Synthetic sequence
VSTGNHN

SEQ ID NO: 360 - protein, 7 aa, Artificial sequence
  Synthetic sequence
THENRQS

SEQ ID NO: 361 - protein, 7 aa, Artificial sequence
  Synthetic sequence
VSAFTHG

SEQ ID NO: 362 - protein, 7 aa, Artificial sequence
  Synthetic sequence
IEANTFH

SEQ ID NO: 363 - protein, 7 aa, Artificial sequence
  Synthetic sequence
AEQGHTR

SEQ ID NO: 364 - protein, 7 aa, Artificial sequence
  Synthetic sequence
TQVNHRS

SEQ ID NO: 365 - protein, 7 aa, Artificial sequence
  Synthetic sequence
AVRENQT

SEQ ID NO: 366 - protein, 7 aa, Artificial sequence
  Synthetic sequence
TIANRQS

SEQ ID NO: 367 - protein, 7 aa, Artificial sequence
  Synthetic sequence
TIVNHRS

SEQ ID NO: 368 - protein, 7 aa, Artificial sequence
  Synthetic sequence
AEIVGHR

SEQ ID NO: 369 - protein, 7 aa, Artificial sequence
  Synthetic sequence
RVNHTSI

SEQ ID NO: 370 - protein, 7 aa, Artificial sequence
  Synthetic sequence
AEIHTGR

SEQ ID NO: 371 - protein, 7 aa, Artificial sequence
  Synthetic sequence
RVNHTSA

SEQ ID NO: 372 - protein, 7 aa, Artificial sequence
  Synthetic sequence
RVNHASQ

SEQ ID NO: 373 - protein, 7 aa, Artificial sequence
  Synthetic sequence
VSNAQER

SEQ ID NO: 374 - protein, 7 aa, Artificial sequence
  Synthetic sequence
QVTEIAN

SEQ ID NO: 375 - protein, 7 aa, Artificial sequence
  Synthetic sequence
HTNAVSQ

SEQ ID NO: 376 - protein, 7 aa, Artificial sequence
  Synthetic sequence
QVNHFIS

SEQ ID NO: 377 - protein, 7 aa, Artificial sequence
  Synthetic sequence
QVNFAST

SEQ ID NO: 378 - protein, 7 aa, Artificial sequence
  Synthetic sequence
HVQGENS

SEQ ID NO: 379 - protein, 7 aa, Artificial sequence
  Synthetic sequence
QRHVAST

SEQ ID NO: 380 - protein, 7 aa, Artificial sequence
  Synthetic sequence
TSRNQHV

SEQ ID NO: 381 - protein, 7 aa, Artificial sequence
  Synthetic sequence
RVSFHTQ

SEQ ID NO: 382 - protein, 7 aa, Artificial sequence
  Synthetic sequence
QRITSHA

SEQ ID NO: 383 - protein, 7 aa, Artificial sequence
  Synthetic sequence
RAHFGES

SEQ ID NO: 384 - protein, 7 aa, Artificial sequence
  Synthetic sequence
AEIGRNS

SEQ ID NO: 385 - protein, 7 aa, Artificial sequence
  Synthetic sequence
TINHRVS

SEQ ID NO: 386 - protein, 7 aa, Artificial sequence
  Synthetic sequence
QVFATSH

SEQ ID NO: 387 - protein, 7 aa, Artificial sequence
  Synthetic sequence
SRVNTGQ

SEQ ID NO: 388 - protein, 7 aa, Artificial sequence
  Synthetic sequence
TINHRSV

SEQ ID NO: 389 - protein, 7 aa, Artificial sequence
  Synthetic sequence
TIHSVQN

SEQ ID NO: 390 - protein, 7 aa, Artificial sequence
  Synthetic sequence
QVTAGRS

SEQ ID NO: 391 - protein, 7 aa, Artificial sequence
  Synthetic sequence
TNAIRFS

SEQ ID NO: 392 - protein, 7 aa, Artificial sequence
  Synthetic sequence
QVAGIHN

SEQ ID NO: 393 - protein, 7 aa, Artificial sequence
  Synthetic sequence
TIEGFAN

SEQ ID NO: 394 - protein, 7 aa, Artificial sequence
  Synthetic sequence
TNFEGSR

SEQ ID NO: 395 - protein, 7 aa, Artificial sequence
  Synthetic sequence
QVNRASH

SEQ ID NO: 396 - protein, 7 aa, Artificial sequence
  Synthetic sequence
IVQANER

SEQ ID NO: 397 - protein, 7 aa, Artificial sequence
  Synthetic sequence
VFSNQIT

SEQ ID NO: 398 - protein, 7 aa, Artificial sequence
  Synthetic sequence
VFSITGQ

SEQ ID NO: 399 - protein, 7 aa, Artificial sequence
  Synthetic sequence
VSHTNRF

SEQ ID NO: 400 - protein, 7 aa, Artificial sequence
  Synthetic sequence
VSGFETA

SEQ ID NO: 401 - protein, 7 aa, Artificial sequence
  Synthetic sequence
RVAQTGI

SEQ ID NO: 402 - protein, 7 aa, Artificial sequence
  Synthetic sequence
VIRQSNT

SEQ ID NO: 403 - protein, 7 aa, Artificial sequence
  Synthetic sequence
VSETRNI

SEQ ID NO: 404 - protein, 7 aa, Artificial sequence
  Synthetic sequence
VNARISF

SEQ ID NO: 405 - protein, 7 aa, Artificial sequence
  Synthetic sequence
AEIFGQN

SEQ ID NO: 406 - protein, 7 aa, Artificial sequence
  Synthetic sequence
AERSGIV

SEQ ID NO: 407 - protein, 7 aa, Artificial sequence
  Synthetic sequence
AEHNISQ

SEQ ID NO: 408 - protein, 7 aa, Artificial sequence
  Synthetic sequence
NGTGSHQ

SEQ ID NO: 409 - protein, 7 aa, Artificial sequence
  Synthetic sequence
VSFINTQ

SEQ ID NO: 410 - protein, 7 aa, Artificial sequence
  Synthetic sequence
SRQHNEF

SEQ ID NO: 411 - protein, 7 aa, Artificial sequence
  Synthetic sequence
QNFIERA

SEQ ID NO: 412 - protein, 7 aa, Artificial sequence
  Synthetic sequence
VSFGIEN

SEQ ID NO: 413 - protein, 7 aa, Artificial sequence
  Synthetic sequence
VNTERFG

SEQ ID NO: 414 - protein, 7 aa, Artificial sequence
  Synthetic sequence
NSTAVQG

SEQ ID NO: 415 - protein, 7 aa, Artificial sequence
  Synthetic sequence
NSITERV

SEQ ID NO: 416 - protein, 7 aa, Artificial sequence
  Synthetic sequence
VSNEFGT

SEQ ID NO: 417 - protein, 7 aa, Artificial sequence
  Synthetic sequence
AEIQGNR

SEQ ID NO: 418 - protein, 7 aa, Artificial sequence
  Synthetic sequence
HSALTKH

SEQ ID NO: 419 - protein, 7 aa, Artificial sequence
  Synthetic sequence
MGPSSML

SEQ ID NO: 420 - protein, 7 aa, Artificial sequence
  Synthetic sequence
TDPNQLQ

SEQ ID NO: 421 - protein, 7 aa, Artificial sequence
  Synthetic sequence
STHFIDT

(19) United States
(12) Patent Application Publication
(10) Pub. No.: US 2018/0251495 A1
(43) Pub. Date: Sep. 6, 2018

(54) Skin-Penetrating Peptides and Compositions and Methods of Use Thereof

(71) Applicant: The Regents of the University of California, Oakland, CA (US)

(72) Inventors: Samir M. Mitragotri, Santa Barbara, CA (US); Stefano Menegatti, Aliso Viejo, CA (US)

(73) Assignee: The Regents of the University of California, Oakland, CA (US)

(21) Appl. No.: 15/760,183

(22) PCT Filed: Sep. 14, 2016
(86) PCT No.: PCT/US2016/051688

§ 371 Date: Mar. 14, 2018

Related U.S. Application Data
(60) Provisional application No. 62/218,621, filed on Sep. 15, 2015.

(51) Int. Cl. C07K 7/64; A61K 47/64; A61K 9/00

(52) U.S. Cl. CPC C07K 7/64; C07K 7/645; A61K 38/00; A61K 9/0014; A61K 47/64

(57) ABSTRACT

Skin penetrating polypeptide and pharmaceutical compositions and methods of use thereof are provided. Typically, the peptides are between 3 and 100 amino acids, more preferably about 5, 6, 7, 8, 9, or 10 amino acids, cyclic, binds to a skin protein such as a keratin with a Kd of between about 10-3 M and about 10-8 M. Preferably the peptides increase absorption or penetration of an active agent into the skin. In silico methods of screening for skin protein binding polypeptides are also provided.

### CROSS-REFERENCE

This application claims the benefit of U.S. Provisional Patent Application No. 62/218,621 filed Sep. 15, 2015, which application is incorporated herein by reference in its entirety.

### INCORPORATION BY REFERENCE OF SEQUENCE LISTING PROVIDED AS A TEXT FILE

A Sequence Listing is provided herewith as a text file, “UCSB-544WO-SeqList_ST25.txt” created on Aug. 2, 2016 and having a size of 86 KB. The contents of the text file are incorporated by reference herein in their entirety.

### INTRODUCTION

Transdermal drug delivery is a growing area within the field of drug delivery. Transdermal delivery offers numerous advantages over traditional techniques, especially due to the elimination of needles, which reduces both risk of infections and discomfort in patients. See, e.g., Barry, B. W., Eur J Pharm Sci, 14(2):101-114 (2001); Brown, et al., Methods Mol Biol, 437:119-139 (2008); Dhote, et al., Sci Pharm, 80(1):1-28 (2012); Delgado-Charro, et al., Adv Drug Deliv Rev, 73:63-82 (2014). Drug permeation into and across skin, however, still poses serious challenges, mainly related to the natural imperviousness of this tissue. See, e.g., Tiwary, et al., Recent Pat Drug Deliv Formul, 1(1):23-36 (2007); Rizwan, et al., Recent Pat Drug Deliv Formul, 3(2):105-24 (2009). Among the various skin layers, the stratum corneum (SC) is particularly important in protecting underlying organs from foreign agents, such as pathogens and toxins. SC includes keratin-rich cells embedded in multiple lipid bilayers. See, e.g. Norlen, et al., J Investig Dermatol, 123(4):715-732 (2004). The hydrophobicity and the densely packed structure of this layer render particularly difficult the permeation of even small therapeutically active ingredients Morgan, et al., Br J Dermatol, 148(3) 434-443 (2003). To increase drug permeation across the tissue, chemical permeation enhancers (CPEs) have been proposed, including small synthetic chemicals (azone derivatives, fatty acids, alcohols, esters, sulfoxides, pyrrolidones, glycols, surfactants and terpenes) and peptides. Williams, et al., Adv Drug Deliv Rev, 56(5):603-818 (2004); Whitehead, et al., J Control Release, 128(2):128-133 (2008); Mittal, et al., Curr Drug Deliv, 6(3):274-279 (2009); El Maghraby, et al., Saudi Pharm J, 23(1)67-74 (2015).

Compositions and methods for transdermal drug delivery are discussed in U.S. Pat. Nos. 5,814,599, 5,947,921, 6,002,961, 6,018,678, 6,041,253, 6,190,315, 6,234,990, 6,491,657, 6,620,123, 7,795,309, 8,021,323, 8,277,762, 8,287,483, 8,343,962, 8,513,304, 8,518,871, and 8,791,062. A number of small (1,000-1,500 Da) skin penetrating peptides for the transdermal delivery of highly relevant drug models, such as siRNA, hyaluronic acid and Cyclosporine A (CsA) are discussed in Hsu, et al., Proc Natl Acad Sci USA, 108(38):15816-15821 (2011). Several fundamental aspects underlying the mechanism of skin permeation enhancement by peptides were discussed in Kumar, et al., J Control Release, 199:168-178 (2015). Findings obtained for five sequences (SPACE™, TD-1, Poly-R, DLP and LP-12) with different physicochemical properties, indicates that SPP's mechanism of transport occurs mainly throughout the intracellular (or transcellular) matrix. This is supported by experimental observations of the structural alteration by SPPs of the proteins of the stratum corneum, as well as by affinity binding studies that indicate an affinity between SPPs and keratin, the most abundant skin protein.

There remains a need for additional skin penetrating peptides (e.g., peptides that can provide for enhanced absorption/penetration of an active agent of interest into the skin), pharmaceutical compositions including skin penetrating peptides and an active agent of interest, methods of screening for skin penetrating peptides, and methods of treating a subject using skin penetrating peptides. These needs are addressed herein.

### SUMMARY

Skin penetrating polypeptides are provided. Typically, the peptides include or consist of between about 3 and about 100 amino acids, and bind to a skin protein with a Kd of between about 10⁻³ M and about 10⁻⁸ M. In preferred embodiments, the skin protein is keratin, collagen, plectin, actin, or tubulin. In the most preferred embodiments, the skin protein is one, two or more keratins, for example, keratin 5, keratin 14, or a combination thereof.

The polypeptide can include or consists of Cys-X1-Xn-Cys (e.g., SEQ ID NO:16), or Ala - Cys-X1-Xn-Cys-Gly (e.g., SEQ ID NO:17), wherein each “X” is independently any amino acid, or a subset thereof, for example the 19 canonical amino acids excluding cysteine; wherein “n” is 0 or an integer between 1 and 100 inclusive; and wherein peptide cyclization is achieved through the formation of a disulfide bond between two cysteines, for example the two cysteines framing the sequence. In particular embodiments, “n” is 0, 1, 2, 3, 4, 5, 6, 7, 8, 9, or 10. Preferably, the amino acid sequence includes one or more sequence motifs selected from the group consisting of NHN, QHN, NRN, and QRQ.

In some embodiments, the polypeptide includes or consists of the amino acid sequence cyclo[C-X1-X2-X3-X4-X5-C] (SEQ ID NO:18), wherein “C” is a Cysteine; “X1” is Serine, Threonine, Asparagine, Glutamine, or Glycine; “X2” is Histidine, Asparagine, or Glutamine; “X3” is Histidine, Arginine, Asparagine, or Glutamine; “X4” is Histidine, Asparagine, Glutamine, Serine, or Threonine; “X5” is Serine, Threonine, Glycine, or Alanine; and wherein peptide cyclization is achieved through the formation of a disulfide bond between two cysteines, for example the two cysteines framing the sequence (e.g., “C” of SEQ ID NO:18).

The peptide can include or consist of the amino acid sequence: cyclo[C-X1-X2-X3-X4-X5-X6-C] (SEQ ID NO:19), wherein “C” is Cysteine; “X1” is Serine, Threonine, Asparagine, Glutamine, or Glycine; “X2” is Serine, Threonine, Asparagine, or Glutamine; “X3” is Histidine, Arginine, Lysine, Asparagine, Glutamine, Glycine, or Alanine; “X4” is Serine, Threonine, Asparagine, Glutamine, Glycine, or Arginine; “X5” is Histidine, Arginine, Lysine, Asparagine, Glutamine, Serine, or Threonine; “X6” is Asparagine, Glutamine, Serine, Threonine, Arginine, Glycine, or Alanine; and wherein peptide cyclization is achieved through the formation of a disulfide bond between two cysteines framing the sequence, for example the two cysteines framing the sequence (e.g., “C” of SEQ ID NO:19).

The polypeptide can include or consist of the amino acid sequence cyclo[C—X1-X2-X3-X4-X5-X6-X7-C] (SEQ ID NO:20), wherein “C” is a Cysteine; “X1” is Serine, Threonine, Glycine, Alanine, or Valine; “X2” is Glycine, Alanine, Valine, Leucine, Serine, or Threonine; “X3” is Glycine, Alanine, Serine, or Threonine; “X4” is Asparagine, Glutamine, Arginine, or Lysine; “X5” is Histidine, Asparagine, Glutamine, Tryptophan, Serine, or Threonine; “X6” is Serine, Threonine, Histidine, Asparagine, Glutamine, Glycine, or Alanine; “X7” is Serine, Threonine, Histidine, Asparagine, Glutamine, Glycine, or Alanine; and wherein peptide cyclization is achieved through the formation of a disulfide bond between two cysteines, for example the two cysteines framing the sequence (e.g., “C” of SEQ ID NO:20)

The polypeptide can include or consist of the amino acid sequence cyclo[C—X1-X2-X3-X4-X5-X6-X7-X8-C] (SEQ ID NO:21), wherein “C” is a Cysteine; “X1” is Serine, Threonine, Asparagine, Glutamine, Glycine, or Alanine; “X2” is Alanine, Serine, Threonine, or Arginine; “X3” is Histidine, Asparagine, Glutamine, Lysine, or Arginine; “X4” is Asparagine, Arginine, Histidine, or Tryptophan; “X5” is Glycine, Alanine, Arginine, Glutamine, Lysine, or Arginine; “X6” is Histidine, Tryptophan, Glycine, or Alanine; “X7” is Serine, Threonine, Asparagine, or Glutamine; “X8” is Serine, Threonine, Glycine, or Alanine; and wherein peptide cyclization is achieved through the formation of a disulfide bond between two cysteines , for example the two cysteines framing the sequence (e.g., “C” of SEQ ID NO:21).

In specific embodiments, the polypeptide includes or consists of the amino acid sequence of SEQ ID NO: 2, 3, 4, 5, 6, 7, 8, 9, 10, 11, 12, or 13. In some embodiments, the peptide does not include or consist of SEQ ID NO:1 (SPACE™), SEQ ID NO:23 (TD-1), SEQ ID NO:24 (Poly-R), SEQ ID NO:14 (DLP), or SEQ ID NO:25 (LP-12).

Preferably, the polypeptide binds to an active agent. The peptide can increase absorption or penetration of the active agent into one or more tissues or cells of the skin compared to absorption or penetration of the active agent in the absence of the peptide, when the peptide and the active agent are administered in combination to the skin of a mammalian subject. For example, in some embodiments, the peptide can increase delivery of the active agent across the stratum corneum when the peptide and the active agent are administered in combination to the skin of a mammalian subject. In specific embodiments, the active agent is Cyclosporine A.

Pharmaceutical compositions include the disclosed skin penetrating polypeptides, and optionally including an active agent are also provided.

Methods of using the polypeptides are also disclosed. In some embodiments, the methods include treating a subject in need thereof by administering to the subject a skin penetrating polypeptide in combination with an active agent. The polypeptide and the active agent can be together in the same pharmaceutical composition or in different pharmaceutical composition. In preferred embodiments, the polypeptide and active agent are administered topically, for example to the skin of the subject. Preferably, the polypeptide is administered in an effective amount to increase absorption or penetration of the active agent into the skin. For example, in some embodiments, the peptide increases delivery of the active agent across the stratum corneum, compared to administering the subject the active agent in the absence of the peptide.

The active agent can be, for example, a polypeptide, nucleic acid, or a small molecule. In some embodiments, the active agent is a dermatological agent. In a particular embodiment, the active agent is cyclosporine A. The subject can have a dermatological condition, disease, or disorder. For example, in some embodiments, the active agent is administered to the subject in an effective amount reduce one or symptoms associated with the dermatological condition, disease, or disorder.

Methods of screening for skin penetrating peptides in silico are also provided. Typically the methods include screening a virtual peptide library for binding to a skin protein by individually simulating binding of the active residues of each peptide's crystal structure to the active residues of the skin protein's residues, and selecting the peptide as a skin penetrating peptide if the predicted dissociation constant (Kd) is between about 10⁻³ M and 10⁻⁸ M.

The peptide library can include, for example, randomization of the sequences X1-Xn (wherein each “X” is independently any amino acid, or a specific sub-set thereof, and wherein “n” is an integer between 2 and 100 inclusive). In particular embodiments, the skin protein is selected from the group consisting of keratin, collagen, plectin, actin, and tubulin. In some embodiments, the polypeptides of the peptide library include or consist of Cys-X1-Xn-Cys (SEQ ID NO:16), or Ala-Cys-X1-Xn-Cys-Gly (SEQ ID NO:17), wherein each “X” is independently any amino acid, or a subset thereof for example the 19 canonical amino acids excluding cysteine; wherein “n” is 0 or an integer between 1 and 100 inclusive; and wherein peptide cyclization is achieved through the formation of a disulfide bond between two cysteines. In some embodiments, is 0, 1, 2, 3, 4, 5, 6, 7, 8, 9, or 10, or a combination thereof In some embodiments, the peptide library excludes peptides that have less than “z” different amino acids; more than two consecutive equal amino acids; more than “z” aliphatic amino acids (Ala, Val, Leu, and Ile); “z” aromatic amino acids (Phe, Tyr, and Trp); less than “z” charged amino acids (Lys, Arg, His, Asp, and Glu); more than “z” charged amino acids (Lys, Arg, His, Asp, and Glu); only alternated hydrophobic and charged amino acids; or any combination thereof and wherein “z” is an integer between 2 and 100 inclusive, but longer than the “n” length of the peptide.

The method can alternatively or additionally include screening the peptides for binding to active agent by individually simulating binding of the active residues of each peptide's crystal structure to the active residues of the active agent's residues, and selecting the peptide as a skin penetrating peptide if the predicted dissociation constant (Kd) is between about 10⁻³ M and 10⁻⁸ M. The active residues of the skin protein can be, for example, those that exhibit a relative solvent accessibility higher than 40%, as defined by the program NACCESS. In some embodiments, all of the residues of each peptide in the peptide library are active residues.

### BRIEF DESCRIPTION OF THE DRAWINGS

FIG. 1 is a spectrogram showing mass spectrometric analysis of SP7-1-CsA complex in solution.

FIG. 2 is a curve showing the Keratin binding isotherm of SP7-1 Q (mg/mL res) as a function of C (mg/mL).

FIG. 3 is a bar graph showing in vitro skin penetration of Cyclosporine A (CsA). CsA (5 mg/mL) in 45% (v/v) Ethanol/PBS (Control) or with selected SPPs in 45% (v/v) Ethanol/PBS were applied to the donor compartment of Franz-diffusion cells. The non-binding heptamer (ACGSGSGSGCG (SEQ ID NO:15)) was used as a second negative control. The amount of CsA entering the skin was determined for different skin layers. Each data point represents mean ±stdev (n=3) except for SPACE™ (SEQ ID NO:1) peptide (n=6). *p<0.05, indicates significance relative to SPACE™ (SEQ ID NO:1) peptide for that layer.

FIG. 4A is aline graph showing the % Viability (relative to control) of human epidermal keratinocytes after incubation with skin penetrating peptides (concentration (mg/ml)). SPACE™ (SEQ ID NO:1) (closed square), SP7-1 (SEQ ID NO:4) (closed circle), SP7-2 (SEQ ID NO:6) (closed triangle), SP7-3 (SEQ ID NO:7) (closed diamond), SP7-4 (SEQ ID NO:13) (open square), SP7-5 (SEQ ID NO:8) (open circle), SP6-1 (SEQ ID NO:3) (open triangle), SP8-1 (SEQ ID NO:5) (open diamond). Error bars represent mean±SD for n=3. All points below stars are significantly different relative to incubation with media alone.

FIG. 4B is a bar graph showing the % Viability of human epidermal keratinocytes at 5 mg/ml (same data as in FIG. 4A, replotted). * indicates data points significantly different compared to incubation with media alone.

FIG. 5A is a curve showing the Keratin binding isotherm of SPACE Q (mg/mL res) as a function of C (mg/mL).

FIG. 5B is a curve showing the Keratin binding isotherm of SP7-2 Q (mg/mL res) as a function of C (mg/mL).

FIG. 5C is a curve showing the Keratin binding isotherm of SP7-3 Q (mg/mL res) as a function of C (mg/mL).

FIG. 5D is a curve showing the Keratin binding isotherm of SP7-4 Q (mg/mL res) as a function of C (mg/mL).

FIG. 5E is a curve showing the Keratin binding isotherm of SP7-5 Q (mg/mL res) as a function of C (mg/mL).

### DETAILED DESCRIPTION

### I. Definitions

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “polypeptide” and “peptide” are used interchangeably and refer to a chain of amino acids of any length, regardless of modification (e.g., phosphorylation or glycosylation). The terms include proteins and fragments thereof. The polypeptides or peptides can be “exogenous,” meaning that they are “heterologous,” i.e., foreign to the host cell being utilized, such as human polypeptide produced by a bacterial cell. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity; particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0 ±1); glutamate (+3.0 ±1); serine (+0.3); asparagine (+0.2); glutamine (+0.2); glycine (0); proline (−0.5 ±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more sequence identity to the polypeptide of interest.

As used herein, the term “identity,” as known in the art, is a relationship between two or more polypeptide sequences, as determined by comparing the sequences. In the art, “identity” also means the degree of sequence relatedness between polypeptide as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and) (BLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein “treatment” or “treating” means to administer a composition to a subject or a system with an undesired condition (e.g., restenosis or other vascular proliferative disorder). The condition can include a disease. “Prevention” or “preventing” means to administer a composition to a subject or a system at risk for the condition. The condition can include a predisposition to a disease. The effect of the administration of the composition to the subject (either treating and/or preventing) can be, but is not limited to, the cessation of a particular symptom of a condition, a reduction or prevention of the symptoms of a condition, a reduction in the severity of the condition, the complete ablation of the condition, a stabilization or delay of the development or progression of a particular event or characteristic, or minimization of the chances that a particular event or characteristic will occur. It is understood that where treat or prevent are used, unless specifically indicated otherwise, the use of the other word is also expressly disclosed.

As used herein “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

As used herein, “operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

As used herein, a “graft” is a tissue for transplantation. This may be in the form of cells or non-dissociated tissue. It may or may not have been treated prior to implantation to sterilize, modify, or cleanse the graft. Grafts include autograft, allograft, and synthetic tissues and organs, tissues produced by tissue engineering and non-biological medical devices by attachment of specific ligands (i.e. counter ligands attached to each surface) or by electrostatic or other non-covalent means.

As used herein, “microparticles” refers to particles having a diameter between one micron and 1000 microns, typically less than 400 microns, more typically less than 100 microns, most preferably for the uses described herein in the range of less than 10 microns in diameter. Microparticles include microcapsules and microspheres unless otherwise specified.

As used herein, “nanoparticles” refer to particles having a diameter of less than one micron, more typically between 50 and 1000 nanometers, preferably in the range of 100 to 300 nanometers.

As used herein, the phrase that a molecule “specifically binds”, “bind specifically” or “displays specific binding” to a target refers to a binding reaction which is determinative of the presence of the molecule in the presence of a heterogeneous population of other biologics. It is preferred that such molecules bind the target molecule with a dissociation constant (K_{d}) less than or equal to 10⁻³, 10⁻⁴ 10⁻⁶, 10⁻⁸, 10⁻⁹, 10⁻¹⁰, 10⁻¹¹, or 10⁻¹².

As used herein, “dissociation constant (K_{d} or K_{D})” is an equilibrium constant that measures the propensity of one binding partner to separate reversibly from another binding partner, as when a complex falls apart into its component molecules, or when a salt splits up into its component ions. KD is the equilibrium dissociation constant, a ratio of koff/kon. The dissociation constant is the inverse of the association constant. For the bimolecular reaction, A+B⇔AB, the equilibrium dissociation constant (K_{d}) and equilibrium association constant (Kₐ) are reciprocally related,

For bimolecular reactions, the units of Kd are concentration (M, mM, etc.) and the units of Ka are concentration-1 (M-1, mM-1, μM-1, etc.).

### II. Compositions

A. Skin Penetrating Peptides

Skin penetrating peptides are provided. The disclosed skin penetrating peptides can (1) bind, preferably specifically, to a skin protein; (2) bind, preferably specifically, to an active agent; (3) increase the absorption or penetration of the active into or through the skin, preferably the stratum corneum; (4) exhibit little or no skin toxicity; or a combination thereof.

In some embodiments, the peptide does not include or consist of the amino acids sequence of ACTGSTQHQCG (SEQ ID NO:1) (“SPACE™”), ACSSSPSKHCG (SEQ ID NO:23) (“TD-1”), RRRRRRR (SEQ ID NO:24) (“Poly-R”), ACKTGSHNQCG (SEQ ID NO:14) (“DLP”), or HIITDPNMAEYL (SEQ ID NO:25) (“LP-12”).

1. Preferred Structural and Functional Elements

Typically the skin penetrating peptides have “n” amino acids, wherein “n” is an integer between 2 and 100 inclusive. The peptides are most typically between about 3 and 30 amino acids (inclusive) in length, preferably between about 4 and about 20 amino acids (inclusive) in length, however, peptides of other lengths, including those over 100 amino acids are also contemplated. In the most preferred embodiments, the peptides are between about 5 and about 10 amino acids (inclusive) in length. A library can have peptides that are homogeneous or heterogeneous in length. By way of non-limiting example, the library can include peptides having a length of 4, 5, 6, 7, 8, 9, or 10; or the combination, or any sub-combination thereof.

In some cases, a binding peptide (e.g., a peptide having a binding sequence where the peptide binds to a skin protein such as keratin) has a length of 5, 6, 7, or 8 amino acids (e.g., see SEQ ID NOs: 301-313, and 326-417), and is considered to be a skin penetrating peptide. Thus, in some cases a skin penetrating peptide can have a length in a range of from 5-8 amino acids (e.g., 5, 6, 7, or 8 amino acids).

In some cases a binding peptide (e.g., a peptide having a binding sequence where the peptide binds to a skin protein such as keratin) is flanked by C residues and is considered to be a skin penetrating peptide. Thus in some cases (e.g., if the C residues are the only amino acids flanking the binding sequence), a subject skin penetrating peptide can have a length in a range of from 7 to 10 amino acids (e.g., 7, 8, 9, or 10 amino acids) (e.g., see SEQ ID NOs: 151-163, and 176-267).

In some cases in which the binding sequence is flanked by C residues, additional amino acids are also present (e.g., in some cases a binding sequence can be flanked by two amino acids on each side, e.g., AC on the N-terminal side and CG on the C-terminal side). In some cases, a subject skin penetrating peptide can have a length in a range of from 9 to 12 amino acids (e.g., 9, 10, 11, or 12 amino acids) (e.g., see SEQ ID NOs: 1-13, and 26-117). As such, in some cases, a subject skin penetrating peptide has a length in a range of from 5 to 12 amino acids (e.g., 7 to 12 or 9 to 12 amino acids). In some cases, a subject skin penetrating peptide has a length of 9 amino acids. In some cases, a subject skin penetrating peptide has a length of 10 amino acids. In some cases, a subject skin penetrating peptide has a length of 11 amino acids. In some cases, a subject skin penetrating peptide has a length of 12 amino acids. In some cases, one or more (e.g., 2 or more, 3 or more, 4 or more, 5 or more, 6 or more, etc.) additional amino acids are positioned internal to the flanking C residues (e.g., C—XX-binding peptide-XX—C). In some cases, one or more (e.g., 2 or more, 3 or more, 4 or more, 5 or more, 6 or more, etc.) additional amino acids are positioned external to the flanking C residues (e.g., XX—C-binding peptide-C—XX). In some cases, one or more (e.g., 2 or more, 3 or more, 4 or more, 5 or more, 6 or more, etc.) additional amino acids are positioned internal and external to the flanking C residues (e.g., XX—C—XX-binding peptide-XX—C—XX).

In some cases, a subject skin penetrating peptide has a length in a range of from 3 to 100 amino acids (e.g., 4 to 100, 5 to 100, 7 to 100, 9 to 100, 11 to 100, 12 to 100, 4 to 30, 5 to 30, 7 to 30, 9 to 30, 11 to 30, 12 to 30, 4 to 20, 5 to 20, 7 to 20, 9 to 20, 11 to 20, or 12 to 20 amino acids). In some cases, a subject skin penetrating peptide has a length in a range of from 5 to 100 amino acids. In some cases, a subject skin penetrating peptide has a length in a range of from 7 to 100 amino acids. In some cases, a subject skin penetrating peptide has a length in a range of from 9 to 100 amino acids. In some cases, a subject skin penetrating peptide has a length in a range of from 11 to 100 amino acids. In some cases, a subject skin penetrating peptide has a length in a range of from 3 to 30 amino acids. In some cases, a subject skin penetrating peptide has a length in a range of from 5 to 30 amino acids. In some cases, a subject skin penetrating peptide has a length in a range of from 7 to 30 amino acids. In some cases, a subject skin penetrating peptide has a length in a range of from 9 to 30 amino acids. In some cases, a subject skin penetrating peptide has a length in a range of from 11 to 30 amino acids.

In some embodiments, the peptide can be a naturally occurring protein or a fragment thereof. In some embodiments, the peptide sequence is at least 80%, 90%, 95%, or even 100% identical to a fragment of a naturally occurring protein, but is not the full length of the naturally occurring protein. The peptide can be, for example 1, 2, 3, 4, 5, 10, 15, 20, 25, 50, 100, or more amino acid shorter in length than the naturally occurring protein. In some embodiments, the peptide has different properties compared to the full length protein. Preferably the peptide has (1) increased binding to a skin protein; (2) increased binding to an active agent; (3) increased absorption or penetration of the active into or through the skin; (4) reduced toxicity; or a combination thereof compared to the naturally occurring protein.

In some embodiments, the peptide is a cyclic peptide. Cyclic peptides are polypeptide chains wherein the amino termini and carboxyl termini, amino termini and side chain, carboxyl termini and side chain, or side chain and side chain are linked with a bond, preferably a covalent bond, which generates a ring.

In particularly preferred embodiments, the cyclic peptide is formed by a disulfide bond between to cysteines. For example, the peptide can include a cysteine in the N-terminal half of the peptide and a cysteine in the C-terminal half of the peptide. In some embodiments, the peptide includes only two cysteines. In particular embodiments, one cysteine is in N-terminal half of the peptide and one is in the C-terminal half of the peptide. In some embodiments, two cysteines are present in the same half of the peptide (e.g. the N-terminal half or C-terminal half). Preferably, the cysteines are positioned such that the resulting peptides form cyclic peptides via a disulfide bond under physiological conditions. The cysteines can be equidistant from the N-terminus and C-terminus. For example, the cysteines can be the N-terminal and C-terminal residues, or one residue each from N-terminus and the C-terminus, or two residues each from N-terminus and the C-terminus, or three residues each from N-terminus and the C-terminus, or four residues each from N-terminus and the C-terminus, or five residues each from N-terminus and the C-terminus, or six residues each from N-terminus and the C-terminus, or seven residues each from N-terminus and the C-terminus, or eight residues each from N-terminus and the C-terminus, or nine residues each from N-terminus and the C-terminus, or ten residues each from N-terminus and the C-terminus, etc. In some embodiments, the cysteines are not equidistant from the N- and C-termini. In some embodiments, the N-terminal residues are Ala-Cys, the C-terminal residues are Cys-Gly. In some embodiments, the cysteines have an integer between about 1 and 98 about amino acids, inclusive, between them.

In some embodiments, a subject polypeptide is modified, e.g., using ordinary molecular biological techniques and/or synthetic chemistry, e.g., to improve stability/resistance to proteolytic degradation, to optimize solubility properties, to enhance protein activity, to render it more suitable as a therapeutic agent, and the like. Analogs of subject polypeptides can be used, including those containing residues other than naturally occurring L-amino acids, e.g. D-amino acids or non-naturally occurring synthetic amino acids. D-amino acids may be substituted for some or all of the amino acid residues.

A subject polypeptide can be prepared by in vitro synthesis using any convenient method, e.g., using conventional methods known in the art. Various commercial synthetic apparatuses are available, for example, automated synthesizers. By using synthesizers, naturally occurring amino acids may be substituted with unnatural amino acids. The particular sequence and the manner of preparation can be determined by convenience, economics, purity required, and the like.

If desired, various groups may be introduced into a subject polypeptide (e.g., during synthesis, or during expression, after synthesis, etc.), which allow for linking to other molecules or to a surface. For example, cysteines can be used to make thioethers, histidines for linking to a metal ion complex, carboxyl groups for forming amides or esters, amino groups for forming amides, and the like.

In some cases, a subject polypeptide can be isolated and purified in accordance with a convenient method of recombinant synthesis. For example, a subject polypeptide can be produced in a cell such as a prokaryotic cell. A lysate may be prepared of the expression host and the lysate purified, e.g., using HPLC, exclusion chromatography, gel electrophoresis, affinity chromatography, or other purification technique. A polypeptide can be at least 20% by weight of the desired product, more usually at least about 75% by weight, preferably at least about 95% by weight, and for therapeutic purposes, usually at least about 99.5% by weight, in relation to contaminants related to the method of preparation of the product and its purification. In some cases, the percentages will be based upon total protein.

Preferably, the peptide binds, preferably binds specifically, to one or more skin proteins. Skin proteins include, but are not limited to, proteins expressed by, elevated in, or secreted or extruded by a tissue (e.g., epidermis, dermis, hypodermis, or stratum germinativum, stratum spinosum, stratum granulosum, stratum lucidum, stratum corneum, stratum basale, or stratum spinosum) or cell type (e.g., melanocytes, Langerhans cells, and keratinocytes) of the skin. In preferred embodiments, the protein is selected from the group consisting of keratin, collagen, plectin, actin, or tubulin, and is most preferably a keratin. Keratin is a family of fibrous structural proteins whose monomers form intermediate filaments. Keratin proteins are well known in the art and can be paired in different combinations to form intermediate filaments with different properties. Exemplary pairs include, but are not limited to,

| A (neutral-basic) | B (acidic) | Prominent Tissue/Cell Type |
|---|---|---|
| keratin 1, keratin 2 | keratin 9, keratin 10 | stratum corneum, |
| | | keratinocytes |
| keratin 4 | keratin 13 | stratified epithelium |
| keratin 5 | keratin 14, keratin 15 | stratified epithelium |
| keratin 6 | keratin 16, keratin 17 | squamous epithelium |
| keratin 7 | keratin 19 | ductal epithelia |
| keratin 3 | keratin 12 | cornea |

In some embodiments, the skin protein is a keratin that is prominent in stratified epithelium. For example, in particular embodiments the skin protein is keratin 5, keratin 14, or a combination thereof.

In some embodiments, the peptide interacts or binds to solvent accessible regions or residues of the skin protein, the active agent or a combination thereof. The solvent accessible regions or residues can be defined as those that exhibit a relative solvent accessibility higher than 30%, 40%, 50%, 60%, 70%, 80%, or 90%, as defined by the program NACCESS.

In preferred embodiments, the peptide binds to a skin protein (e.g., keratin) with K_{d} of at least about 10⁻³ M. However, it is believed that some peptides may bind too tightly to a skin protein, hindering the ability of the peptide to deliver an active agent into or through the skin. Therefore, in some embodiments, the peptide binds to a skin protein (e.g., keratin) with K_{d} not greater than about 10⁻⁸M. In preferred embodiments, the peptide binds to a skin protein, preferably a keratin, particularly a keratin5/keratin14 intermediate filament, with a K_{d} of between about 10⁻³ M and about 10⁻⁸M, inclusive. For example, the K_{d} of between about 10⁴ M and about 10⁻⁸M, about 10⁻⁵ M and about 10⁻⁸M, about 10⁻⁶ M and about 10⁻⁸M, about 10⁻⁷ M and about 10⁻⁸M, about 10⁻³ M and about 10⁻⁷ M, 10⁴ M and about 10⁻⁷ M, about 10⁻⁵ M and about 10⁻⁷ M, about 10⁻⁶ M and about 10⁻⁷ M, about 10⁻³ M and about 10⁻⁶ M, about 10⁴ M and about 10⁻⁶ M, about 10⁻⁵ M and about 10⁻⁶ M, about 10⁻³ M and about 10⁻⁵ M, about 10⁴ M and about 10⁻⁵ M, about 10⁻³ M and about 10⁴ M, about 10⁻⁵ M and about 10⁻⁶ M, about 10⁻⁶ M and about 10⁻⁷ M, or about 10⁻⁷ M and about 10⁻⁸M. In some embodiments, the peptide binds to a skin protein, preferably a keratin, particularly a keratin5/keratin14 intermediate filament, with a K_{d} of about 10⁻⁸M, 10⁻⁷ M, 10⁻⁶ M, 10⁻⁵ M, 10⁴ M, or 10⁻³ M.

The K_{d} can be measured using any suitable means. Examples include the methods of measuring molecule-molecule (e.g., protein-protein) interactions discussed below. In a particular embodiment, the K_{d} is determined using affinity chromatography.

Typically, the peptide increases absorption or penetration of an effective amount of an active agent into one or more tissues (e.g., epidermis, dermis, hypodermis, or stratum germinativum, stratum spinosum, stratum granulosum, stratum lucidum, stratum corneum, stratum basale, or stratum spinosum) or cells (e.g., melanocytes, Langerhans cells, and keratinocytes) of the skin. The penetration or absorption can be intracellular, extracellular, transcellular, or a combination thereof. For example, in some embodiments, the peptide facilitates transport of the active agent across the cell membrane (e.g., a cell penetrating peptide). The peptide and/or active agent can be retained within the cell (intracellular), or pass completely through the cell and into an underlying extracellular or adjacent intracellular space (transcellular). The peptide can also facilitate transportation of the active agent through one or more layers of skin without crossing the cell membrane (extracellular). In many embodiments, the transdermal delivery includes a combination of two or more of intracellular, transcellular, and extracellular transport.

Among the various skin layers, the stratum corneum (SC) is particularly important in protecting underlying organs from foreign agents, such as pathogens and toxins. The SC includes keratin-rich cells embedded in multiple lipid bilayers, and the hydrophobicity and the densely packed structure of this layer makes permeation of even small therapeutically active ingredients difficult. In particularly preferred embodiments, the peptide increases absorption or penetration of active agent into or through the stratum corneum.

In some embodiments, the site of treatment is one or more tissues or cells of the skin, and the peptide need not facilitate delivery of the active agent across the skin and into systemic circulation. In such embodiments, the peptide may facilitate entry into a tissue or cell of the skin where it is retained. This is particularly beneficial when the subject is need of treatment for a skin or skin-related condition, disease, or disorder. In some embodiments, the peptide facilitates traversal of the active agent across the skin to an underlying tissue or into systemic circulation.

2. Preferred Peptide Motifs

In some embodiments, the peptide includes or consists of the amino acid sequence and structure:

```
(SEQ ID NO: 16)
Cys - X1 - Xn - Cys
    |  |
        |_____S-S____|
or
(SEQ ID NO: 17)
Ala - Cys - X1 - Xn - Cys - Gly
                |  |
                    |_____S-S____|
```

wherein each “X” is independently any amino acid, or a subset thereof, for example the 19 canonical amino acids excluding cysteine; wherein “n” is 0 or an integer between 1 and 100 inclusive; and wherein peptide cyclization is achieved through the formation of a disulfide bond between two cysteines, for example the two cysteines framing the sequence (e.g., “Cys” of SEQ ID NO:16 or 17, above). Preferably n is 0, 1, 2, 3, 4, 5, 6, 7, 8, 9, or 10. In particular embodiments, the peptide is a pentamer (n=5), hexamer (n=6), heptamer (n=7), or octamer (n=8).

In some embodiments, the peptide includes one or more of the following amino acid sequence motifs: NHN, QHN, NRN, and QRQ (N: Asparagine, Q: Glutamine, H: Histidine, R: Arginine). In preferred embodiments, the peptide includes one, two, three or more hydrogen bonding amino acids; one, two, three or more Glycines; one, two, three or more Alanines; or a combination thereof.

The disclosed peptides include those having the amino acid sequences provided, as well as peptides having one or more amino acid substitutions, e.g., one or more conservative amino acid substitutions, relative to the sequences provided, wherein the peptides retains the capability of penetrating the skin or a cell thereof.

3. Preferred Peptide Sequences

In some more specific embodiments, each “X” is selected from a more restrictive subset of amino acids. For example, the peptide can be a disulphide-cyclic peptide including or consisting of the amino acid sequence: cyclo[C-X1-X2-X3-X4-X5-C] (SEQ ID NO:18), wherein
“C” is a Cysteine;
“X1” is Serine, Threonine, Asparagine, Glutamine, or Glycine;
“X2” is Histidine, Asparagine, or Glutamine;
“X3” is Histidine, Arginine, Asparagine, or Glutamine;
“X4” is Histidine, Asparagine, Glutamine, Serine, or Threonine;
“X5” is Serine, Threonine, Glycine, or Alanine; and
wherein peptide cyclization is achieved through the formation of a disulfide bond between two cysteines, for example the two cysteines framing the sequence (e.g., “C” of SEQ ID NO:18).

The peptide can be a disulphide-cyclic peptide including or consisting of the amino acid sequence: cyclo[C—X1-X2-X3-X4-X5-X6-C] (SEQ ID NO:19), wherein
“C” is Cysteine;
“X1” is Serine, Threonine, Asparagine, Glutamine, or Glycine;
“X2” is Serine, Threonine, Asparagine, or Glutamine;
“X3” is Histidine, Arginine, Lysine, Asparagine, Glutamine, Glycine, or Alanine;
“X4” is Serine, Threonine, Asparagine, Glutamine, Glycine, or Arginine;
“X5” is Histidine, Arginine, Lysine, Asparagine, Glutamine, Serine, or Threonine;
“X6” is Asparagine, Glutamine, Serine, Threonine, Arginine, Glycine, or Alanine; and
wherein peptide cyclization is achieved through the formation of a disulfide bond between two cysteines, for example the two cysteines framing the sequence (e.g., “C” of SEQ ID NO:19).

In some embodiments, the peptide is a disulphide-cyclic peptide including the amino acid sequence: cyclo[C—X1-X2-X3-X4-X5-X6-X7-C] (SEQ ID NO:20), wherein
“C” is a Cysteine;
“X1” is Serine, Threonine, Glycine, Alanine, or Valine;
“X2” is Glycine, Alanine, Valine, Leucine, Serine, or Threonine;
“X3” is Glycine, Alanine, Serine, or Threonine;
“X4” is Asparagine, Glutamine, Arginine, or Lysine;
“X5” is Histidine, Asparagine, Glutamine, Tryptophan, Serine, or Threonine;
“X6” is Serine, Threonine, Histidine, Asparagine, Glutamine, Glycine, or Alanine;
“X7” is Serine, Threonine, Histidine, Asparagine, Glutamine, Glycine, or Alanine; and
wherein peptide cyclization is achieved through the formation of a disulfide bond between two cysteines, for example the two cysteines framing the sequence (e.g., “C” of SEQ ID NO:20).

The peptide can be a disulphide-cyclic peptide including or consisting of the amino acid sequence cyclo[C—X1-X2-X3-X4-X5-X6-X7-X8-C] (SEQ ID NO:21), wherein
“C” is a Cysteine;
“X1” is Serine, Threonine, Asparagine, Glutamine, Glycine, or Alanine;
“X2” is Alanine, Serine, Threonine, or Arginine;
“X3” is Histidine, Asparagine, Glutamine, Lysine, or Arginine;
“X4” is Asparagine, Arginine, Histidine, or Tryptophan;
“X5” is Glycine, Alanine, Arginine, Glutamine, Lysine, or Arginine;
“X6” is Histidine, Tryptophan, Glycine, or Alanine;
“X7” is Serine, Threonine, Asparagine, or Glutamine;
“X8” is Serine, Threonine, Glycine, or Alanine; and
wherein peptide cyclization is achieved through the formation of a disulfide bond between two cysteines, for example the two cysteines framing the sequence (e.g., “C” of SEQ ID NO:21).

4. Exemplary Keratin-Binding Peptides

Exemplary keratin-binding peptides are provided. The peptides can, for example, include or consist of any of the amino acid sequences recited in Table 1:

| | PEPTIDE | SEQ ID |
|---|---|---|
| # | SEQUENCE | NO: |
| 1 | ACSATLQHSCG | 4 |
| 2 | ACTIQHRAECG | 26 |
| 3 | ACTIQHGRSCG | 27 |
| 4 | ACVSAGQNHCG | 28 |
| 5 | ACVSIGNHNCG | 29 |
| 6 | ACVSANHQICG | 30 |
| 7 | ACVSATGNHCG | 31 |
| 8 | ACAEGINVHCG | 32 |
| 9 | ACVSEHINGCG | 33 |
| 10 | ACTIQHRAFCG | 34 |
| 11 | ACSLTVNWNCG | 6 |
| 12 | ACRVAHFITCG | 35 |
| 13 | ACVSEQHNICG | 36 |
| 14 | ACVSANHQTCG | 37 |
| 15 | ACTSVINEHCG | 38 |
| 16 | ACAEHRSQTCG | 39 |
| 17 | ACRVTNHQSCG | 40 |
| 18 | ACTIHNRQSCG | 41 |
| 19 | ACVSENHQGCG | 42 |
| 20 | ACVSAGNHQCG | 43 |
| 21 | ACLSVNHNACG | 7 |
| 22 | ACRVAIHGNCG | 44 |
| 23 | ACVSATQHNCG | 45 |
| 24 | ACVSATFNHCG | 46 |
| 25 | ACTHNRQSFCG | 47 |
| 26 | ACIEVNHNRCG | 48 |
| 27 | ACVSEFNTHCG | 49 |
| 28 | ACHISGVFNCG | 50 |
| 29 | ACIEVNHNSCG | 51 |
| 30 | ACHISGEARCG | 52 |
| 31 | ACIVNHFRQCG | 53 |
| 32 | ACHSAGIFVCG | 54 |
| 33 | ACIEVNHQSCG | 55 |
| 34 | ACVIHFTRNCG | 56 |
| 35 | ACGVQHSRNCG | 57 |
| 36 | ACVSNQIERCG | 58 |
| 37 | ACVSTGNHNCG | 59 |
| 38 | ACTHENRQSCG | 60 |
| 39 | ACVSAFTHGCG | 61 |
| 40 | ACIEANTFHCG | 62 |
| 41 | ACAEQGHTRCG | 63 |
| 42 | ACTQVNHRSCG | 64 |
| 43 | ACAVRENQTCG | 65 |
| 44 | ACTIANRQSCG | 66 |
| 45 | ACTIVNHRSCG | 67 |
| 46 | ACAEIVGHRCG | 68 |
| 47 | ACRVNHTSICG | 69 |
| 48 | ACAEIHTGRCG | 70 |
| 49 | ACRVNHTSACG | 71 |
| 50 | ACRVNHASQCG | 72 |
| 51 | ACVSNAQERCG | 73 |
| 52 | ACQVTEIANCG | 74 |
| 53 | ACHTNAVSQCG | 75 |
| 54 | ACQVNHFISCG | 76 |
| 55 | ACQVNFASTCG | 77 |
| 56 | ACHVQGENSCG | 78 |
| 57 | ACQRHVASTCG | 79 |
| 58 | ACTSRNQHVCG | 80 |
| 59 | ACTGSTQHQCG | 1 |
| 60 | ACRVSFHTQCG | 81 |
| 61 | ACQRITSHACG | 82 |
| 62 | ACRAHFGESCG | 83 |
| 63 | ACAEIGRNSCG | 84 |
| 64 | ACTINHRVSCG | 85 |
| 65 | ACQVFATSHCG | 86 |
| 66 | ACSRVNTGQCG | 87 |
| 67 | ACTINHRSVCG | 88 |
| 68 | ACTIHSVQNCG | 89 |
| 69 | ACQVTAGRSCG | 90 |
| 70 | ACTNAIRFSCG | 91 |
| 71 | ACQVAGIHNCG | 92 |
| 72 | ACTIEGFANCG | 93 |
| 73 | ACTNFEGSRCG | 94 |
| 74 | ACQVNRASHCG | 95 |
| 75 | ACIVQANERCG | 96 |
| 76 | ACVFSNQITCG | 97 |
| 77 | ACVFSITGQCG | 98 |
| 78 | ACVSHTNRFCG | 99 |
| 79 | ACVSGFETACG | 100 |
| 80 | ACRVAQTGICG | 101 |
| 81 | ACVIRQSNTCG | 102 |
| 82 | ACVSETRNIC | 103 |
| 83 | ACVNARISFC | 104 |
| 84 | ACAEIFGQNCG | 105 |
| 85 | ACAERSGIVCG | 106 |
| 86 | ACAEHNISQCG | 107 |
| 87 | ACNGTGSHQCG | 108 |
| 88 | ACVSFINTQCG | 109 |
| 89 | ACSRQHNEFCG | 110 |
| 90 | ACQNFIERACG | 111 |
| 91 | ACVSFGIENCG | 112 |
| 92 | ACSASQVHNCG | 9 |
| 93 | ACVNTERFGCG | 113 |
| 94 | ACNSTAVQGCG | 114 |
| 95 | ACNSITERVCG | 115 |
| 96 | ACVSNEFGTCG | 116 |
| 97 | ACNGTGSHQCG | 10 |
| 98 | ACAEIQGNRCG | 117 |
| 99 | ACSASTNHNCG | 8 |
| 100 | ACSVTTQHQCG | 11 |

The peptides were identified in the in silico screen described in the working Examples below, and prior to a second round of screening for binding to CSA.

The amino acid sequences of the best keratin-binding pentamer, hexamer, heptamer, and octamer disulfide-bonded, cyclic peptides selected through the in silico exemplified below are set forth in Table 2:

| Library | Sequence | SEQ ID NO: | K_{D} (M) |
|---|---|---|---|
| Pentamer (5) | ACSHNHTCG | 2 | 5.21 × 10⁻⁴ |
| Hexamer (6) | ACTHTGRNCG | 3 | 1.02 × 10⁻⁴ |
| Heptamer (7) | ACSATLQHSCG | 4 | 6.79 × 10⁻⁵ |
| Octamer (8) | ACNAHQARSTCG | 5 | 9.34 × 10⁻⁶ |

In some embodiments, the skin penetrating peptide includes or consists of any of the amino acid sequences set forth in Table 2.

5. Exemplary Cyclosporine A-Binding Peptides

The peptide can bind to both a skin protein such as a keratin and an active agent. In the most preferred embodiments, the peptide improves absorption, penetration, or traversal of the active agent into or through the skin. In some embodiments, the peptide improves systemic circulation of the active agent following application of a combination of the peptide and active agent to the skin (e.g., dermal or transdermal delivery).

| ID | Sequence | SEQ ID NO: |
|---|---|---|
| 1 (SP7-1) | ACSATLQHSCG | 4 |
| 5 (SP7-2) | ACSLTVNWNCG | 6 |
| 10 (SP7-3) | ACLSVNHNACG | 7 |
| 17 (SPACE™) | ACTGSTQHQCG | 1 |
| 26 (SP7-5) | ACSASTNHNCG | 8 |
| 30 | ACSASQVHNCG | 9 |
| 40 | ACNGTGSHQCG | 10 |
| 50 | ACSVTTQHQCG | 11 |
| 75 | ACVSVTNHQCG | 12 |
| 100 (SP7-4) | ACTSTGRNACG | 13 |

In some embodiments, the skin penetrating peptide includes or consists of any of the amino acid sequences set forth in Table 3.

B. Active Agents

The disclosed skin penetrating peptides can be used to facilitate skin penetration of one or more active agents. Typically, the active agent is a pharmaceutical or therapeutic active agent. The peptides can be bound, associated, attached, linked, or conjugated to the active agent. In some embodiments, the peptide is not bound, associated, attached, linked, or conjugated to the active agent. In some embodiments, the peptide is covalently conjugated, optionally via a linker, to the active agent.

General classes of active agents which may be delivered include, for example, proteins, peptides, nucleic acids, nucleotides, nucleosides and analogues thereof; as well as pharmaceutical compounds, e.g., low molecular weight compounds, small molecules, etc. In some embodiments the active agent is a small molecule or low molecular weight compound, e.g., a molecule or compound having a molecular weight of less than or equal to about 1000 Daltons, e.g., less than or equal to about 800 Daltons.

Active agents which can be delivered using the penetrating peptides disclosed herein include agents which act on the peripheral nerves, adrenergic receptors, cholinergic receptors, the skeletal muscles, the cardiovascular system, smooth muscles, the blood circulatory system, synaptic sites, neuroeffector junction sites, endocrine and hormone systems, the immunological system, the reproductive system, the skeletal system, autacoid systems, the alimentary and excretory systems, the histamine system and the central nervous system.

Suitable active agents may be selected, for example, from dermatological agents, anti-neoplastic agents, cardiovascular agents, renal agents, gastrointestinal agents, rheumatologic agents, immunological agents, and neurological agents among others.

In some embodiments, the active agent is a label. Suitable labels include, e.g, radioactive isotopes, fluorescers, chemiluminescers, chromophores, enzymes, enzyme substrates, enzyme cofactors, enzyme inhibitors, chromophores, dyes, metal ions, magnetic particles, nanoparticles and quantum dots.

The active agent may be present in any suitable concentration in the compositions disclosed herein. Suitable concentrations may vary depending on the potency of the active agent, active agent half-life, etc. In addition, penetrating peptide compositions according to the present disclosure may include one or more active agents, e.g., a combination of two or more of the active agents described above. Preferably, the active agent is present in an effective amount sufficient to alleviate one or more symptoms of a disorder, disease, or condition being treated, or to otherwise provide a desired pharmacologic and/or physiologic effect when administered in combination with a skin penetrating peptide.

In a particularly preferred embodiment, the active agent is CsA, an immunosuppressant drug used to treat a number of disease including to prevent graft verse host disease and organ rejection, rheumatoid arthritis and related diseases, psoriasis, dry eyes, nummular keratitis particularly following adenoviral keratoconjunctivitis, atopic dermatitis, Kimura disease, pyoderma gangrenosum, chronic autoimmune urticaria, acute systemic mastocytosis, acute severe ulcerative colitis, posterior and intermediate uveitis. In preferred embodiments, CsA is administered to a subject in need thereof in combination with a skin penetrating peptide in an effective amount to reduce the activity of the immune system by interfering with the activity and/or growth of T cells, to reduce or prevent one or more symptoms of a disease or disorder, or a combination thereof.

Additional exemplary active agents are discussed in more detail below.

1. Exemplary Active Agents

a. Dermatological Active Agents

Suitable dermatological agents may include, for example, local anesthetics, anti-inflammatory agents, anti-infective agents, agents to treat acne, anti-virals, anti-fungals, agents for psoriasis such as topical corticosteroids among others.

In some embodiments, the suitable dermatological agent is selected from the following list: 16-17A-Epoxyprogesterone (CAS Registry Number: 1097-51-4), P-methoxycinnamic acid/4-Methoxycinnamic acid (CAS Registry Number: 830-09-1), Octyl Methoxycinnamate (CAS Registry Number: 5466-77-3), Octyl Methoxycinnamate (CAS Registry Number: 5466-77-3), Methyl p-methoxycinnamate (CAS Registry Number: 832-01-9), 4-ESTREN-17.beta.-OL-3-ONE (CAS Registry Number: 62-90-8), Ethyl-p-anisoyl acetate (CAS Registry Number: 2881-83-6), Dihydrouracil (CAS Registry Number: 1904-98-9), Lopinavir (CAS Registry Number: 192725-17-0), RITANSERIN(CAS Registry Number: 87051-43-2), Nilotinib (CAS Registry Number: 641571-10-0); Rocuronium bromide (CAS Registry Number: 119302-91-9), p-Nitrobenzyl-6-(1-hydroxyethyl)-1-azabicyclo(3.2.0)heptane-3,7-dione-2-c- arboxylate (CAS Registry Number: 74288-40-7), Abamectin (CAS Registry Number: 71751-41-2), Paliperidone (CAS Registry Number: 144598-75-4), Gemifioxacin (CAS Registry Number: 175463-14-6), Valrubicin (CAS Registry Number: 56124-62-0), Mizoribine (CAS Registry Number: 50924-49-7), Solifenacin succinate (CAS Registry Number: 242478-38-2), Lapatinib (CAS Registry Number: 231277-92-2), Dydrogesterone (CAS Registry Number: 152-62-5), 2,2-Dichloro-N-[(1R,2S)-3-fluoro-l-hydroxy-1-(4-methylsulfonylphenyl)prop-an-2-yl]acetamide (CAS Registry Number: 73231-34-2), Tilmicosin (CAS Registry Number: 108050-54-0), Efavirenz (CAS Registry Number: 154598-52-4), Pirarubicin (CAS Registry Number: 72496-41-4), Nateglinide (CAS Registry Number: 105816-04-4), Epirubicin (CAS Registry Number: 56420-45-2), Entecavir (CAS Registry Number: 142217-69-4), Etoricoxib (CAS Registry Number: 202409-33-4), Cilnidipine (CAS Registry Number: 132203-70-4), Doxorubicin hydrochloride (CAS Registry Number: 25316-40-9), Escitalopram (CAS Registry Number: 128196-01-0), Sitagliptin phosphate monohydrate (CAS Registry Number: 654671-77-9), Acitretin (CAS Registry Number: 55079-83-9), Rizatriptan benzoate (CAS Registry Number: 145202-66-0), Doripenem (CAS Registry Number: 148016-81-3), Atracurium besylate (CAS Registry Number: 64228-81-5), Nilutamide (CAS Registry Number: 63612-50-0), 3,4-Dihydroxyphenylethanol (CAS Registry Number: 10597-60-1), KETANSERIN TARTRATE (CAS Registry Number: 83846-83-7), Ozagrel (CAS Registry Number: 82571-53-7), Eprosartan mesylate (CAS Registry Number: 144143-96-4), Ranitidine hydrochloride (CAS Registry Number: 66357-35-5), 6,7-Dihydro-6-mercapto-5H-pyrazolo[1,2-a][1,2,4]triazolium chloride (CAS Registry Number: 153851-71-9), Sulfapyridine (CAS Registry Number: 144-83-2), Teicoplanin (CAS Registry Number: 61036-62-2), Tacrolimus (CAS Registry Number: 104987-11-3), LUMIRACOXIB (CAS Registry Number: 220991-20-8), Allyl alcohol (CAS Registry Number: 107-18-6), Protected meropenem (CAS Registry Number: 96036-02-1), Nelarabine (CAS Registry Number: 121032-29-9), Pimecrolimus (CAS Registry Number: 137071-32-0), 4-[6-Methoxy-7-(3-piperidin-l-ylpropoxy)quinazolin-4-yl]-N-(4-propan-2-yl- oxyphenyl)piperazine-l-carboxamide (CAS Registry Number: 387867-13-2), Ritonavir (CAS Registry Number: 155213-67-5), Adapalene (CAS Registry Number: 106685-40-9), Aprepitant (CAS Registry Number: 170729-80-3), Eplerenone (CAS Registry Number: 107724-20-9), Rasagiline mesylate (CAS Registry Number: 161735-79-1), Miltefosine (CAS Registry Number: 58066-85-6), Raltegravir potassium (CAS Registry Number: 871038-72-1), Dasatinib monohydrate (CAS Registry Number: 863127-77-9), OXOMEMAZINE (CAS Registry Number: 3689-50-7), Pramipexole (CAS Registry Number: 104632-26-0), PARECOXIB SODIUM (CAS Registry Number: 198470-85-8), Tigecycline (CAS Registry Number: 220620-09-7), Toltrazuril (CAS Registry Number: 69004-03-1), Vinflunine (CAS Registry Number: 162652-95-1), Drospirenone (CAS Registry Number: 67392-87-4), Daptomycin (CAS Registry Number: 103060-53-3), Montelukast sodium (CAS Registry Number: 151767-02-1), Brinzolamide (CAS Registry Number: 138890-62-7), Maraviroc (CAS Registry Number: 376348-65-1), Doxercalciferol (CAS Registry Number: 54573-75-0), Oxolinic acid (CAS Registry Number: 14698-29-4), Daunorubicin hydrochloride (CAS Registry Number: 23541-50-6), Nizatidine (CAS Registry Number: 76963-41-2), Idarubicin (CAS Registry Number: 58957-92-9), FLUOXETINE HYDROCHLORIDE (CAS Registry Number: 59333-67-4), Ascomycin (CAS Registry Number: 11011-38-4), beta-Methyl vinyl phosphate (MAP) (CAS Registry Number: 90776-59-3), Amorolfine (CAS Registry Number: 67467-83-8), Fexofenadine HCl (CAS Registry Number: 83799-24-0), Ketoconazole (CAS Registry Number: 65277-42-1), 9,10-difluoro-2,3-dihydro-3-me-7-oxo-7H-pyrido-1 (CAS Registry Number: 82419-35-0), Ketoconazole (CAS Registry Number: 65277-42-1), Terbinafine HC1 (CAS Registry Number: 78628-80-5), Amorolfine (CAS Registry Number: 78613-35-1), Methoxsalen (CAS Registry Number: 298-81-7), Olopatadine HC1 (CAS Registry Number: 113806-05-6), Zinc Pyrithione (CAS Registry Number: 13463-41-7), Olopatadine HCl (CAS Registry Number: 140462-76-6), Cyclosporine (CAS Registry Number: 59865-13-3), and Botulinum toxin and its analogs and vaccine components.

b. Protein Active Agents

In some embodiments, the active agent is a protein or peptide. Example of protein active agents include, but are not limited to, cytokines and their receptors, as well as chimeric proteins including cytokines or their receptors, including, for example tumor necrosis factor alpha and beta, their receptors and their derivatives; renin; growth hormones, including human growth hormone, bovine growth hormone, methione-human growth hormone, des-phenylalanine human growth hormone, and porcine growth hormone; growth hormone releasing factor (GRF); parathyroid and pituitary hormones; thyroid stimulating hormone; human pancreas hormone releasing factor; lipoproteins; colchicine; prolactin; corticotrophin; thyrotropic hormone; oxytocin; vasopressin; somatostatin; lypressin; pancreozymin; leuprolide; alpha-1-antitrypsin; insulin A-chain; insulin B-chain; proinsulin; follicle stimulating hormone; calcitonin; luteinizing hormone; luteinizing hormone releasing hormone (LHRH); LHRH agonists and antagonists; glucagon; clotting factors such as factor VIIIC, factor IX, tissue factor, and von Willebrands factor; anti-clotting factors such as Protein C; atrial natriuretic factor; lung surfactant; a plasminogen activator other than a tissue-type plasminogen activator (t-PA), for example a urokinase; bombesin; thrombin; hemopoietic growth factor; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; chorionic gonadotropin; gonadotropin releasing hormone; bovine somatotropin; porcine somatotropin; a microbial protein, such as beta-lactamase; DNase; inhibin; activin; vascular endothelial growth factor (VEGF); receptors for hormones or growth factors; integrin; protein A or D; rheumatoid factors; a neurotrophic factor such as bone-derived neurotrophic factor (BDNF), neurotrophin-3, 4, -5, or -6 (NT-3, NT-4, NT-5, or NT-6), or a nerve growth factor such as NGF-.beta.; platelet-derived growth factor (PDGF); fibroblast growth factor such as acidic FGF and basic FGF; epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-alpha (e.g., interferon.alpha.2A), -beta, -gamma, -lambda and consensus interferon; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the HIV-1 envelope glycoprotein, gp120, gp160 or fragments thereof; transport proteins; homing receptors; addressins; fertility inhibitors such as the prostaglandins; fertility promoters; regulatory proteins; antibodies (including fragments thereof) and chimeric proteins, such as immunoadhesins; precursors, derivatives, prodrugs and analogues of these compounds, and pharmaceutically acceptable salts of these compounds, or their precursors, derivatives, prodrugs and analogues.

Suitable proteins or peptides may be native or recombinant and include, e.g., fusion proteins.

In some embodiments, the protein is a growth hormone, such as human growth hormone (hGH), recombinant human growth hormone (rhGH), bovine growth hormone, methione-human growth hormone, des-phenylalanine human growth hormone, and porcine growth hormone; insulin, insulin A-chain, insulin B-chain, and proinsulin; or a growth factor, such as vascular endothelial growth factor (VEGF), nerve growth factor (NGF), platelet-derived growth factor (PDGF), fibroblast growth factor (FGF), epidermal growth factor (EGF), transforming growth factor (TGF), and insulin-like growth factor-I and -II (IGF-I and IGF-II).

In some embodiments, the protein is Glucagon-like peptide-1 (GLP-1) or a precursor, derivative, prodrug, or analogue thereof.

c. Nucleic Acids Active Agents

In some embodiments, the active agent is a nucleic acid. Nucleic acid active agents include nucleic acids as well as precursors, derivatives, prodrugs and analogues thereof, e.g., therapeutic nucleotides, nucleosides and analogues thereof; therapeutic oligonucleotides; and therapeutic polynucleotides. Suitable nucleic acid active agents may include for example ribozymes, antisense oligodeoxynucleotides, aptamers, siRNA, microRNA (miRs) or antagomirs (e.g., anti-miRs) thereof. Examples of suitable nucleoside analogues include, but are not limited to, cytarabine (araCTP), gemcitabine (dFdCTP), and floxuridine (FdUTP). In some embodiments, a suitable nucleic acid active agent is an interfering RNA, e.g., shRNA, miRNA or siRNA. Suitable siRNAs include, for example, IL-7 (Interleukin-7) siRNA, IL-10 (Interleukin-10) siRNA, IL-22 (Interleukin-22) siRNA, IL-23 (Interleukin 23) siRNA, CD86 siRNA, KRT6a (keratin 6A) siRNA, K6a N171K (keratin 6a N171K) siRNA, TNFa (tumor necrosis factor a) siRNA, TNFR1 (tumor necrosis factor receptor-1) siRNA, TACE (tumor necrosis factor (TNF)-a converting enzyme) siRNA, RRM2 (ribonucleotide reductase subunit-2) siRNA, and VEGF (vascular endothelial growth factor) siRNA. mRNA sequences of the human gene targets of these siRNAs are known in the art. In addition a variety of methods and techniques are known in the art for selecting a particular mRNA target sequence during siRNA design. See, e.g., the publicly available siRNA design tool provided by the Whitehead Institute of Biomedical Research at MIT.

d. Other Active Agents

A variety of additional active agent compounds may be used in combination with disclose skin penetrating peptides. Suitable compounds may include compounds directed to one or more of the following drug targets: Kringle domain, Carboxypeptidase, Carboxylic ester hydrolases, Glycosylases, Rhodopsin-like dopamine receptors, Rhodopsin-like adrenoceptors, Rhodopsin-like histamine receptors, Rhodopsin-like serotonin receptors, Rhodopsin-like short peptide receptors, Rhodopsin-like acetylcholine receptors, Rhodopsin-like nucleotide-like receptors, Rhodopsin-like lipid-like ligand receptors, Rhodopsin-like melatonin receptors, Metalloprotease, Transporter ATPase, Carboxylic ester hydrolases, Peroxidase, Lipoxygenase, DOPA decarboxylase, A/G cyclase, Methyltransferases, Sulphonylurea receptors, other transporters (e.g., Dopamine transporter, GABA transporter 1, Norepinephrine transporter, Potassium-transporting ATPase a-chain 1, Sodium-(potassium)-chloride cotransporter 2, Serotonin transporter, Synaptic vesicular amine transporter, and Thiazide-sensitive sodium-chloride cotransporter), Electrochemical nucleoside transporter, Voltage-gated ion channels, GABA receptors (Cys-Loop), Acetylcholine receptors (Cys-Loop), NMDA receptors, 5-HT3 receptors (Cys-Loop), Ligand-gated ion channels Glu: kainite, AMPA Glu receptors, Acid-sensing ion channels aldosterone, Ryanodine receptors, Vitamin K epoxide reductase, MetGluR-like GABA_{B} receptors, Inwardly rectifying K.sup.+channel, NPC1L1, MetGluR-like calcium-sensing receptors, Aldehyde dehydrogenases, Tyrosine 3-hydroxylase, Aldose reductase, Xanthine dehydrogenase, Ribonucleoside reductase, Dihydrofolate reductase, IMP dehydrogenase, Thioredoxin reductase, Dioxygenase, Inositol monophosphatase, Phosphodiesterases, Adenosine deaminase, Peptidylprolyl isomerases, Thymidylate synthase, Aminotransferases, Farnesyl diphosphate synthase, Protein kinases, Carbonic anhydrase, Tubulins, Troponin, Inhibitor of I.kappa.B kinase-.beta., Amine oxidases, Cyclooxygenases, Cytochrome P450s, Thyroxine 5-deiodinase, Steroid dehydrogenase, HMG-CoA reductase, Steroid reductases, Dihydroorotate oxidase, Epoxide hydrolase, Transporter ATPase, Translocator, Glycosyltransferases, Nuclear receptors NR3 receptors, Nuclear receptors: NR1 receptors, and Topoisomerase.

In some embodiments, the active agent is a compound targeting one of rhodopsin-like GPCRs, nuclear receptors, ligand-gated ion channels, voltage-gated ion channels, penicillin-binding protein, myeloperoxidase-like, sodium: neurotransmitter symporter family, type II DNA topoisomerase, fibronectin type III, and cytochrome P450.

In some embodiments, the active agent is an anticancer agent. Suitable anticancer agents include, but are not limited to, Actinomycin D, Alemtuzumab, Allopurinol sodium, Amifostine, Amsacrine, Anastrozole, Ara-CMP, Asparaginase, Azacytadine, Bendamustine, Bevacizumab, Bicalutimide, Bleomycin (e.g., Bleomycin A₂ and B₂), Bortezomib, Busulfan, Camptothecin sodium salt, Capecitabine, Carboplatin, Carmustine, Cetuximab, Chlorambucil, Cisplatin, Cladribine, Clofarabine, Cyclophosphamide, Cytarabine, Dacarbazine, Dactinomycin, Daunorubicin, Daunorubicin liposomal, Dacarbazine, Decitabine, Docetaxel, Doxorubicin, Doxorubicin liposomal, Epirubicin, Estramustine, Etoposide, Etoposide phosphate, Exemestane, Floxuridine, Fludarabine, Fludarabine phosphate, 5-Fluorouracil, Fotemustine, Fulvestrant, Gemcitabine, Goserelin, Hexamethylmelamine, Hydroxyurea, Idarubicin, Ifosfamide, Imatinib, Irinotecan, Ixabepilone, Lapatinib, Letrozole, Leuprolide acetate, Lomustine, Mechlorethamine, Melphalan, 6-Mercaptopurine, Methotrexate, Mithramycin, Mitomycin C, Mitotane, Mitoxantrone, Nimustine, Ofatumumab, Oxaliplatin, Paclitaxel, Panitumumab, Pegaspargase, Pemetrexed, Pentostatin, Pertuzumab, Picoplatin, Pipobroman, Plerixafor, Procarbazine, Raltitrexed, Rituximab, Streptozocin, Temozolomide, Teniposide, 6-Thioguanine, Thiotepa, Topotecan, Trastuzumab, Treosulfan, Triethylenemelamine, Trimetrexate, Uracil Nitrogen Mustard, Valrubicin, Vinblastine, Vincristine, Vindesine, Vinorelbine, and analogues, precursors, derivatives and pro-drugs thereof.

Active agents of interest for use in the disclosed penetrating peptide compositions may also include opioids and derivatives thereof as well as opioid receptor agonists and antagonists, e.g., naltrexone, naloxone, nalbuphine, fentanyl, sufentanil, oxycodone, and pharmaceutically acceptable salts and derivatives thereof.

2. Active Agent Carriers

The disclosed skin penetrating peptides can be administered in combination with an active agent carrier which in turn includes the active agent and/or the skin penetrating peptide attached thereto and/or dispersed therein. Suitable active agent carriers include, for example, liposomes, nanoparticles, microparticles, micelles, microbubbles, and the like. Techniques for incorporating active agents into such carriers are known in the art.

3. Conjugation

As described above, one or more active agents can be conjugated to or associated with a skin penetrating peptide. Additionally, or alternatively, a skin penetrating peptide can be conjugated or associated with an active agent carrier which in turn includes the active agent attached thereto and/or dispersed therein.

Conjugation techniques generally result in the formation of one or more covalent bonds between the penetrating peptide and either the active agent or an active agent carrier while association techniques generally utilize one or more of hydrophobic, electrostatic or van der Walls interactions. A variety of techniques can be used for conjugating or associating a peptide to an active agent an active agent carrier, e.g., liposomes, nanoparticles, or micelle as described herein.

For example, where the active agent is a peptide or polypeptide, the entire composition, including the penetrating peptide, may be synthesized using standard amino acid synthesis techniques. Other methods including standard molecular biology techniques may be used to express and purify the entire polypeptide sequence including the penetrating peptide. Additional methods of conjugating peptides to other peptides or polypeptides include Cu-catalyzed azide/alkyne [3+2] cycloaddition “Click Chemistry” as described by Rostovtsev et al. (2002) Angew. Chem. Int. Ed. 41: 2596-2599 and Tornoe et al. (2002) J. Org. Chem. 67: 3057-3064; azide/DIFO (Difluorinated Cyclooctyne) Cu-free Click Chemistry as described by Baskin et al. (2007) PNAS Vol. 104, No. 43: 167393-16797; azide/phosphine “Staudinger Reaction” as described by Lin et al. (2005) J. Am. Chem. Soc. 127: 2686-2695; azide/triarylphosphine “Modified Staudinger Reaction” as described by Saxon and Bertozzi (2000) Mar. 17 Science 287(5460):2007-10; and catalyzed olefin cross metathesis reactions as described by Casey (2006) J. of Chem. Edu. Vol. 83, No. 2: 192-195, Lynn et al. (2000) J. Am. Chem. Soc. 122: 6601-6609, and Chen et al. (2003) Progress in Chemistry 15: 401-408.

Where the active agent is a low molecular weight compound or small molecule, a variety of techniques may be utilized to conjugate the low molecular weight compound or small molecule to a penetrating peptide as described herein, e.g., Click chemistry as described in Loh et al., Chem Commun (Camb), 2010 Nov. 28; 46(44):8407-9. Epub 2010 Oct. 7. See also, Thomson S., Methods Mol Med., (2004); 94:255-65, describing conjugation of small molecule carboxyl, hydroxyl, and amine residues to amine and sulfhydryl residues on proteins.

Methods are also available in the art for conjugating peptides to active agent carriers such as liposomes. See, for example, G. Gregoriadis (editor), Liposome Technology Third Edition, Volume II Entrapment of Drugs and Other materials into Liposomes, (2007), Informa Healthcare, New York, N.Y., which describes techniques for coupling peptides to the surface of liposomes. For the covalent attachment of proteins, to liposomes see, New, R.C.C., Liposomes: A Practical Approach, (1990) Oxford University Press Inc., N.Y. at pages 163-182.

C. Pharmaceutical Compositions

Pharmaceutical compositions including one or more skin penetrating peptides are provided. In some embodiments, the composition includes one or more skin penetrating peptides and one or more active agents. The pharmaceutical compositions can be administered by an suitable means, including, but not limited to, parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes of administration or using bioerodible inserts and can be formulated in dosage forms appropriate for each route of administration. However, it will be appreciated that the compositions are particularly suitable for topical delivery, preferably to the skin. Drugs can be formulated for immediate release, extended release, or modified release. A delayed release dosage form is one that releases a drug (or drugs) at a time other than promptly after administration. An extended release dosage form is one that allows at least a twofold reduction in dosing frequency as compared to that drug presented as a conventional dosage form (e.g. as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes but is not limited to diluents, binders, lubricants, desintegrators, fillers, and coating compositions.

“Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6^{th} Edition, Ansel et.al., (Media, Pa.: Williams and Wilkins, 1995) which provides information on carriers, materials, equipment and process for preparing tablets and capsules and delayed release dosage forms of tablets, capsules, and granules.

The compound can be administered to a subject with or without the aid of a delivery vehicle. Appropriate delivery vehicles for the compounds are known in the art and can be selected to suit the particular active agent. For example, in some embodiments, the active agent(s) is incorporated into or encapsulated by a nanoparticle, microparticle, micelle, synthetic lipoprotein particle, or carbon nanotube. For example, the compositions can be incorporated into a vehicle such as polymeric microparticles which provide controlled release of the active agent(s). In some embodiments, release of the drug(s) is controlled by diffusion of the active agent(s) out of the microparticles and/or degradation of the polymeric particles by hydrolysis and/or enzymatic degradation.

Suitable polymers include ethylcellulose and other natural or synthetic cellulose derivatives. Polymers which are slowly soluble and form a gel in an aqueous environment, such as hydroxypropyl methylcellulose or polyethylene oxide, may also be suitable as materials for drug containing microparticles or particles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before the all of the first agent is released. In still other embodiments, both agents are released at the same time over the same period of time or over different periods of time.

Pharmaceutical compositions can be prepared in unit dosage form or dosage units. As used herein “unit dosage form” or “dosage units”, refer to physically discrete units suitable as unitary dosages for human and animal subjects, each unit containing a predetermined quantity of skin penetrating peptide and/or active agent, each calculated in an amount sufficient to produce the desired effect in association with a pharmaceutically acceptable diluent, carrier or vehicle. The specifications for the unit dosage forms of the compositions depend on the particular skin penetrating peptide and/or active agent employed and the effect to be achieved, and the pharmacodynamics associated with each compound in the host.

Those of skill in the art will readily appreciate that dose levels can vary as a function of the specific compound, the nature of the delivery vehicle, and the like. Suitable dosages for a given compound are readily determinable by those of skill in the art by a variety of means.

1. Topical and Transdermal Formulations

The pharmaceutical composition can be a transdermal formulation, for example a gel, ointment, lotion, spray, or patch, all of which can be prepared using standard technology. Transdermal formulations can include one or more penetration enhancers in addition to the disclosed skin penetrating peptides.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) comprised of the drug dispersed or dissolved in a mixture of surfactant(s) and lipophillic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components.

Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

Additional agents that can be added to the formulation include penetration enhancers. In some embodiments, the penetration enhancer increases the solubility of the drug, improves transdermal delivery of the drug across the skin, in particular across the stratum corneum, or a combination thereof. Some penetration enhancers cause dermal irritation, dermal toxicity and dermal allergies. However, the more commonly used ones include urea, (carbonyldiamide), imidurea, N,N-diethylformamide, N-methyl-2-pyrrolidone, 1-dodecal-azacyclopheptane-2-one, calcium thioglycate, 2-pyrrolidone, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(10 oxyethylene ether), BRIJ® 78 (stearyl poly(20)oxyethylene ether), BRIJ® 96 (oleyl poly(10)oxyethylene ether), and BRIJ® 721 (stearyl poly (21) oxyethylene ether) (ICI Americas Inc. Corp.). Chemical penetrations and methods of increasing transdermal drug delivery are described in Inayat, et al., Tropical Journal of Pharmaceutical Research, 8(2):173-179 (2009) and Fox, et al., Molecules, 16:10507-10540 (2011). In some embodiments, the penetration enhancer is, or includes, an alcohol such ethanol, or others disclosed herein or known in the art.

Controlled release transdermal devices rely for their effect on delivery of a known flux of drug to the skin for a prolonged period of time, generally a day, several days, or a week. Two mechanisms are used to regulate the drug flux: either the drug is contained within a drug reservoir, which is separated from the skin of the wearer by a synthetic membrane, through which the drug diffuses; or the drug is held dissolved or suspended in a polymer matrix, through which the drug diffuses to the skin. Devices incorporating a reservoir will deliver a steady drug flux across the membrane as long as excess undissolved drug remains in the reservoir; matrix or monolithic devices are typically characterized by a falling drug flux with time, as the matrix layers closer to the skin are depleted of drug. Usually, reservoir patches include a porous membrane covering the reservoir of medication which can control release, while heat melting thin layers of medication embedded in the polymer matrix (e.g., the adhesive layer), can control release of drug from matrix or monolithic devices. Accordingly, the active agent can be released from a patch in a controlled fashion without necessarily being in a controlled release formulation.

Patches can include a liner which protects the patch during storage and is removed prior to use; drug or drug solution in direct contact with release liner; adhesive which serves to adhere the components of the patch together along with adhering the patch to the skin; one or more membranes, which can separate other layers, control the release of the drug from the reservoir and multi-layer patches, etc., and backing which protects the patch from the outer environment.

Common types of transdermal patches include, but are not limited to, single-layer drug-in-adhesive patches, wherein the adhesive layer contains the drug and serves to adhere the various layers of the patch together, along with the entire system to the skin, but is also responsible for the releasing of the drug; multi-layer drug-in-adhesive, wherein which is similar to a single-layer drug-in-adhesive patch, but contains multiple layers, for example, a layer for immediate release of the drug and another layer for control release of drug from the reservoir; reservoir patches wherein the drug layer is a liquid compartment containing a drug solution or suspension separated by the adhesive layer; matrix patches, wherein a drug layer of a semisolid matrix containing a drug solution or suspension which is surrounded and partially overlaid by the adhesive layer; and vapor patches, wherein an adhesive layer not only serves to adhere the various layers together but also to release vapor. Methods for making transdermal patches are described in U.S. Pat. Nos. 6,461,644, 6,676,961, 5,985,311, and 5,948,433.

2. Other Formulations

The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

3. Devices

In some embodiments, one or more of the penetrating peptide compositions of the present disclosure may be incorporated into a medical device known in the art, for example, drug eluting stents, catheters, fabrics, cements, bandages (liquid or solid), biodegradable polymer depots and the like. In some embodiments, the medical device is an implantable or partially implantable medical device.

### III. Methods of Use

Methods of using skin penetrating peptides and pharmaceutical compositions thereof are provided. Typically, one or more skin penetrating peptides is administered to a subject in need thereof in combination with one or more active agents. The term “combination” or “combined” is used to refer to either concomitant, simultaneous, or sequential administration of two or more agents. Therefore, the combinations can be administered either concomitantly (e.g., as an admixture), separately but simultaneously (e.g., via separate intravenous lines into the same subject, or locations of topical administration), or sequentially (e.g., one of the compounds or agents is given first followed by the second). The additional therapeutic agents can be administered locally or systemically to the subject, or coated or incorporated onto, or into a device or graft.

Effective amounts of skin penetrating peptide and active agent, suitable delivery vehicles, and protocols can be determined by conventional means. For example, in the context of therapy a medical practitioner can commence treatment with a low dose of one or more compositions in a subject or patient in need thereof, and then increase the dosage, or systematically vary the dosage regimen, monitor the effects thereof on the patient or subject, and adjust the dosage or treatment regimen to maximize the desired therapeutic effect. Further discussion of optimization of dosage and treatment regimens can be found in Benet et al., in Goodman & Gilman's The Pharmacological Basis of Therapeutics, Ninth Edition, Hardman et al., Eds., McGraw-Hill, New York, (1996), Chapter 1, pp. 3-27, and L. A. Bauer, in Pharmacotherapy, A Pathophysiologic Approach, Fourth Edition, DiPiro et al., Eds., Appleton & Lange, Stamford, Conn., (1999), Chapter 3, pp. 21-43, and the references cited therein.

The dosage levels and mode of administration will be dependent on a variety of factors such as the penetrating peptides used, the active agent, the context of use (e.g., the patient to be treated), and the like. Optimization of modes of administration, dosage levels, and adjustment of protocols, including monitoring systems to assess effectiveness are routine matters well within ordinary skill.

In the most preferred embodiments, the compositions are applied topically to the skin. As used herein, “topical administration” means application a body surfaces such as the skin or mucous membranes. Medications administered topically can be used to treat a wide range of ailments via a large range of classes including but not limited to creams, foams, gels, lotions, and ointments as discussed in more detail above. The composition can be application epicutaneously, meaning it is applied directly to the skin. Topical medications can also be inhaled (e.g., pulmonary delivery) or applied to the surface of tissues other than the skin, such as eye drops, or ear drops placed in the ear, or medications applied to the surface of a tooth, nasal canal, or vaginal or rectal mucosa. A topical effect, in the pharmacodynamic sense, may refer to a local or systemic, target for a medication.

Skin is the soft outer covering of vertebrates. Accordingly, the subject is most typically a vertebrate. For example, the subject can be a mammal, or a non-mammal such as a fish, reptile, amphibian, or bird. The skin of reptiles in comparison to that of amphibians and other endotherm amniotes is discussed in Alibardi, et al., J. Exp. Zool., 298B: 12-41 (2003) doi: 10.1002/jez.b.24. In some embodiments, subject is a human or a non-human primate.

A preferred formulation is a sustained release formulation, most preferably in an easy to administer topical formulation which enhances prolonged delivery and uptake of the compositions to the skin. The formulations may be applied topically to the skin, or adjacent to the site where therapy is desired, or injected or implanted within a sponge or other materials for use as a bulking agent which forms tissue in place of the implant. The formulation provides an effective amount of the active agent over the necessary time to improve the disease, disorder, or condition.

In some embodiments, the skin penetrating peptide is administered to the skin of a subject in combination with an active agent in an effective amount to increase the amount to active agent that crosses the stratum corneum relative to administration of the active agent of the subject in the absence of the skin penetrating peptide.

In some embodiment, the compositions are administered topically to achieve systemic circulation. In some embodiments, the compositions are administered topically to produce a regional or local effect with lower systemic drug levels than when an effective amount is administered systemically. More specifically, topical administration can result in local, regional, or systemic delivery of the active agent. For example, local, topical delivery means the active agent is delivered to the surface of the skin and to the tissue immediately below the surface of the skin. Regional, topical delivery means the active agent is delivered to the general application site (typically the skin) and it's interrelated surrounding tissues. Systemic, topical delivery generally means the active agent is delivered to circulatory system and regions outside the spaces described above.

In some embodiments the compositions are incorporated into or coated on an implant or graft and implanted in a patient's body. Implantation may be by surgical means or minimally invasive means such as a catheter or by injection or infusion into a tissue.

In one embodiment, the compositions are used for treating a subject with a dermatological disease, disorder, or condition. For example, the compositions can be used to treating abrasion of the outer layer or epidermis to, for example, smooth or blend scars, blemishes, or other skin conditions that may be caused by, for example, acne, sun exposure, and aging. In various embodiments, the compositions are used in treating wounds, impaired or damaged tissue, inflammatory conditions, and hypersensitivity, including skin irritation (e.g., itchy skin). Inflammation, hypersensitivity, and irritated skin may be associated with many conditions including atopic dermatitis, psoriasis, allergic reaction, skin fungus, among others including dermatosis, rosacea, skin infection, skin allergy, psoriasis, or acne, surgical/open wounds, skin pathogens (e.g., for bacteria, mycoplasmas, virus, fungi, prions), skin fungi, psoriasis, athlete's foot, traumatic wounds, acute and chronic infections, pressure ulcers, derma- abrasion, debrided wounds, laser re-surfacing, donor sites/grafts, exuding partial and full thickness wounds, and superficial injuries such as lacerations, cuts, abrasions, and minor skin irritations.

In some embodiments, the condition is sarcoidosis, psoriasis, pemphigus, erythema multiforme, atopia, dermatitis herpetiformis, or bullous disease of the skin, which may result from an autoimmune condition. For example, dermatitis herpetiformis is a skin manifestation associated with celiac disease, and affected subjects present with bumps, blisters, and itch. In other embodiments, the subject has an immune deficiency, such as Selective Immunoglobulin M Deficiency, which can manifest in part as a chronic dermatitis. In some embodiments, the inflammatory reaction is a systemic autoimmune reaction that involves itching and/or hives. In some embodiments, the inflammatory condition involves type IV hypersensitivity, including in some embodiments, one or more of Sjogrren's Syndrome, Sarcoidosis, or contact dermatitis.

In some embodiments, the compositions are administered to an effective area to treat impaired or damaged tissue, or inflammation associated with said impaired or damaged tissue. In some embodiments, the disease results at least in part from a hereditary defect of the skin or connective tissue, such as Dystrophic Epidermolysis Bullosa, Hisrontic Ectodermal Dysplasia (HED), or palmoplantar hyperkeratosis. Subjects with Dystrophic Epidermolysis Bullosa exhibit blisters of the skin and mucosal membranes and which result in substantial pain and itch HED subjects are often exhibit by partial or total alopecia, dystrophy of the nails, and hyperpigmentation of the skin (especially over the joints).

Palmoplantar hyperkeratosis (or Palmoplantar keratoderma) can manifest as an even, thick hyperkeratosis over the whole of the palm and sole. In these embodiments, the hypochlorous acid can relieve itch and discomfort from the disorder, and may provide general relief from symptoms and reduce the severity of disease.

In some embodiments, the composition is administered to a human or animal for skin pathogen disinfection, including bacteria, mycoplasmas, virus, or fungi, including skin fungi such as athlete's foot.

In still other embodiments, the compositions are administered to combat itch, where there is no discernible (e.g., objective) inflammatory reaction or irritant. For example, such condition may result from sensitive skin in combination with physical factors (such as ultraviolet radiation, heat, cold, wind), general chemical stress (e.g., cosmetics, soap, water, pollution), physiological stress or disorder, substance abuse, hormonal conditions (e.g., menstrual cycle), or other systemic malady. Even in the absence of an objective perception of skin inflammation, the compositions can be useful for reducing the subjective stinging, burning, warmth and tightness associated with itch (e.g., pruritus).

In some embodiments the conditions is blisters, calluses, corns, cellulite, dandruff, dermatitis herpetiformis, dermatographia, dry skin (xerosis), epidermoid cysts (sebaceous cysts), epidermolysis bullosa, erythema nodosum, granuloma annulare, henoch-schonlein purpura, ichthyosis, ichthyosis vulgaris, intertrigo, keratosis pilaris, lichen nitidus, lichen planus, lichen striatu, mastocytosis, morgellons disease, pityriasis rosea, seborrheic dermatitis, seborrheic keratosis, stasis dermatitis, Stevens-Johnson Syndrome , or Sweet's Syndrome.

In some embodiments, particularly when the active agent is or includes CsA, the disease or disorder is graft verse host disease and/or prevention of organ or tissue rejection, rheumatoid arthritis or a related diseases, psoriasis, dry eyes, nummular keratitis particularly following adenoviral keratoconjunctivitis, atopic dermatitis, Kimura disease, pyoderma gangrenosum, chronic autoimmune urticaria, acute systemic mastocytosis, acute severe ulcerative colitis, or posterior or intermediate uveitis.

In addition to treatment methods and other in vivo uses, the disclosed compositions disclosed can also be used in the context of ex vivo therapy and in vitro experimentation. For example, the skin penetrating peptides disclosed herein may be used to deliver any of a wide variety of active agents as discussed herein, as well as potential active agents, into viable cells ex vivo to, for example, effect a change in the cell, or in vitro to, for example, determine the potential therapeutic effect, toxicity, etc. of the active agent or potential active agent. For this reason, the skin penetrating peptides and penetrating peptide compositions of the present disclosure may be useful in the context of drug testing and/or screening.

In some embodiments, skin penetrating peptide compositions are used in ex vivo or in vitro gene silencing experiments, e.g., by introducing a penetrating peptide-interfering RNA conjugate directed to a gene target and optionally monitoring the effect on gene expression.

Additional ex vivo and in vitro uses include the use of skin penetrating peptides as disclosed herein conjugated or associated with one or more labeling agents (e.g., fluorescent agents or radioactive labels) or one or more labeling agent carriers in order to label viable cells.

### IV. Methods of Identifying Skin Penetrating Peptides

Methods of identifying skin penetrating peptides are provided. The methods typically include screening a library in silico to identify peptides that bind to a skin protein, preferably a structural protein of the skin such, for example, a keratin, collagen, a plectin, actin, or tubulin. In some embodiments, the method includes screening a library in silico to identify peptides that bind to an active agent of interest. In the most preferred embodiments, screen includes identifying peptides that bind to both a skin protein and an active agent of interest. The peptide library can be screened for binding to the active agent before or after screening binding to the skin protein. Likewise, the peptide library can be screen for binding to the skin protein before or after screening for binding to the active agent. Therefore, in some embodiments, the methods include two sequential screens wherein the library is first screened for binding to a skin protein and subsequently the peptides that are identified as binding to the skin protein are subjected to a second screen for binding an active agent. Alternatively, in some embodiments, include two sequential screens wherein the library is first screened for binding to an active agent and subsequently the peptides that are identified as binding to the active agent are subjected to a second screen for binding a skin protein. The screen can also be repeated for binding to two, three, or more skin proteins, two, three, or more active agents, or any combination thereof. For example, the screen carried out in the working Example below included a first step aims of keratin-binding sequences, followed by a second one selects, among such leads, those that show affinity for CsA as well.

As discussed in more detail below, the methods typically include screening the virtual peptide library or libraries for binding against the crystal structure of the skin protein and/or active agent. In the most preferred embodiments, the crystal structure and the associated molecular coordinate data are already available. However, in some embodiments, the crystal structures and coordinate data are prepared by the practitioner by, for example, solving the crystal structure of the protein or active agent of interest.

Primary molecular information for solved crystal structures of biological molecules can be stored and accessed in coordinate files. These files can list the atoms in each protein, and their 3D location in space, and can be available in several formats (PDB, mmCIF, XML). A typical PDB formatted file includes a large “header” section of text that summarizes the protein, citation information, and the details of the structure solution, followed by the sequence and a long list of the atoms and their coordinates. The archive can also contain the experimental observations that are used to determine these atomic coordinates. Publically available resources for coordinate files include, for example the RCSB Protein Data Bank.

A. Peptide Libraries

1. Content

Peptide libraries for screening can be prepared in silico using any suitable method known in the art. The peptide library can include peptides having “n” amino acids, wherein “n” is an integer between 2 and 100 inclusive, however, peptides in the library are most typically between about 3 and 30 amino acids (inclusive) in length, preferably between about 4 and about 20 amino acids (inclusive) in length. In the most preferred embodiments, the peptides are between about 5 and about 10 amino acids (inclusive) in length. The library can have peptides that are homogeneous or heterogeneous in length. By way of non-limiting example, the library can include peptides having a length of 4, 5, 6, 7, 8, 9, or 10; or the combination, or any sub-combination thereof.

The peptide library can be constructed by randomization of the sequences X1-Xn (wherein each “X” is independently any amino acid, or a specific sub-set thereof, and wherein “n” is an integer between 2 and 100 inclusive). In this way, the library can include a peptide for every possible combination of amino acid sequences for the designated length(s) of the peptides.

In some embodiments, the library includes additional “rules” that reduce the total number of peptides in the library. For example, a rule can require that all of the peptides include a cysteine in N-terminal half of the peptide and a cysteine in the C-terminal half of the peptide. In some embodiments, the peptide includes only two cysteines, one in N-terminal half of the peptide and one in the C-terminal half of the peptide (also referred to herein as “paired cysteines”). Accordingly, in such embodiments, the non-cysteine amino acids are most typically randomized among the remaining 19 canonical amino acids.

In some embodiments, the cysteines are positioned such that the resulting peptides form cyclic peptides via a disulfide bond under physiological conditions. In some embodiments the paired cysteines are equidistant from the N-terminus and C-terminus. For example, the paired cysteines are the N-terminal and C-terminal residues, or one residue each from N-terminus and the C-terminus, or two residues each from N-terminus and the C-terminus, or three residues each from N-terminus and the C-terminus, or four residues each from N-terminus and the C-terminus, or five residues each from N-terminus and the C-terminus, or six residues each from N-terminus and the C-terminus, or seven residues each from N-terminus and the C-terminus, or eight residues each from N-terminus and the C-terminus, or nine residues each from N-terminus and the C-terminus, or ten residues each from N-terminus and the C-terminus, etc. In some embodiments, the N-terminal residues are an Ala-Cys, the C-terminal residues are a Cys-Gly. In some embodiments, the paired cysteines have an integer between 1 and 98 amino acids between them.

Two examples are illustrated below:

```
(SEQ ID NO: 16)
Cys - X1 - Xn - Cys
    |  |
        |_____S-S____|
or
(SEQ ID NO: 17)
Ala - Cys - X1 - Xn - Cys - Gly
                |  |
                    |_____S-S____|
```

Other rules include, for example, removal of peptides that have less than “z” different amino acids; more than two consecutive equal amino acids; more than “z” aliphatic amino acids (Ala, Val, Leu, and Ile); “z” aromatic amino acids (Phe, Tyr, and Trp); less than “z” charged amino acids

(Lys, Arg, His, Asp, and Glu); more than “z” charged amino acids (Lys, Arg, His, Asp, and Glu); only alternated hydrophobic and charged amino acids; wherein “z” is an integer between 2 and 100 inclusive, but longer than the “n” length of the peptide. “Z” can be different for each rule. Any of the rules disclosed herein or otherwise devised by a practitioner can be used alone or in any combination or sub-combination to achieve the desired library size and content.

An exemplary peptide library discussed in more detail in the working Examples below. Briefly, the list of sequences A-C—X1-Xn-C-G (n=5, 6, 7, and 8) (SEQ ID NO:22) including all possible combinations of all natural amino acids, excluding cysteine, was generated using a library generator code developed in Java. A preliminary syntactic screening of the library was performed to eliminate sequences containing: a) less than four different amino acids and more than two consecutive equal amino acids, b) more than three aliphatic amino acids (Ala, Val, Leu, and Ile) and/or two aromatic amino acids (Phe, Tyr, and Trp), c) less than one and more than three charged amino acids (Lys, Arg, His, Asp, and Glu), d) only alternated hydrophobic and charged amino acids.

In the exemplary screen discussed in more detail below, four virtual libraries (pentamers, hexamer, heptamer, and octamer) of disulfide-bonded peptides were constructed and screened against the crystal structures of keratin and CsA, available on RCSB Protein Data Bank (PDB IDs: 3TNU and 1CSA, respectively).

2. Coordinate Files

After a peptide library is prepared, coordinate files of the peptides (SPPs) are generated or obtained. Methods, software, and systems for preparing coordinate files are known in the art and include using, for example, the open source graphic chemical structure visualization programs PyMOL or Avogadro. Once the coordinate file is prepared, “active residues” can be defined. “Active residues” are amino acids from the putative skin penetrating peptide, the skin protein, and active agent that screened for interaction as discussed in more detail below. All amino acids in the peptide can be designated as active residues, or a user defined subset or specific amino acid(s) can be defined as active residues. For example, rules can be applied to reduce the number of active residues that need to be screened. In the exemplary screen described below, the randomized region of every peptide including the residues framed by cysteines, was defined as active.

B. Selection of One or More Binding Partners

1. Skin Proteins

The methods can include screening the peptides in a peptide library for binding to one or more skin proteins in silico. Skin proteins are discussed in more detail above. In some embodiments, the skin protein is a keratin, preferably a keratin that is prominent in stratified epithelium. For example, in particular embodiments the skin protein is keratin 5, keratin 14, or a combination thereof.

Next, a coordinate file for the skin protein(s) of interest is prepared or obtained. The coordinate file can also be for a complex or other macromolecular structure including the skin protein of interest. By way of non-limiting example, in some embodiments, the coordinate file for a keratin pair is a prepared. In the Examples below, the coordinate file was prepared for the human keratin 5 and keratin 14 pair. Coordinate files can be obtained from the RCSB Protein Data Bank (PDB ID: 3TNU).

The coordinate file can be further defined by the practitioner. For example, active residues can be defined. As discussed above for the peptide library, all amino acids can be designated as active residues, or a user defined subset or specific amino acid(s) can be defined as active residues. For example, in the Examples below, the solvent accessible residues on keratin were defined as active residues and used as a target for ligand docking. All active residues exhibit a relative solvent accessibility higher than 40%, as defined by the program NACCESS [Capra, et al., Bioinformatics, 23(15):1875-1882 (2007)]. 2. Active Agents

The methods can include screening the peptides in a peptide library for binding to one or more active agents in silico. Active agents are discussed in more detail above. In preferred embodiments, the active agent is one can be therapeutic to a subject in need thereof when delivered transdermally into or through the skin.

Once the active agent is selected by the practitioner, a coordinate file for the active agent is prepared. The coordinate file can also be for a complex or other macromolecular structure including the active agent. In the Example below, the coordinates for Cyclosporine A were obtained from the RCSB Protein Data Bank (PDB ID: 1CsA).

The coordinate file can be further defined by the practitioner. For example, active residues can be defined. As discussed above for the peptide library and skin protein, all amino acids can be designated as active residues, or a user defined subset or specific amino acid(s) can be defined as active residues. In the working Example below, All residues of CsA were defined as active residues.

C. In Silico Screening

Next, active residues of the peptides from the library are virtually aligned, bound, or docked to the active residues of the skin protein or active agent. In preferred embodiments, each active residue from each of the peptides in the library is analyzed against each of the residues of the skin peptide or active agent.

The peptide library can be screened against the skin peptide or active using suitable in silico means known in the art. In preferred embodiments, including the working Example below, the software program HADDOCK (version 2.1) is utilized [Dominguez, et al., J Am Chem Soc, 125(7):1731-1777 (2003); de Vries, et al., Proteins, 69(4):726-733 (2007)]. HADDOCK simulates protein-peptide interaction and estimates, through external software, the free energy of binding in solution [Dominguez, et al., J Am Chem Soc, 125(7):1731-1777 (2003)]. Parameters (e.g., temperatures for heating/cooling steps and number of molecular dynamics sets per stage) can be defined by the practitioner. In some embodiments, the default parameters are utilized.

The resulting docked structures can be grouped in clusters. Their binding energy can be averaged. In a particular embodiment, the producer described in [Menegatti, et al., Chemical and Biomolecular Engineering, North Carolina State University: Raleigh. p. 419 (2013)], which is specifically incorporated by reference herein in its entirety, is utilized. Briefly, the clusters are analyzed using built-in scoring functions, which include empirical scoring functions that estimate the free energy of binding, and hence the affinity, of a given protein-ligand complex of known three-dimensional structure. These functions account for van der Waals interactions, hydrogen bonding, deformation penalty, and hydrophobic effects, atomic contact energy, softened van der Waals interactions, partial electrostatics, and additional estimations of the binding free energy, and dipole-dipole interactions. The rankings can then by compiled, each listing the peptide sequences ordered based on the scoring value obtained according to the respective function [Wang, et al., J Med Chem, 46(12): 2287-2303 (2003); Mashiach, et al., Nucleic Acids Res, 36(Web Server issue): W229-W232 (2008)]. These rankings can be compiled and averaged to obtain a final list of putative skin penetrating peptide sequences.

After each round of screening, a threshold number of peptides can be selected for another round of in silico screening against a different target, or for further in vitro or in vivo testing. The threshold number can be an absolute number or a percentage of the total number of peptides in the library. For example, the user can select an integer from 1 to 10,000 (inclusive) or more peptides beginning from the top of the ranked list. In another example, the user can select the top percentage of peptides ranging from 1% to 50%. In specific embodiments, the user selects the top 50%, 25%, 10%, 5%, or 1% of peptides for further analysis. In some embodiment, the peptides are selected based on the predicted dissociation constant with the skin protein. In particular embodiments, peptide is selected if the predicted dissociation constant is between about 10⁻³ M and 10⁻⁸ M. In the Example below, the top 5% sequences in docking to keratin 14 and 5 were in turn docked against CsA.

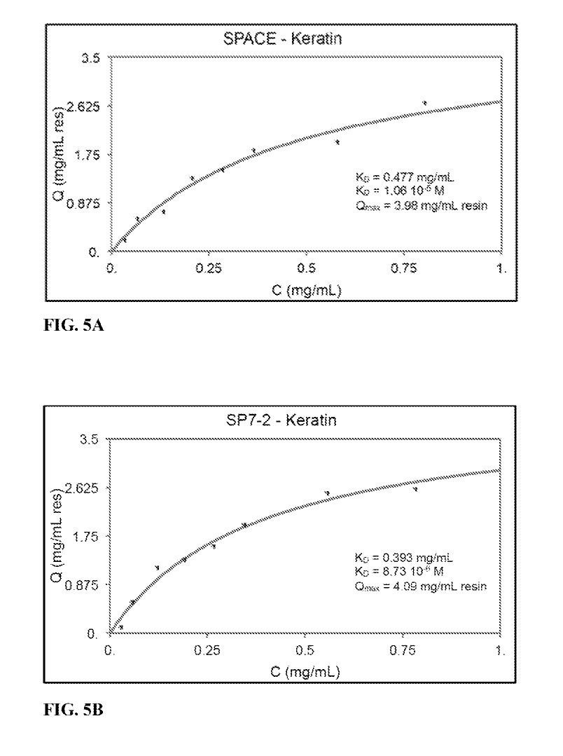

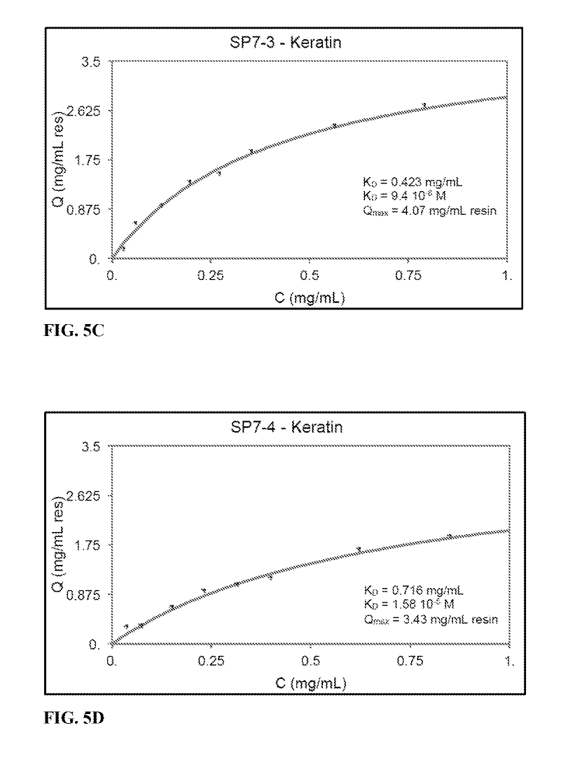

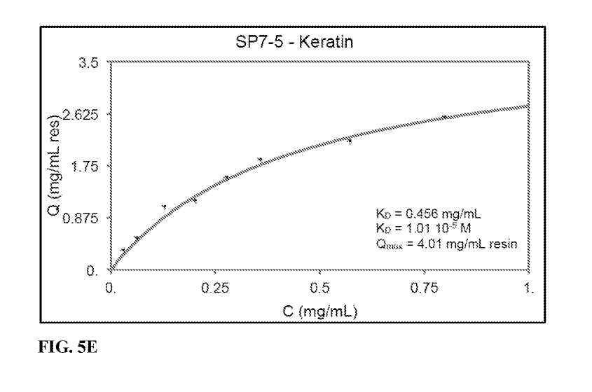

Assays to evaluate the binding of a given peptide for a skin protein (e.g., keratin) (e.g., tests confirming the in silico screening results) can be carried out in vitro or in vivo. Suitable assays and experiments are known in the art and exemplary methods are described below. For example, binding assays can be carried out between each selected peptide and one or more of its targets (e.g., a skin protein such as keratin). In some embodiments, two or more of the targets (e.g., a skin protein such as keratin, and an active agent) are present in the same binding experiment. Binding experiments and assays for investigating molecule-molecule interactions are well known in the art and include, for example, bimolecular fluorescence complementation, affinity electrophoresis, label transfer, yeast two-hybrid, immunoprecipitation, affinity chromatography, surface plasmon resonance, FRET, Biacore assay, etc. (e.g., see examples section below, e.g., see example 2, FIG. 2 and FIG. 5A-5E).

Experiments can also be carried out in vitro or in vivo to determine the skin penetration and/or toxicity of the peptide, preferably when administered to skin in combination with an active agent of interest. Skin testing assays include, for example dermal penetration testing (also known as percutaneous penetration) which can be used to measure the absorption or penetration of a substance through the skin barrier and into the skin and/or circulation, dermal toxicity testing which can be used to determine the local or systemic effects of peptide preferably in combination with an active agent (OECD. (2004). Guidance Document for the Conduct of Skin Absorption Studies. OECD Series on Testing and Assessment. No. 28. OECD. Paris, France). Protocols for suitable assays are known in the art, and examples are provided below.

Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure (Set A, numbered 1-42; and Set B numbered 1-54) are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspects, Set A
1. A polypeptide consisting of between 3 and 100 amino acids, wherein the polypeptide binds to a skin protein with a Kd of between about 10⁻³ M and about 10⁻⁸ M, wherein the polypeptide is not a full-length naturally occurring protein, and wherein the polypeptide does not comprise the amino acid sequence of SEQ ID NO:1 (SPACE™), SEQ ID NO:23 (TD-1), SEQ ID NO:24 (Poly-R), SEQ ID NO:14 (DLP), or SEQ ID NO:25 (LP-12).
2. The polypeptide of 1, wherein the polypeptide consists of between 3 and 10 amino acids inclusive.
3. The polypeptide of any one of 1-2, wherein the skin protein is selected from the group consisting of keratin, collagen, plectin, actin, and tubulin.
4. The polypeptide of any one of 1-3, wherein the skin protein is keratin.
5. The polypeptide of 4, wherein the keratin is keratin 5, keratin 14, or a combination thereof
6. The polypeptide of any one of 1-5, wherein the polypeptide comprises or consists of Cys-X1-Xn-Cys (SEQ ID NO:16), or Ala-Cys-X1-Xn-Cys-Gly (SEQ ID NO:17), wherein each “X” is independently any amino acid, or a subset thereof for example the 19 canonical amino acids excluding cysteine; wherein “n” is 0 or an integer between 1 and 100 inclusive; and wherein peptide cyclization is achieved through the formation of a disulfide bond between two cysteines.
7. The polypeptide of 6, wherein n is 0, 1, 2, 3, 4, 5, 6, 7, 8, 9, or 10.
8. The polypeptide of any of 1-7, wherein the amino acid sequence comprises one or more sequence motifs selected from the group consisting of NHN, QHN, NRN, and QRQ.
9. The polypeptide of any of 1-6, wherein the polypeptide comprises or consists of the amino acid sequence cyclo[C—X1-X2-X3-X4-X5-C] (SEQ ID NO:18), wherein “C” is a Cysteine; “X1” is Serine, Threonine, Asparagine, Glutamine, or Glycine; “X2” is Histidine, Asparagine, or Glutamine; “X3” is Histidine, Arginine, Asparagine, or Glutamine; “X4” is Histidine, Asparagine, Glutamine, Serine, or Threonine; “X5” is Serine, Threonine, Glycine, or Alanine; and wherein peptide cyclization is achieved through the formation of a disulfide bond between two cysteines.
10. The polypeptide of any of 1-6, wherein the polypeptide comprises or consists of the amino acid sequence cyclo[C—X1-X2-X3-X4-X5-X6-C] (SEQ ID NO:19), wherein “C” is Cysteine; “X1” is Serine, Threonine, Asparagine, Glutamine, or Glycine; “X2” is Serine, Threonine, Asparagine, or Glutamine; “X3” is Histidine, Arginine, Lysine, Asparagine, Glutamine, Glycine, or Alanine; “X4” is Serine, Threonine, Asparagine, Glutamine, Glycine, or Arginine; “X5” is Histidine, Arginine, Lysine, Asparagine, Glutamine, Serine, or Threonine; “X6” is Asparagine, Glutamine, Serine, Threonine, Arginine, Glycine, or Alanine; and wherein peptide cyclization is achieved through the formation of a disulfide bond between two cysteines.
11. The polypeptide of any of 1-6, wherein the polypeptide comprises or consists of the amino acid sequence cyclo[C—X1-X2-X3-X4-X5-X6-X7-C] (SEQ ID NO:20), wherein “C” is a Cysteine; “X1” is Serine, Threonine, Glycine, Alanine, or Valine; “X2” is Glycine, Alanine, Valine, Leucine, Serine, or Threonine; “X3” is Glycine, Alanine, Serine, or Threonine; “X4” is Asparagine, Glutamine, Arginine, or Lysine; “X5” is Histidine, Asparagine, Glutamine, Tryptophan, Serine, or Threonine; “X6” is Serine, Threonine, Histidine, Asparagine, Glutamine, Glycine, or Alanine; “X7” is Serine, Threonine, Histidine, Asparagine, Glutamine, Glycine, or Alanine; and wherein peptide cyclization is achieved through the formation of a disulfide bond between two cysteines.
12. The polypeptide of any of 1-6, wherein the polypeptide comprises or consists of the amino acid sequence cyclo[C—X1-X2-X3-X4-X5-X6-X7-X8-C] (SEQ ID NO:21), wherein “C” is a Cysteine; “X1” is Serine, Threonine, Asparagine, Glutamine, Glycine, or Alanine; “X2” is Alanine, Serine, Threonine, or Arginine; “X3” is Histidine, Asparagine, Glutamine, Lysine, or Arginine; “X4” is Asparagine, Arginine, Histidine, or Tryptophan; “X5” is Glycine, Alanine, Arginine, Glutamine, Lysine, or Arginine; “X6” is Histidine, Tryptophan, Glycine, or Alanine; “X7” is Serine, Threonine, Asparagine, or Glutamine; “X8” is Serine, Threonine, Glycine, or Alanine; and wherein peptide cyclization is achieved through the formation of a disulfide bond between two cysteines.
13. The polypeptide of any of 1-6, wherein the polypeptide comprises or consists of the amino acid sequence of SEQ ID NO: 2, 3, 4, 5, 6, 7, 8, 9, 10, 11, 12, or 13.
14. The polypeptide of any of 1-6, wherein the polypeptide binds to a pharmaceutically or therapeutically active agent.
15. The polypeptide of 14, wherein the polypeptide increases absorption or penetration of the active agent into one or more tissues or cells of the skin compared to absorption or penetration of the active agent in the absence of the polypeptide, when the polypeptide and the active agent are administered in combination to the skin of a mammalian subject.
16. The polypeptide of 14, wherein the polypeptide increases delivery of the active agent across the stratum corneum when the polypeptide and the active agent are administered in combination to the skin of a subject.
17. The polypeptide of 14, wherein the active agent is Cyclosporine A.
18. A pharmaceutical composition comprising the polypeptide of any one of 1-17.
19. The pharmaceutical composition of 18 further comprising an active agent.
20. A method of treating a subject in need thereof comprising administering to the subject the polypeptide of any one of 1-17 in combination with an active agent.
21. The method of 20, wherein the polypeptide and the active agent are together in the same pharmaceutical composition.
22. The method of 20, wherein the polypeptide and the active agent are in separate pharmaceutical compositions.
23. The method of any one of 20-22, wherein the polypeptide and active agent are administered topically to the subject.
24. The method of 23, wherein the polypeptide and active agent are administered topically to the skin of the subject.
25. The method of 24, wherein the polypeptide is administered in an effective amount to increase absorption or penetration of the active agent into the skin.
26. The method of 25, wherein the polypeptide is administered in an effective amount to increase delivery of the active agent across the stratum corneum, compared to administering the active agent in the absence of the polypeptide.
27. The method of any one of 20-26, wherein active agent is a polypeptide, nucleic acid, or small molecule.
28. The method of any one of 20-26, wherein the active agent is a dermatological agent.
29. The method of any one of 20-26, wherein the active agent is cyclosporine A.
30. The method of any one of 20-29, wherein the subject has a dermatological condition, disease, or disorder.
31. The method of 30, wherein the active agent is administered in an effective amount reduce one or more symptoms associated with the dermatological condition, disease, or disorder.
32. A method of screening for skin penetrating peptides in silico comprising screening a virtual peptide library for binding to a skin protein by individually simulating binding of the active residues of each peptide's crystal structure to the active residues of the skin protein's residues, and selecting the peptide as a skin penetrating peptide if the predicted dissociation constant (Kd) is between about 10⁻³ M and 10⁻⁸M.
33. The method of 32, wherein peptide library comprises randomization of the sequences X1-Xn, wherein each “X” is independently any amino acid, or a specific sub-set thereof, and wherein “n” is an integer between 2 and 100 inclusive.
34. The method of 32 or 33, wherein the skin protein is selected from the group consisting of keratin, collagen, plectin, actin, and tubulin.
35. The method of 34, wherein the skin protein is keratin.
36. The method of 35, wherein the keratin is keratin 5, keratin 14, or a combination thereof
37. The method of any one of 32-36, wherein the polypeptides of the peptide library comprise or consist of Cys-X1-Xn-Cys (SEQ ID NO:16), or Ala - Cys-X1-Xn-Cys-Gly (SEQ ID NO:17), wherein each “X” is independently any amino acid, or a subset thereof, such as the 19 canonical amino acids excluding cysteine; wherein “n” is 0 or an integer between 1 and 100 inclusive; and wherein peptide cyclization is achieved through the formation of a disulfide bond between two cysteines.
38. The method of 37, wherein n is 0, 1, 2, 3, 4, 5, 6, 7, 8, 9, or 10, or a combination thereof.
39. The method of any one of 32-38, wherein the peptide library excludes peptides that have less than “z” different amino acids; more than two consecutive equal amino acids; more than “z” aliphatic amino acids (Ala, Val, Leu, and Ile); “z” aromatic amino acids (Phe, Tyr, and Trp); less than “z” charged amino acids (Lys, Arg, His, Asp, and Glu); more than “z” charged amino acids (Lys, Arg, His, Asp, and Glu); only alternated hydrophobic and charged amino acids; or any combination thereof and wherein “z” is an integer between 2 and 100 inclusive, but longer than the “n” length of the peptide.
40. The method of any one of 32-39, further comprising screening the selected skin penetrating peptides for binding to active agent by individually simulating binding of the active residues of each peptide's crystal structure to the active residues of the active agent's residues, and selecting the peptide as a skin penetrating peptide if the predicted dissociation constant (Kd) is between about 10⁻³ M and 10⁻⁸ M.
41. The method of any one of 32-40, wherein the active residues of the skin protein are those that exhibit a relative solvent accessibility higher than 40%, as defined by the program NACCESS.
42. The method of any one of 32-41, wherein all of the residues of each peptide in the peptide library are active residues.

### Aspects, Set B

1. A polypeptide having a length in a range of from 5 to 100 amino acids, comprising the amino acid sequence X1-X2-X3-X4-X5-X6-X7-X8, wherein
   X1 is T, S, L, N, or V;
   X2 is G, A, L, S, V, or H;
   X3 is S, T, V, N, or H;
   X4 is T, L, V, N, Q, G, or H;
   X5 is Q, N, H, V, S, R, T, or A;
   X6 is absent or present, and if present: H, W, N, or R;
   X7 is absent or present, can only be present if X6 is present, and if present: Q, S, N, or A; and
   X8 is absent or present, can only be present if X6 and X7 are present, and if present: T,
      wherein the polypeptide: (i) binds to a skin protein with a Kd of between 10⁻³ M and 10⁻⁸ M, (ii) is not a full-length naturally occurring protein, and (iii) does not include the amino acid sequence TGSTQHQ (SEQ ID NO: 301).
2. The polypeptide of 1, wherein the amino acid sequence X1-X2-X3-X4-X5-X6-X7-X8 is flanked by C residues.
3. The polypeptide of 1 or 2, comprising the amino acid sequence C—X1-X2-X3-X4-X5-X6-X7-X8-C.
4. The polypeptide of 3, comprising the amino acid sequence AC—X1-X2-X3-X4-X5-X6-X7-X8-CG.
5. The polypeptide of any one of 2-4, wherein the polypeptide is a cyclic peptide and a disulfide bond is present between said cysteine residues.
6. The polypeptide of any one of 1-5, wherein:
   X1 is T, S, L, N, or V;
   X2 is G, A, L, S, or V;
   X3 is S, T, V, N, or H;
   X4 is T, L, V, N, Q, G, or H;
   X5 is Q, N, H, V, S, R, T, or A;
   X6 is H, W, N, or R;
   X7 is Q, S, N, or A; and
   X8 is absent or present, and if present: T.
7. The polypeptide of 6, wherein X1-X2-X3-X4-X5-X6-X7-X8 is NAHQARST (SEQ ID NO: 305).
8. The polypeptide of 6, comprising the sequence ACNAHQARSTCG (SEQ ID NO: 5).
9. The polypeptide of any one of 1-5, wherein:

X1 is T, S, L, N, or V;
X2 is G, A, L, S, V, or H;
X3 is S, T, V, or N;
X4 is T, L, V, N, Q, G, or H;
X5 is Q, N, H, V, S, R, or T;
X6 is absent or present, and if present: H, W, N;
X7 is absent or present, can only be present if X6 is present, and if present: Q, S, N, or A; and
X8 is absent.
10. The polypeptide of 9, wherein X7 is absent and X1-X2-X3-X4-X5-X6 is THTGRN (SEQ ID NO: 303).
11. The polypeptide of 9, comprising the sequence ACTHTGRNCG (SEQ ID NO: 3).
12. The polypeptide of 9, wherein X6 and X7 are absent and X1-X2-X3-X4-X5 is SHNHT (SEQ ID NO: 302).
13. The polypeptide of 9, comprising the sequence ACSHNHTCG (SEQ ID NO: 2).
14. The polypeptide of any one of 1-5, wherein:
   X1 is T, S, L, N, or V
   X2 is G, A, L, S, or V;
   X3 is S, T, or V;
   X4 is T, L, V, N, Q, or G;
   X5 is Q, N, H, V, S, or R;
   X6 is H, W, or N;
   X7 is Q, S, N, or A; and
   X8 is absent.
15. The polypeptide of 14, wherein X1-X2-X3-X4-X5-X6-X7 is selected from: SASQVHN (SEQ ID NO: 309), NGTGSHQ (SEQ ID NO: 310), SVTTQHQ (SEQ ID NO: 311), and VSVTNHQ (SEQ ID NO: 312).
16. The polypeptide of 14, comprising a sequence selected from:

(SEQ ID NO: 9)
ACSASQVHNCG,
(SEQ ID NO: 10)
ACNGTGSHQCG,
(SEQ ID NO: 11)
ACSVTTQHQCG,
and
(SEQ ID NO: 12)
ACVSVTNHQCG.

17. The polypeptide of any one of 1-5, wherein:
   X1 is T, S, or L;
   X2 is A, L, or S;
   X3 is S, T, or V;
   X4 is T, L, V, N, or G;
   X5 is Q, N, H, or R;
   X6 is H, W, or N;
   X7 is S, N, or A; and
   X8 is absent.
18. The polypeptide of 17, wherein X1-X2-X3-X4-X5-X6-X7 is selected from: SATLQHS (SEQ ID NO: 304), SLTVNWN (SEQ ID NO: 306), LSVNHNA (SEQ ID NO: 307), SASTNHN (SEQ ID NO: 308), and TSTGRNA (SEQ ID NO: 313).
19. The polypeptide of 17, comprising a sequence selected from:

(SEQ ID NO: 4)
ACSATLQHSCG,
(SEQ ID NO: 6)
ACSLTVNWNCG,
(SEQ ID NO: 7)
ACLSVNHNACG,
(SEQ ID NO: 8)
ACSASTNHNCG,
and
(SEQ ID NO: 13)
ACTSTGRNACG.

20. A polypeptide having a length in a range of from 5 to 100 amino acids, comprising the amino acid sequence set forth in any one of SE ID NOs: 326-417, 176-267, and 26-117, wherein the polypeptide: is not a full-length naturally occurring protein.
21. The polypeptide of 20, comprising the amino acid sequence set forth in any one of SE ID NOs: 176-267, and 26-117, wherein the polypeptide is a cyclic peptide and a disulfide bond is present between the two cysteine residues present in said sequence.
22. A polypeptide having a length in a range of from 5 to 100 amino acids, comprising the amino acid sequence C—X1-Xn-C (SEQ ID NO:16), or AC—X1- Xn-CG (SEQ ID NO:17), wherein each “X” is independently any amino acid excluding cysteine; wherein “n” is 2, 3, 4, 5, 6, 7, 8, 9, or 10; wherein a disulfide bond is present between the two cysteines; and wherein the polypeptide does not comprise the amino acid sequence set forth in any of SEQ ID NOs:1, 14, 23-25, 118-121, 301, 314, 323, and 418-421.
23. The polypeptide of 22, comprising one or more sequence motifs selected from the group consisting of NHN, QHN, NRN, and QRQ.
24. The polypeptide of any one of 1-23, wherein the skin protein is selected from the group consisting of keratin, collagen, plectin, actin, and tubulin.
25. The polypeptide of 24, wherein the skin protein is keratin.
26. The polypeptide of 25, wherein the keratin is keratin 5, keratin 14, or a combination thereof.
27. The polypeptide of any one of 1-26, having a length in a range of from 7 to 30 amino acids.
28. The polypeptide of 27, having a length in a range of from 7 to 12 amino acids.
29. The polypeptide of any one of 1-28, wherein the polypeptide binds to a therapeutically active agent.
30. The polypeptide of 29, wherein the polypeptide increases absorption or penetration of the therapeutically active agent into one or more tissues or cells of the skin compared to absorption or penetration of the therapeutically active agent in the absence of the polypeptide, when the polypeptide and the therapeutically active agent are administered in combination to skin of a mammalian subject.
31. The polypeptide of 29, wherein the polypeptide increases delivery of the therapeutically active agent across stratum corneum when the polypeptide and the therapeutically active agent are administered in combination to the skin of a subject.
32. The polypeptide of any one of 1-31, wherein the polypeptide is bound or conjugated to a therapeutically active agent.
33. The polypeptide of any one of 29-32, wherein the therapeutically active agent is a polypeptide, nucleic acid, or small molecule.
34. The polypeptide of any one of 29-32, wherein the therapeutically active agent is a dermatological agent.
35. The polypeptide of any one of 29-32, wherein the therapeutically active agent is Cyclosporine A.
36. A pharmaceutical composition comprising the polypeptide of any one of 1-35.
37. The pharmaceutical composition of 36, further comprising a therapeutically active agent.
38. The pharmaceutical composition of 36 or 37, wherein the polypeptide is bound or conjugated to the therapeutically active agent.
39. The pharmaceutical composition of any one of 36-38, wherein the therapeutically active agent is a polypeptide, nucleic acid, or small molecule.
40. The pharmaceutical composition of any one of 36-38, wherein the therapeutically active agent is a dermatological agent.
41. The pharmaceutical composition of any one of 36-38, wherein the therapeutically active agent is Cyclosporine A.
42. A method of treating a subject in need thereof comprising administering to the subject the polypeptide of any one of 1-35 in combination with a therapeutically active agent.
43. The method of 42, wherein the polypeptide and the therapeutically active agent are together in the same pharmaceutical composition.
44. The method of 43, wherein the polypeptide is bound or conjugated to the therapeutically active agent.
45. The method of 42, wherein the polypeptide and the therapeutically active agent are in separate pharmaceutical compositions.
46. The method of any one of 42-45, wherein the polypeptide and therapeutically active agent are administered topically to the subject.
47. The method of 46, wherein the polypeptide and therapeutically active agent are administered topically to the skin of the subject.
48. The method of 47, wherein the polypeptide is administered in an effective amount to increase absorption or penetration of the therapeutically active agent into the skin.
49. The method of 48, wherein the polypeptide is administered in an effective amount to increase delivery of the therapeutically active agent across the stratum corneum, compared to administering the therapeutically active agent in the absence of the polypeptide.
50. The method of any one of 42-49, wherein the therapeutically active agent is a polypeptide, nucleic acid, or small molecule.
51. The method of any one of 42-49, wherein the therapeutically active agent is a dermatological agent.
52. The method of any one of 42-49, wherein the therapeutically active agent is cyclosporine A.
53. The method of any one of 42-52, wherein the subject has a dermatological condition, disease, or disorder.
54. The method of 53, wherein the therapeutically active agent is administered in an effective amount reduce one or more symptoms associated with the dermatological condition, disease, or disorder.

### EXAMPLES

### Example 1

### Selection of SPPs for CsA Delivery Via in Silico Library Screening

### Materials and Methods

Abbreviations

SPP: skin penetrating peptide; SPACE™: skin penetrating and cell entering; CsA: Cyclosporine A; SC: stratum corneum; CPE: chemical penetration enhancer; PBS: phosphate buffer saline; FDC: Franz diffusion cells; HEKa: Human epidermal keratinocytes.

In Silico Library Screening

The list of sequences A-C—X1-Xn-C-G (n=5, 6, 7, and 8) including all possible combinations of all natural amino acids, excluding cysteine, was generated using a library generator code developed in Java. A preliminary syntactic screening of the library was performed to eliminate sequences containing: a) less than four different amino acids and more than two consecutive equal amino acids, b) more than three aliphatic amino acids (Ala, Val, Leu, and Ile) and/or two aromatic amino acids (Phe, Tyr, and Trp), c) less than one and more than three charged amino acids (Lys, Arg, His, Asp, and Glu), d) only alternated hydrophobic and charged amino acids. The coordinate files of the peptides (SPPs) were generated using the open source graphic chemical structure visualization program PyMOL [Seeliger, et al., J Comput Aided Mol Des, 24(5):417-422 (2010)].

The coordinate file for human keratin 5 and keratin 14 pair was obtained from the RCSB Protein Data Bank (PDB ID: 3TNU). The solvent accessible residues on keratin were defined as “active” and used as target for ligand docking. All active residues exhibit a relative solvent accessibility higher than 40%, as defined by the program NACCESS [Capra, et al., Bioinformatics, 23(15):1875-1882 (2007)].

The randomized region of every peptide, including the residues framed by cysteines, was defined as active. Each peptide in the library was docked against keratin using the software HADDOCK (version 2.1) [Dominguez, et al., J Am Chem Soc, 125(7):1731-1777 (2003); de Vries, et al., Proteins, 69(4):726-733 (2007)]. Default parameters, i.e. temperatures for heating/cooling steps and number of molecular dynamics sets per stage, were used in the docking procedure.

The resulting docked structures were grouped in clusters and their binding energy was averaged following the procedure discussed in [Menegatti, et al., Chemical and Biomolecular Engineering, North Carolina State University: Raleigh. p. 419 (2013)]. Briefly, the clusters were analyzed using built-in scoring functions, which comprise empirical scoring functions that estimate the free energy of binding, and hence the affinity, of a given protein-ligand complex of known three-dimensional structure. These functions account for van der Waals interactions, hydrogen bonding, deformation penalty, and hydrophobic effects, atomic contact energy, softened van der Waals interactions, partial electrostatics, and additional estimations of the binding free energy, and dipole-dipole interactions.

The rankings were then compiled, each listing the sequences ordered based on the scoring value obtained according to the respective function [Wang, et al., J Med Chem, 46(12): 2287-2303 (2003); Mashiach, et al., Nucleic Acids Res, 36(Web Server issue): W229-W232 (2008)]. These rankings were finally compiled and averaged to obtain a final list of sequences. The top 5% sequences were in turn docked against CsA by repeating the above procedure. The coordinates for Cyclosporine A were also obtained from the RCSB Protein Data Bank (PDB ID: 1CsA). All residues of CsA were defined as active for this docking. Peptide clustering and determination of binding energy were performed as described above.

### Results

Disulfide-bonded, cyclic heptapeptides, whose sequences are ACTGSTQHQCG (SEQ ID NO:1) and ACKTGSHNQCG (SEQ ID NO:14) respectively, were discovered via screening of a phage display library and subsequently characterized for their ability to deliver siRNA and CsA [Hsu, et al., Proc Natl Acad Sci USA, 108(38):15816-15821 (2011)]. Among the known SPPs, SPACE™ affords the highest enhancement of transdermal permeation of both drugs, while causing the lowest skin irritation or toxicity for keratinocytes.

To screen for new skin penetrating peptides, libraries were initially constructed as a FASTA format list obtained through randomization of the sequences X1-Xn (n=5, 6, 7, and 8), framed between Ala-Cys and Cys-Gly, comprising 19 out of the 20 natural amino acids. Cysteine was excluded, as it is specifically needed for peptide cyclization. Due to their high theoretical diversities, which respectively amount to approx. 2.48 10⁶, 4.7 10⁷, 8.94 10⁹, and 1.7 10¹⁰, the libraries cannot reasonably be constructed and accurately screened in silico in their entirety. Therefore, before producing the coordinate files for all possible combinations, the libraries were prescreened using syntactic filters to eliminate redundant sequences. These include all the sequences having: a) less than four different amino acids and more than two consecutive equal amino acids, b) more than three aliphatic amino acids (Ala, Val, Leu, and Ile) and/or more than two aromatic amino acids (Phe, Tyr, and Trp), c) less than one or more than three charged amino acids (Lys, Arg, His, Asp, and Glu), d) alternated hydrophobic and charged amino acids.

The a priori removal of sequences with low chemical diversity was designed to enhance identification of sequences that bind keratin by true affinity, hence eliminating sequences that permeate through non-specific interactions [Das, et al., J Chem Inf Model, 50(2):298-308 (2010); Vanhee, et al., Nucleic Acids Res, 38(Database issue):D545-D551 (2010)]. Further, by reducing the number of hydrophobic (either aliphatic or aromatic) amino acids, the probability of identifying SPPs with poor water solubility was reduced. Similarly, reducing the number of charged amino acids per sequence was a effort to lower risk of eliciting skin irritation, as is the case of poly-R. Finally, the fourth rule, i.e. the elimination of peptides including alternated hydrophobic and charged amino acids, rules out all sequences that may permeate the tissue via pore formation, such as the well-known cell-penetrating peptide [RW]ₙ [Vives, et al., Biochim Biophys Acta, 1786(2):126-138 (2008)]. Like highly charged peptides, pore forming peptides are regarded to be cytotoxic at concentrations required for significant enhancement of dermal drug delivery [Fattal, et al., Biochemistry, 33(21): 6721-6731 (1994)]. In fact, a goal of the screen was to identify sequences that, like SPACE™, permeate skin by migrating through the transcellular pathway. The application of these rules considerably reduced the library diversity, as in the heptapeptide case from ˜8.94 10⁹ down to ˜8.75 10⁴. The construction and pre-screening of the virtual libraries was performed using a code built in Java.

The finalized list was in turn utilized for constructing the coordinate files of each peptide using the open source graphic chemical structure visualization program PyMOL (The PyMOL Molecular Graphic System, Version 1.2r3pre, Schrodinger, LLC). The peptide structures were individually docked against human keratin using the docking software HADDOCK (version 2.1) [Dominguez, et al., J Am Chem Soc, 125(7):1731-1777 (2003)]. This program simulates protein-peptide interaction and through external software estimates the free energy of binding based on the evaluation of van der Waals interactions, hydrogen bonding, deformation penalty, hydrophobic effects, atomic contact energy, softened van der Waals interactions, partial electrostatic, additional estimation of the binding free energy, dipole-dipole interactions, and the presence of water [Dominguez, et al., J Am Chem Soc, 125(7):1731-1777 (2003); de Vries, et al., Proteins, 69(4):726-733 (2007)]. The coordinate file for the 2B regions from the central coiled-coil domains of human keratin5 and keratinl4, expressed in the keratinocytes of epidermis, was obtained from the RCSB Protein Data Bank (PDB, 3TNU) [Lee, et al., Nat Struct Mol Biol, 19(7):707-715 (2012)]. The same crystal structure had been previously employed for similar in silico modelling of five SPPs (SPACE™, poly-R, TD-1, DLP, and LP-12) [Kumar, et al., J Control Release, 199:168-178 (2015)].

To ensure binding specificity for each sequence, the variable region including the residues framed by the two cysteines was targeted to keratin, while the flanking regions Ala-Cys and Cys-Gly were not contacted. Default parameters (e.g. temperatures for heating/cooling steps, number of molecular dynamics sets per stage, etc.) were used in the docking procedure. The resulting docked structures were grouped in clusters and analyzed through built-in scoring functions that evaluate the free energy of binding in solution [Wang, et al., J Med Chem, 46(12): 2287-2303 (2003)]. The sequences were ranked accordingly to their average binding affinity for keratin. The best (lowest KD, highest affinity) keratin-binding sequences obtained from the first screening round and their KD values are reported in Table 2.

TABLE 2
Best keratin-binding sequences selected through in
silico screening of pentamer, hexamer, heptamer,
and octamer disulfide-bonded, cyclic peptides.

| Library | Sequence | SEQ ID NO: | K_{D} (M) |
|---|---|---|---|
| Pentamer (5) | ACSHNHTCG | 2 | 5.21 × 10⁻⁴ |
| Hexamer (6) | ACTHTGRNCG | 3 | 1.02 × 10⁻⁴ |
| Heptamer (7) | ACSATLQHSCG | 4 | 6.79 × 10⁻⁵ |
| Octamer (8) | ACNAHQARSTCG | 5 | 9.34 × 10⁻⁶ |

Short sequences offer lower binding affinity and thus potentially lower permeation enhancement. Alternatively, long sequences hold greater potential to possess high affinities but may consequently behave as ligands, thus preventing rather than promoting drug migration. Long sequences can also potentially exhibit lower diffusion due to increased size. For these reasons, pentamers were not tested in the remaining examples below, and the use of longer peptides (nonamers and beyond) were excluded. It is believed that hexamers, heptamers, and octamers hold the highest promise to afford the optimal combination of moderate affinity and diffusion and these candidates were the focus for validation of the in silico screening method. Interestingly, the SPACE™ sequence appeared in the top 0.02% of the heptamer list.

The top 5% of hexamers, heptamers, and octamers were used for a second round of screening, this time against the crystal structure of Cyclosporine A (PDB ID: 1CSA) to identify the best 100 sequences that bind both keratin and CsA. Notably, the best keratin-binding hexamer, heptamer, and octamer sequences were also found to possess high affinity for CsA. Further, both SPACE™ and Dermis Localizing peptide reported before (DLP) appeared among the final list of sequences. A sample list of the 10 top-binding heptamer sequences is reported in Table 3.

TABLE 3
Hepapeptide sequences selected for keratin and
CsA binding via sequential library screening
against 3TNU and 1CSA structures.

| ID | Sequence | SEQ ID NO: |
|---|---|---|
| 1 (SP7-1) | ACSATLQHSCG | 4 |
| 5 (SP7-2) | ACSLTVNWNCG | 6 |
| 10 (SP7-3) | ACLSVNHNACG | 7 |
| 17 (SPACE™) | ACTGSTQHQCG | 1 |
| 26 (SP7-5) | ACSASTNHNCG | 8 |
| 30 | ACSASQVHNCG | 9 |
| 40 | ACNGTGSHQCG | 10 |
| 50 | ACSVTTQHQCG | 11 |
| 75 | ACVSVTNHQCG | 12 |
| 100 (SP7-4) | ACTSTGRNACG | 13 |

Finally, the selected sequences were docked against keratin and CsA simultaneously to study the predicted binding mechanism. Many of the selected sequences showed the same binding mechanism displayed by SPACE™ and Dermis, wherein the peptide is interposed between keratin and CsA, thereby reinforcing the belief that peptides can serve as affinity mediators between the proteins of the skin and the permeating drug.

### Example 2

### SPP Bind to CsA and Keratin

### Materials and Methods

Peptide Synthesis and Other Reagents

The peptides were synthesized by Genscript Inc. (Piscataway, N.J., USA). Cyclosporine A (CsA) was purchased from Abcam (Cambridge, Mass., USA). 3H-CsA and 3H-Gly were purchased from Perkin Elmer (Waltham, Mass., USA). All other chemicals were obtained from Fisher Scientific (Fair Lawn, N.J., USA). Full thickness porcine skin was purchased from Lampire Biological Laboratories (Pipersville, Pa.) and stored at −80° C. Human adult epidermal keratinocytes (HEKa cells) and all cell culture materials were acquired from Life Technologies (Grand Island, N.Y.).

Mass Spectrometry and Affinity Chromatography

Five heptamer sequences selected from the in silico library screening and SPACE™ (positive control) all at the concentration of 25 mg/mL, CsA (5 mg/mL), and their binary SPPs/CsA mixtures were prepared in 45% (v/v) ethanol/water. Mass spectroscopic analysis was performed using Micromass

QTOF (Waters Corporation, Beverly, MA) with an electrospray ion source. Samples were diluted with Acetonitrile/Water containing 0.1% formic acid and then introduced via a Harvard Apparatus syringe pump at 10 μL/min flow rate. The capillary was held at 3.5 kV. Nitrogen was used as nebulizer, desolvation, and cone gas.

One hundred milligrams of dry Toyopearl AF-Epoxy-650 resin (epoxy density of 0.8 meq/g) was swollen in 20% v/v methanol for 2 h and then rinsed with 0.1 M carbonate buffer, pH 8.5. One milliliter of 50% v/v resin slurry in carbonate buffer was mixed with the peptide dissolved in DMF at a 30% molar ratio as compared to the resin functional density. The reaction was carried out overnight at room temperature under mild shaking. The supernatant was then collected and measured by UV spectroscopy at 220 nm to determine the peptide density on the solid phase. The resin was finally rinsed with 20% v/w ethanol and stored at 4° C. Using each resin, the adsorption isotherm of each skin-penetrating peptide was determined in a batch mode at room temperature. Nine aliquots of 10 mg of resin each were placed in microcentrifuge tubes, rinsed in 20% v/v methanol and equilibrated with PBS at pH 7.4. Solutions of human keratin (500 μL) with concentrations ranging from 0.05 to 1 mg/mL in PBS were added separately to the resin aliquots and incubated with gentle rotation for 2 hours. The samples were centrifuged, and the supernatants were collected and analyzed by UV absorbance at 280 nm to determine the protein concentration. The amount of bound keratin was calculated by mass balance. The data were fit to a Langmuir isotherm model where q, C, KD, and Qmax are the concentration of the bound protein (mg-protein/g-resin), the concentration of the free protein (mg-protein/mL-solution), the dissociation constant (mg/mL), and the maximum capacity (mg-protein/g-resin) respectively. The same process was repeated for Cyclosporine A, wherein the concentration of the peptide in the supernatant was measured by UV absorbance at 220 nm.

### Results

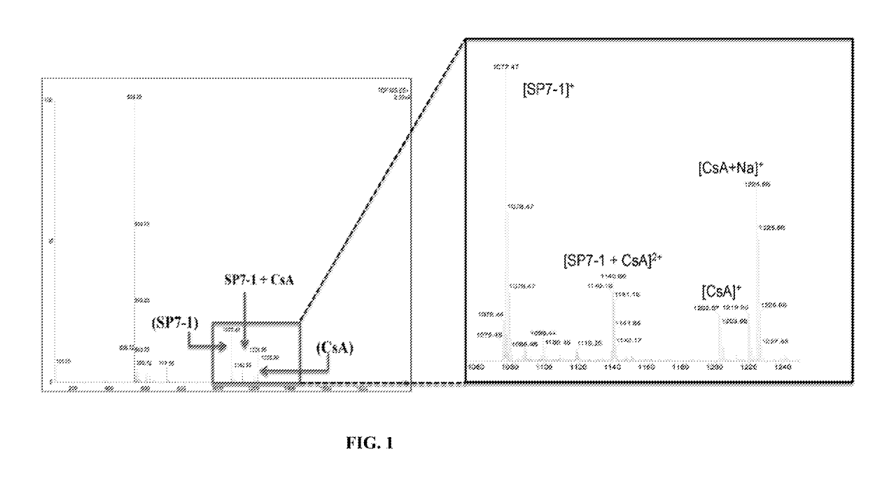

Out of all sequences identified in Example 1, seven were selected for experimental characterization, namely the best binding hexamer ACTHTGRNCG (SP6-1) (SEQ ID NO:3), a range of heptamers among the best binders ACSATLQHSCG (SP7-1) (SEQ ID NO:4), ACSLTVNWNCG (SP7-2) (SEQ ID NO:6), ACLSVNHNACG (SP7-3) (SEQ ID NO:7), ACTSTGRNACG (SP7-4) (SEQ ID NO:13), and ACSASTNHNCG (SP7-5) (SEQ ID NO:8), and the best binding octamer ACNAHQARSTCG (SP8-1) (SEQ ID NO:5). While not among the top ten candidates, SP7-5 was chosen due to its sequence homology with SPACE™. Binding of CsA to the selected SPPs was evaluated in solution by mass spectrometry. Notably, all selected sequences showed binding to CsA. FIG. 1 reports an exemplary spectrum showing the formation of a non-covalent binary complex between CsA and SP7-1. The peak of the SPP-CsA complex falls between those of SP7-1 and CsA.

The interaction between selected heptamer SPPs and keratin was experimentally estimated as well by determining the dissociation constant of each SPP-keratin complex via batch affinity chromatography. The affinity adsorbents were prepared by coupling each SPP to a chromatographic resin at a density of approx. 0.1 meq/g, which falls in the normal range for affinity chromatography. The resin was incubated with aqueous solution of keratin at increasing concentration within the range 0-1 mg/mL for a sufficient time to reach a binding equilibrium. The amounts of keratin captured by the resin were plotted as a function of the respective equilibrium protein concentration in solution and the data were fitted following a Langmuir isotherm equation. An exemplary binding isotherm (of SP7-1 for Keratin) is reported in FIG. 2 (also see FIG. 5A-5E). The experimental K_{D} values differ from the theoretical affinity value due to the on-resin avidity effect between multiple peptides and a single protein, i.e. due to the on-resin binding non-ideality. Interestingly, however, the measured K_{D} values show the same behavior as the predicted ones, as shown in Table 4, thereby confirming the validity of the in silico docking simulations. Taken together, these results validate the in silico selection of keratin-binding and CsA-binding sequences.

TABLE 4
Comparison of K_{D} values of SPP-Keratin interactions
experimentally measured vs. determined via in silico simulations.
Experimental Qₘₐₓ is also reported for all sequences.

| | | SEQ | | | |
|---|---|---|---|---|---|
| | | ID | | Experimental | Experimental |
| SPP ID | Sequence | NO: | In silico K_{D} | K_{D} | Qₘₐₓ |
| SP7-1 | ACSATLQHSCG | 4 | 6.79 10⁻⁵ M | 8.02 10⁻⁶ M | 4.16 mg/mL |
| | | | | | resin |
| SP7-2 | ACSLTVNWNCG | 6 | 7.42 10⁻⁵ M | 8.73 10⁻⁶ M | 4.09 mg/mL |
| | | | | | resin |
| SP7-3 | ACLSVNHNACG | 7 | 7.99 10⁻⁵ M | 9.4 10⁻⁶ M | 4.07 mg/mL |
| | | | | | resin |
| SPACE™ | ACTGSTQHQCG | 1 | 0.91 10⁻⁴ M | 1.06 10⁻⁵ M | 3.98 mg/mL |
| | | | | | resin |
| SP7-5 | ACSASTNHNCG | 8 | 0.84 10⁻⁴ M | 1.01 10⁻⁵ M | 4.01 mg/mL |
| | | | | | resin |
| SP7-4 | ACTSTGRNACG | 13 | 1.35 10⁻⁴ M | 1.58 10⁻⁵ M | 3.43 mg/mL |
| | | | | | resin |

### Example 3

### SSPs Penetrate Skin In Vitro

### Materials and Methods

Skin Penetration of Selected SPPs

Full thickness porcine skin was processed as reported in our earlier studies and integrity was verified by measuring the skin conductivity [Kumar, et al., J Control Release, 199:168-178 (2015)]. In vitro skin penetration studies were performed using Franz diffusion cells (FDCs) under the same conditions utilized in prior studies [Hsu, et al., Proc Natl Acad Sci USA, 108(38):15816-15821 (2011)]. The receptor compartment was filled with pH 7.4 phosphate buffered saline (PBS). The peptide-aided penetration of CsA was quantified following the same procedure. Test formulations used in this study were CsA alone (5 mg/mL) or with a SPP (25 mg/mL) dissolved in (45%, v/v) Ethanol/PBS solution. The pH of the PBS solution was adjusted to afford complete peptide dissolution. Penetration of CsA was measured alone (negative control), and in the presence of SPACE™ (positive control) and of selected SPPs. Test formulations were spiked with 3H-CsA (25 μCi/mL) for the purpose of quantitation. Test formulations were loaded and incubated as described. After 24 hours of incubation, the amount of CsA penetrated into different layers of skin and across the skin was quantified using a liquid scintillation counter (TRI-CARB 2100TR, Packard Instrument Company, Downers Grove, Ill.) as before.

Statistical Analysis

All the experiments were performed in triplicate, unless specified and the results are expressed as mean ±standard deviation (stdev). Student's t-test was used to compare two groups and one-way ANOVA followed by Bonferroni's correction for post-test comparisons was used when more than two groups were compared. The values of p<0.05, p<0.01, p<0.001 were considered significant with 95%, 99% and 99.9% confidence intervals, respectively. Statistical analyses were performed using GraphPad (Prism version 6) software.

### Results

The skin penetrating ability of the selected heptamer (SP7-1 through SP7-5) SPPs was evaluated in comparison with SPACE™. The skin permeation experiments were performed following the procedure adopted in previous studies [Kumar, et al., J Control Release, 199:168-178 (2015)]. Radiolabelled peptides were loaded on Franz diffusion cells (FDCs) and incubated at 37° C. with moderate stirring. After 24 hours, the various skin layers were harvested, dissolved and analyzed using a scintillation counter to determine the amount of peptide. Permeation results are reported in Table 5. Notably, all selected peptides were found able to penetrate skin, thereby confirming the validity of the screening approach for the de novo design of SPPs.

TABLE 5
Skin permeation of selected SPPs and SPACE ™ (μg/cm²).

| | SEQ ID | | | |
|---|---|---|---|---|
| SPP ID | NO: | SC + Epidermis | Dermis | Receptor |
| SP7-1 | 4 | 819.2 ± 62.2 | 149.7 ± 5.6 | 42.4 ± 8.5 |
| SP7-2 | 6 | 774.0 ± 110.2 | 166.7 ± 45.2 | 65.0 ± 14.1 |
| SP7-3 | 7 | 759.9 ± 73.4 | 39.5 ± 25.4 | 16.9 ± 11.3 |
| SP7-4 | 13 | 545.2 ± 79.1 | 53.7 ± 31.1 | 19.8 ± 19.8 |
| SP7-5 | 8 | 759.9 ± 62.1 | 124.3 ± 28.2 | 39.5 ± 8.5 |
| SPACE ™ | 1 | 782.5 ± 84.7 | 200.6 ± 59.3 | 96.0 ± 31.1 |

### Example 4

### CsA Skin Penetration is Enhanced with Selected SPPs

Following the same method, the ability of selected hexamer (ACTHTGRNCG (SEQ ID NO:3)), heptamer (SP7-1 through SP7-5 (SEQ ID NO:4, 6, 7, 13, 8), and octamer (ACNAHQARSTCG (SEQ ID NO:5)) SPPs to enhance the skin penetration of Cyclosporine A (CsA) was tested. In addition, the sequence ACGSGSGSGCG (SEQ ID NO:15) was added as a negative control, as the sequence [Gly-Ser]ₙ is usually employed for its biochemical inertia. Sequences SP7-2, SP7-4, and SP7-5 did not give any solubility problems in PBS pH 8.0, while pH values had to be adjusted to 8.5 for SP7-1 to achieve complete solubility. In presence of CsA, SP7-3 could not be fully solubilized and was hence not tested. The SPP-dependent enhancement of CsA delivery into skin was determined in comparison with SPACE™ (positive control) and 45% v/v ethanol (first negative control), and the non-binding heptamer ACGSGSGSGCG (SEQ ID NO:15) (second negative control).

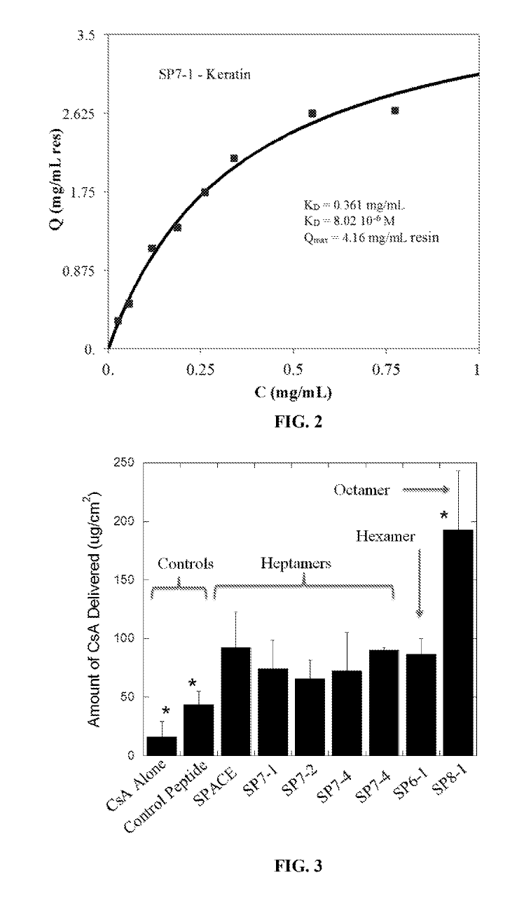

Results reported in FIG. 3 show that SP7-1 (ACSATLQHSCG (SEQ ID NO:4)) and SP7-5 (ACSASTNHNCG (SEQ ID NO:8)) sequences afforded a CsA permeation enhancement on par with SPACE™. SP-5 shows considerable sequence homology with SPACE™ (ACTGSTQHQCG (SEQ ID NO:1)). The low-binding heptamer afforded statistically significant less CsA penetration than SPACE™ (p>0.05), while all leading heptamers increased CsA penetration, although none of the heptamer sequences reported showed a statistically significant difference in skin penetration compared to SPACE™ peptide (p>0.05). Notably, the octamer afforded an outstanding penetration in the epidermis, likely due to its higher affinity for keratin (p<0.05 compared to SPACE™ peptide). Thus, taken together, these results show an appreciable correlation between computational ranking and CsA permeation enhancement, thereby validating the in silico selection method.

### Example 5

### Select SPP have Low Cytotoxicity

### Materials and Methods

Cell Culture and Cytotoxicity Assessment

HEKa cells were cultured in 1× keratinocyte Serum-Free Medium supplemented with 25 U/mL penicillin, 25 μg/mL streptomycin, and 50 μg/mL neomycin. Cultures were grown at 37° C. with 5% CO2. The cytotoxicity of SPPs was assessed using the MTT Cell Proliferation Assay (ATCC, Manassas, Va.). HEKa cells were seeded in 96-well microplates (Corning Inc., Corning, N.Y.) at a density of 5000 cells/well. Cultures were allowed to grow until they reached ˜80% confluency. Cells were then incubated with 200 μL of 10, 5, or 2.5 mg/mL of selected SPPs in media. The pH was adjusted to afford complete dissolution of peptides. Media only was used as a negative control, and media or SPP formulations without cells was used to subtract background. Cytotoxicity was assessed after overnight incubation. Viability was determined according to the manufacturer's recommended protocol using a SAFIRE, XFLUOR4, V4.50 microplate reader (Tecan Group Ltd, Morrisville, N.Y.).

### Results

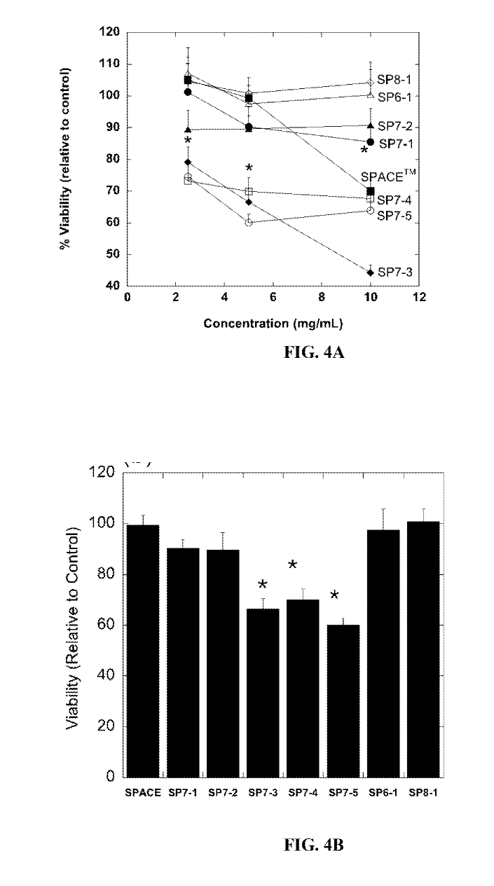

To assess the potential for skin toxicity, SPPs were incubated with HEKa cells overnight and % viability was determined. HEKa cells were used since keratinocytes are the primary cell-type in the skin, and therefore, represent a good estimate of the potential for skin irritation. Results are shown in FIG. 4A-4B.

Interestingly, the SPPs identified in silico showed wide variation in their toxicity profiles, notwithstanding the sequence similarity of the tested sequences. The hexamer and octamer SPPs appear to be non-toxic at concentrations as high as 10 mg/mL. Among heptamers, SP7-1 showed a similar toxicity trend with SPACE™ peptide, and was only toxic when at 10 mg/mL. SP-2 does appear to show some toxicity for all concentrations tested, however, the results were not statistically significant (p>0.05, for all concentrations) when compared to the control. On the other hand, SP7-3, SP7-4, and SP7-5 were significantly toxic (p <0.001) at only 1 mg/mL. Surprising is the toxicity of SP7-5 (ACSASTNHNCG (SEQ ID NO:8)), considering its homology with SPACE™ (ACSASTQHQCG (SEQ ID NO:1)). While models for predicting peptide toxicity are available [Gupta, et al., PLoS One, 8(9): e73957 (2013)], none seemed to apply to the sequences considered in this work. A comparison of toxicity of SPPs at a fixed concentration of 5 mg/ml can be seen in FIG. 4B.

Examples 1-5 show the development of an in silico screen for skin penetrating peptides that bind to keratin and CsA. Seven sequences identified in the screen were selected and validated by determining their ability to i) effectively bind keratin and CsA in solution, ii) individually penetrate skin and enhance CsA permeation through skin samples, and iii) avoid undesired effects on skin cells (keratinocytes) and proteins so as to ensure safety of the application. Notably, all the sequences demonstrated ability to penetrate skin and enhance transdermal penetration of CsA, some of which (SP7-1, SP7-5, and an SP8-1) equally to or better than SPACE™. Based on the ranking drawn by the post-screening analysis (and prior to empirical testing), some sequences (SP7-1, 2, and 3) were thought to afford higher CsA permeation as compared to SPACE™. Thus, while the ability of a peptide to act as a skin permeation enhancer is indeed related to its ability to act as a binding mediator between keratin and CsA, the in silico library screening against keratin pair 5/14 only does not completely describe the complexity of the affinity interactions that the peptide forms with the other skin proteins.

In order to select sequences with higher permeation enhancing power, the screening procedure should also include selection against other keratin isotopes and other skin proteins, provided that the necessary crystal structures were available. Further, there might also be other mechanisms underlying the observed permeation enhancement which are either not yet discovered and/or could not be translated into a computational screening step. One other aspect to consider is that, while SPP affinity for skin proteins and the target drug is necessary, the threshold of binding strength above which the SPP binds too tightly to skin proteins and hinders, rather than favoring, drug permeation is still unknown.

Also of interest is the wide variation in cytotoxicity among various SPPs identified here. This agrees with previous findings which showed large differences between SPACE™ peptide and previously known SPPs. Kumar, et al., J Control Release, 199:168-178 (2015). Further, previous study showed no observable relationship between cell toxicity and any single SPP molecular property, such as pI, hydrophilicity, and hydrophobicity. Therefore, it is understandable that each peptide induces a cytotoxic effect through its own unique combination of molecular properties. A number of peptides were identified with no observable toxicity (SP6-1 and SP8-1), as well peptides with a similar toxicity profile to SPACE™ peptide (S7P-2 and SP7-3). This result highlights the benefit of the method described here for identifying potentially optimized peptides in terms of efficacy and safety. However, a large difference in cytotoxicity was observed between SPACE™ peptide and SP-5 (99.4±3.9% viability and 60.1±2.6% viability, respectively, when HEKa cells were exposed to 5 mg/mL peptide in solution). The cause of the difference may be differing levels of off-target effects or rates of internalization.

While a broad range of peptides were identified as leading sequences, certain trends could be identified in terms of significance of specific motifs. For example, among the leading heptamers, the trimer QHQ appeared 7 times in the top 100 keratin binding sequences and the trimer NHN appeared 8 times. The trimer XHX, (X=Q or N) appeared 23 times and the trimer XHY and YHX (X=Q or N and Y is any amino acid) appeared 62 times. The trimer YHY (Y=any amino acid appeared 74 times).

While keratin binding appears important for skin penetration, optimum K_{d} also appears seems important for permeation. Low binding peptide ACGSGSGSGCG (SEQ ID NO:15) (K_{d}·0.1 M) exhibited no significant increase in CsA penetration. On the other hand, a high binding peptide (SP8-1, ACNAHQARSTCG (SEQ ID NO:5), K_{d}˜9.34×10⁻⁶) appears to exhibit rather superficial penetration of CsA. It is believed that this optimum dependence of penetration on K_{d} originates from the binding-diffusion process of peptides (and associated cargos) on skin keratin. Binding to CsA in the presence of CsA is also important to enable peptide partitioning into the skin; however, tight binding is likely to cause loss of its mobility.

The protocol described herein is computationally inexpensive, can be performed on standard commercially available hardware, and rapidly returns SPP sequences amenable for the transdermal delivery of a desired drug. Unlike other passive penetration enhancers known in the literature, including some peptides, which are not drug-specific and perform quite poorly for a number of pharmaceutically active ingredients, the sequences identified here were all capable of delivering CsA.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A polypeptide having a length in a range of from 5 to 100 amino acids, comprising the amino acid sequence X1-X2-X3-X4-X5-X6-X7-X8, wherein
X1 is T, S, L, N, or V;
X2 is G, A, L, S, V, or H;
X3 is S, T, V, N, or H;
X4 is T, L, V, N, Q, G, or H;
X5 is Q, N, H, V, S, R, T, or A;
X6 is absent or present, and if present: H, W, N, or R;
X7 is absent or present, can only be present if X6 is present, and if present: Q, S, N, or A; and
X8 is absent or present, can only be present if X6 and X7 are present, and if present: T,
wherein the polypeptide: (i) binds to a skin protein with a Kd of between 10⁻³ M and 10⁻⁸ M, (ii) is not a full-length naturally occurring protein, and (iii) does not include the amino acid sequence TGSTQHQ (SEQ ID NO: 301).

2. The polypeptide of claim 1, wherein the amino acid sequence X1-X2-X3-X4-X5-X6-X7-X8 is flanked by C residues.

3. The polypeptide of claim 1 or claim 2, comprising the amino acid sequence C—X1-X2-X3-X4-X5-X6-X7-X8-C.

4. The polypeptide of claim 3, comprising the amino acid sequence AC—X1-X2-X3-X4-X5-X6-X7-X8-CG.

5. The polypeptide of any one of claims 2-4, wherein the polypeptide is a cyclic peptide and a disulfide bond is present between said cysteine residues.

6. The polypeptide of any one of claims 1-5, wherein:
X1 is T, S, L, N, or V;
X2 is G, A, L, S, or V;
X3 is S, T, V, N, or H;
X4 is T, L, V, N, Q, G, or H;
X5 is Q, N, H, V, S, R, T, or A;
X6 is H, W, N, or R;
X7 is Q, S, N, or A; and
X8 is absent or present, and if present: T.

7. The polypeptide of claim 6, wherein X1-X2-X3-X4-X5-X6-X7-X8 is NAHQARST (SEQ ID NO: 305).

8. The polypeptide of claim 6, comprising the sequence ACNAHQARSTCG (SEQ ID NO: 5).

9. The polypeptide of any one of claims 1-5, wherein:
X1 is T, S, L, N, or V;
X2 is G, A, L, S, V, or H;
X3 is S, T, V, or N;
X4 is T, L, V, N, Q, G, or H;
X5 is Q, N, H, V, S, R, or T;
X6 is absent or present, and if present: H, W, N;
X7 is absent or present, can only be present if X6 is present, and if present: Q, S, N, or A; and
X8 is absent.

10. The polypeptide of claim 9, wherein X7 is absent and X1-X2-X3-X4-X5-X6 is THTGRN (SEQ ID NO: 303).

11. The polypeptide of claim 9, comprising the sequence ACTHTGRNCG (SEQ ID NO: 3).

12. The polypeptide of claim 9, wherein X6 and X7 are absent and X1-X2-X3-X4-X5 is SHNHT (SEQ ID NO: 302).

13. The polypeptide of claim 9, comprising the sequence ACSHNHTCG (SEQ ID NO: 2).

14. The polypeptide of any one of claims 1-5, wherein:
X1 is T, S, L, N, or V;
X2 is G, A, L, S, or V;
X3 is S, T, or V;
X4 is T, L, V, N, Q, or G;
X5 is Q, N, H, V, S, or R;
X6 is H, W, or N;
X7 is Q, S, N, or A; and
X8 is absent.

15. The polypeptide of claim 14, wherein X1-X2-X3-X4-X5-X6-X7 is selected from: SASQVHN (SEQ ID NO: 309), NGTGSHQ (SEQ ID NO: 310), SVTTQHQ (SEQ ID NO: 311), and VSVTNHQ (SEQ ID NO: 312).

16. The polypeptide of claim 14, comprising a sequence selected from:
(SEQ ID NO: 9)
ACSASQVHNCG,
(SEQ ID NO: 10)
ACNGTGSHQCG,
(SEQ ID NO: 11)
ACSVTTQHQCG,
and
(SEQ ID NO: 12)
ACVSVTNHQCG.

17. The polypeptide of any one of claims 1-5, wherein:
X1 is T, S, or L;
X2 is A, L, or S;
X3 is S, T, or V;
X4 is T, L, V, N, or G;
X5 is Q, N, H, or R;
X6 is H, W, or N;
X7 is S, N, or A; and
X8 is absent.

18. The polypeptide of claim 17, wherein X1-X2-X3-X4-X5-X6-X7 is selected from: SATLQHS (SEQ ID NO: 304), SLTVNWN (SEQ ID NO: 306), LSVNHNA (SEQ ID NO: 307), SASTNHN (SEQ ID NO: 308), and TSTGRNA (SEQ ID NO: 313).

19. The polypeptide of claim 17, comprising a sequence selected from:
(SEQ ID NO: 4)
ACSATLQHSCG,
(SEQ ID NO: 6)
ACSLTVNWNCG,
(SEQ ID NO: 7)
ACLSVNHNACG,
(SEQ ID NO: 8)
ACSASTNHNCG,
and
(SEQ ID NO: 13)
ACTSTGRNACG.

20. A polypeptide having a length in a range of from 5 to 100 amino acids, comprising the amino acid sequence set forth in any one of SEQ ID NOs: 326-417, 176-267, and 26-117, wherein the polypeptide: is not a full-length naturally occurring protein.

21. The polypeptide of claim 20, comprising the amino acid sequence set forth in any one of SEQ ID NOs: 176-267, and 26-117, wherein the polypeptide is a cyclic peptide and a disulfide bond is present between the two cysteine residues present in said sequence.

22. A polypeptide having a length in a range of from 5 to 100 amino acids, comprising the amino acid sequence C—X1-Xn-C (SEQ ID NO:16), or AC—X1-Xn-CG (SEQ ID NO:17), wherein each “X” is independently any amino acid excluding cysteine; wherein “n” is 2, 3, 4, 5, 6, 7, 8, 9, or 10; wherein a disulfide bond is present between the two cysteines; and wherein the polypeptide does not comprise the amino acid sequence set forth in any of SEQ ID NOs:1, 14, 23-25, 118-121, 301, 314, 323, and 418-421.

23. The polypeptide of claim 22, comprising one or more sequence motifs selected from the group consisting of NHN, QHN, NRN, and QRQ.

24. The polypeptide of any one of claims 1-23, wherein the skin protein is selected from the group consisting of keratin, collagen, plectin, actin, and tubulin.

25. The polypeptide of claim 24, wherein the skin protein is keratin.

26. The polypeptide of claim 25, wherein the keratin is keratin 5, keratin 14, or a combination thereof.

27. The polypeptide of any one of claims 1-26, having a length in a range of from 7 to 30 amino acids.

28. The polypeptide of claim 27, having a length in a range of from 7 to 12 amino acids.

29. The polypeptide of any one of claims 1-28, wherein the polypeptide is bound or conjugated to a therapeutically active agent.

30. The polypeptide of claim 29, wherein the therapeutically active agent is a polypeptide, nucleic acid, or small molecule.

31. The polypeptide of claim 29, wherein the therapeutically active agent is a dermatological agent.

32. The polypeptide of claim 29, wherein the therapeutically active agent is Cyclosporine A.

33. A pharmaceutical composition comprising the polypeptide of any one of claims 1-32.

34. The pharmaceutical composition of claim 33, further comprising a therapeutically active agent.

35. The pharmaceutical composition of claim 33 or claim 34, wherein the polypeptide is bound or conjugated to the therapeutically active agent.

36. The pharmaceutical composition of any one of claims 33-35, wherein the therapeutically active agent is a polypeptide, nucleic acid, or small molecule.

37. The pharmaceutical composition of any one of claims 33-35, wherein the therapeutically active agent is a dermatological agent.

38. The pharmaceutical composition of any one of claims 33-35, wherein the therapeutically active agent is Cyclosporine A.

39. A method of treating a subject in need thereof comprising administering to the subject the polypeptide of any one of claims 1-32 in combination with a therapeutically active agent.

40. The method of claim 39, wherein the polypeptide and the therapeutically active agent are together in the same pharmaceutical composition.

41. The method of claim 40, wherein the polypeptide is bound or conjugated to the therapeutically active agent.

42. The method of claim 39, wherein the polypeptide and the therapeutically active agent are in separate pharmaceutical compositions.

43. The method of any one of claims 39-42, wherein the polypeptide and therapeutically active agent are administered topically to the subject.

44. The method of claim 43, wherein the polypeptide and therapeutically active agent are administered topically to the skin of the subject.

45. The method of any one of claims 39-44, wherein the therapeutically active agent is a polypeptide, nucleic acid, or small molecule.

46. The method of any one of claims 39-44, wherein the therapeutically active agent is a dermatological agent.

47. The method of any one of claims 39-44, wherein the therapeutically active agent is cyclosporine A.

48. The method of any one of claims 39-47, wherein the subject has a dermatological condition, disease, or disorder.
